(19) United States
(12) Patent Application Publication
(10) Pub. No.: US 2026/0258413 A1
(43) Pub. Date: Sep. 3, 2026

(54) MiRNA FOR CONTROLLING EXPRESSION OF IN VIVO DELIVERY CONSTRUCTS

(71) Applicant: CREATE Medicines, Inc., Cambridge, MA (US)

(72) Inventors: Daniel GETTS, Medfield, MA (US); Yuxiao Wang, Belmont, MA (US); Ewa Grudzien Nogalska, Lexington, MA (US)

(21) Appl. No.: 19/322,061

(22) Filed: Sep. 8, 2025

Related U.S. Application Data
(63) Continuation of application No. PCT/US2024/019227, filed on Mar. 8, 2024.
(60) Provisional application No. 63/489,307, filed on Mar. 9, 2023.
(60) Provisional application No. 63/582,220, filed on Sep. 12, 2023.
(60) Provisional application No. 63/593,159, filed on Oct. 25, 2023.

(51) Int. Cl. C12N 15/113; A61K 9/1272; A61K 9/51; A61K 31/7105; A61K 40/31; A61K 40/42; C07K 14/705; C07K 16/30; C07K 16/32; C12N 15/67; C12N 15/88

(52) U.S. Cl. CPC C12N 15/113; A61K 9/1272; A61K 9/5123; A61K 31/7105; A61K 40/31; A61K 40/4205; C07K 14/70535; C07K 16/30; C07K 16/303; C07K 16/32; C12N 15/67; C12N 15/88; A61K 2239/13; A61K 2239/21; A61K 2239/22; C12N 2310/141; C12N 2320/31

(57) ABSTRACT

Compositions and methods for making and using engineered phagocytic cells that express a chimeric antigen receptor having an enhanced phagocytic activity for stable and durable expression are described and can be suitably used for immunotherapy in cancer or infection.

### CROSS REFERENCE

This application is a continuation of the PCT application No. PCT/US24/19227 filed on Mar. 8, 2024, which claims the benefit of U.S. Provisional Application No. 63/489,307, filed on Mar. 9, 2023, U.S. Provisional Application No. 63/582,220, filed on Sep. 12, 2023, and U.S. Provisional Application No. 63/593,159 filed on Oct. 25, 2023, each of which is incorporated herein by reference in its entirety.

### SEQUENCE LISTING

This application contains a Sequence Listing which is submitted electronically in XML format and is hereby incorporated by reference in its entirety. Said XML copy, created on Apr. 19, 2024, is named 56371-746_601_SL.xml and is 100,680 bytes in size.

### BACKGROUND

Myeloid cells are a major cellular compartment of the immune system comprising monocytes, dendritic cells, tissue macrophages, and granulocytes. Although myeloid cells have many functions, including phagocytosis and their ability to activate T cells, harnessing these functions for therapeutic uses has remained elusive. Newer avenues are therefore sought for using other cell types towards development of improved therapeutics, including but not limited to T cell malignancies.

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery.

### SUMMARY

The present disclosure is related to immunotherapy using myeloid cells, including phagocytic cells of the immune system, particularly macrophages. It is an object of the invention disclosed herein to harness one or more of these functions of myeloid cells for therapeutic uses. For example, it is an object of the invention disclosed herein to express a recombinant polypeptide in myeloid cells when delivered systemically in vivo. In one aspect, with help of the recombinant technology an engineered nucleic acid, (e.g., mRNA) encoding the recombinant polypeptide is administered in vivo, the engineered nucleic acid comprises sequences incorporated in the 5′- and/or 3′ untranslated regions that allows inhibition of expression of the recombinant polypeptide in non-myeloid cells. For example, it is an object of the invention disclosed herein to minimize off-target effects in myeloid cell engineering in vivo. For example, it is an object of the invention disclosed herein to harness the phagocytic activity of myeloid cells, including engineered myeloid cells, for therapeutic uses. For example, it is an object of the invention disclosed herein to harness the ability of myeloid cells, including engineered myeloid cells, to promote T cell activation. For example, it is an object of the invention disclosed herein to harness the ability of myeloid cells, including engineered myeloid cells, to promote secretion of tumoricidal molecules. For example, it is an object of the invention disclosed herein to harness the ability of myeloid cells, including engineered myeloid cells, to promote recruitment and trafficking of immune cells and molecules. For example, it is an object of the invention disclosed herein to generate activated myeloid cells in vivo. The present disclosure provides innovative methods and compositions that can successfully render a myeloid cell to express an exogenous nucleic acid in vivo, e.g., an exogenous recombinant nucleic acid comprising a sequence encoding a polypeptide that has a therapeutic effect. For example, provided herein are innovative methods and compositions whereby, recombinant nucleic acids administered to an organism may express preferentially or specifically in myeloid cells in vivo.

The present disclosure involves making and using engineered myeloid cells (e.g., CD14+ cells, such as macrophages or other phagocytic cells, which can attack and kill (ATAK) diseased cells directly and/or indirectly, such as cancer cells and infected cells. Engineered myeloid cells, such as monocytes and macrophages and other phagocytic cells, can be prepared by incorporating nucleic acid sequences (e.g., mRNA, plasmids, viral constructs) encoding a gene of interest, such as a chimeric fusion protein (CFP), that has an extracellular binding domain specific to disease associated antigens (e.g., cancer antigens), into the cells using, for example, nucleic acid technology, synthetic nucleic acids, gene editing techniques (e.g., CRISPR), transduction (e.g., using viral constructs), electroporation, or nucleofection. It has been found that myeloid cells can be engineered to have a broad and diverse range of activities. For example, it has been found that myeloid cells can be engineered to express a chimeric fusion protein (CFP) containing an antigen binding domain to have a broad and diverse range of activities. For example, it has been found that myeloid cells can be engineered to have enhanced phagocytic activity such that upon binding of the CFP to an antigen on a target cell, the cell exhibits increased phagocytosis of the target cell. It has also been found that myeloid cells can be engineered to promote T cell activation such that upon binding of the CFP to an antigen on a target cell, the cell promotes activation of T cells, such as T cells in the tumor microenvironment. The engineered myeloid cells can be engineered to promote secretion of tumoricidal molecules such that upon binding of the CFP to an antigen on a target cell, the cell promotes secretion of tumoricidal molecules from nearby cells. The engineered myeloid cells can be engineered to promote recruitment and trafficking of immune cells and molecules such that upon binding of the CFP to an antigen on a target cell, the cell promotes recruitment and trafficking of immune cells and molecules to the target cell or a tumor microenvironment.

The present disclosure is based on the important finding that myeloid cells can be engineered to express a gene of interest and can be targeted to a site of inflammation, infection or tumor. Myeloid cells thus engineered can overcome at least some of the limitations of CAR-T cells, including being readily recruited to solid tumors; having an engineerable duration of survival, therefore lowering the risk of prolonged persistence resulting in aplasia and immunodeficiency; myeloid cells cannot be contaminated with T cells; myeloid cells can avoid fratricide, for example because they do not express the same antigens as malignant T cells; and myeloid cells have a plethora of anti-tumor functions that can be deployed. In some respects, engineered myeloid derived cells can be safer immunotherapy tools to target and destroy diseased cells. However, engineering myeloid cells for enhancing gene expression for non-viral DNA therapy remains a significant challenge.

Moreover, myeloid cells, such as macrophages, have been ubiquitously found in the tumor environment (TME) and are notably the most abundant cells in some tumor types. As part of their role in the immune system, myeloid cells, such as macrophages, are naturally engaged in clearing diseased cells. The present invention relates to harnessing myeloid cell function specifically for targeting, killing and directly and/or indirectly clearing diseased cells as well as the delivery payloads such as antigens and cytokines.

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery.

The present disclosure provides innovative methods and compositions that can successfully electroporate, transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity. Provided herein is a composition comprising a nucleic acid comprising (i) DNA sequence encoding an mRNA or (ii) the mRNA sequence, wherein the mRNA sequence comprises (i) a 5′ UTR sequence and (ii) a 3′ UTR sequence, wherein the 5′ UTR is at least 45 nucleotides in length and a sequence encoding a target gene or protein therebetween. In some embodiments, the 5′ UTR sequence, and/or the 3′ UTR sequence can comprise a non-native sequence, that is, a sequence that is not present in an unmodified transcript. In some embodiments, the nucleic acid sequence is recombinant nucleic acid sequence. In some embodiments, the nucleic acid” or nucleic acid sequence is engineered. In some embodiments, the nucleic acid or nucleic acid sequence is synthetic. In some embodiments, the nucleic acid or nucleic acid sequence is in vitro transcribed. In some embodiments, the nucleic acid or nucleic acid sequence is isolated or purified.

In some embodiments, the nucleic acid, e.g., an engineered nucleic acid, an in vitro transcribed (IVT) mRNA, a synthetic or modified nucleic acid as described herein is not conjugated to or associated with a lipid nanoparticle (LNP). In some embodiments, the engineered nucleic acid is delivered systemically in vivo to alter myeloid cells in vivo, the engineered nucleic acid being encapsulated in a nanoparticle. In some embodiments, the nanoparticle comprises non-lipid polymeric compounds.

In some embodiments, the engineered nucleic acid is formulated in a lipid nanoparticle for systemic delivery to alter myeloid cells in vivo.

In some embodiments, the nucleic acid, e.g., an engineered nucleic acid, an in vitro transcribed mRNA, a synthetic or modified nucleic acid as described herein is electroporated into a cell. In some embodiments, the nucleic acid, e.g. an IVT mRNA comprises a 3′UTR and a 5′UTR. In some embodiments, the 3′ UTR sequence is followed by a poly A sequence. In some embodiments, the poly A sequence is at least 100 nucleotides long. In some embodiments, the poly A sequence is at least about 50, 60, 70, 80, 90, 100, 110, 120, 130, 140, 150, 160, 170, 180, 190 or 200 nucleotides long. In some embodiments, the poly A sequence is greater than 200 nucleotides long. In some embodiments, within the 5′ UTR, a translation start site is at least 15 nucleotides downstream of the 5′ end of the mRNA. In some embodiments, a translation start site is at least 20 nucleotides downstream of the 5′ end of the mRNA. In some embodiments, the translation start site is at least 25 nucleotides downstream of the ribosome binding site. In some embodiments, the translation start site is at least 30 nucleotides downstream of the ribosome binding site. In some embodiments, the 5′ end of the nucleic acid comprises a methyl guanylate cap. In some embodiments, the nucleic acid comprises a single translational start site. In some embodiments, the mRNA coding sequence is 100-10,000 nucleotides long. In some embodiments, the nucleic acid sequence comprises a 5′ UTR sequence selected from sequences described herein. In some embodiments, the nucleic acid sequence comprises a 3′ UTR sequence selected from the sequences described herein. Provided herein is a composition comprising a cell comprising the composition described in the above section, wherein in some embodiments the cell is a myeloid cell, a CD14+ cell, a CD16− cell, or a CD14+/CD16− cell or the cell is a T cell. Also provided herein is a pharmaceutical composition comprising the composition described above; and a pharmaceutical acceptable excipient.

In one aspect, provided herein is a method of expressing an exogenous polypeptide from a recombinant nucleic acid in a myeloid cell, the method comprising contacting a heterogenous population of cells comprising the myeloid cell and a non-myeloid cell with a composition comprising the recombinant polynucleic acid, wherein the recombinant nucleic acid comprises (a) a sequence encoding the exogenous polypeptide, and (b) one or more miRNA target sequences; wherein the exogenous polypeptide is expressed in the myeloid cell of the heterogenous population of cells, and wherein the exogenous polypeptide is not substantially expressed in the non-myeloid of the heterogenous population of cells. “Contacting” comprises contacting the heterogenous population on the external surface of a cell in the heterogenous population of cells.

In one aspect, provided herein is a method of expressing an exogenous polypeptide from a recombinant nucleic acid in a myeloid cell, the method comprising contacting a heterogenous population of cells comprising the myeloid cell and a non-myeloid cell with a composition comprising the recombinant polynucleic acid, wherein the recombinant nucleic acid comprises (a) a sequence encoding the exogenous polypeptide, (b) a sequence comprising (i) one or more miRNA target sequences, and (ii) a sequence encoding a polypeptide required for expression of the exogenous polypeptide; wherein the exogenous polypeptide is expressed in the myeloid cell of the heterogenous population of cells, and wherein the exogenous polypeptide is not substantially expressed in the non-myeloid of the heterogenous population of cells.

In one aspect, provided herein is a method of expressing an exogenous polypeptide from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the exogenous polypeptide, and (b) one or more miRNA target sequences; and wherein the recombinant nucleic acid lacks a sequence encoding a viral protein or portion thereof, and wherein the exogenous polypeptide is expressed in the cell that is the first cell type of the heterogenous population of cells, and wherein the exogenous polypeptide is not substantially expressed in the cell that is the second cell type of the heterogenous population of cells.

In one aspect, provided herein is a method of expressing an exogenous polypeptide from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the exogenous polypeptide, (b) a sequence comprising (i) one or more miRNA target sequences, and (ii) a sequence encoding a polypeptide required for expression of the exogenous polypeptide; wherein the composition lacks a sequence encoding a viral protein or portion thereof, and wherein the exogenous polypeptide is expressed in the cell that is the first cell type of the heterogenous population of cells, and wherein the exogenous polypeptide is not substantially expressed in the cell that is the second cell type of the heterogenous population of cells.

In one aspect, provided herein is a method of expressing a chimeric fusion protein from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the chimeric fusion protein, and (b) one or more miRNA target sequences; and wherein the chimeric fusion protein is expressed in the cell that is the first cell type of the heterogenous population of cells, and wherein the chimeric fusion protein is not substantially expressed in the cell that is the second cell type of the heterogenous population of cells.

In one aspect, provided herein is a method of expressing a chimeric fusion protein from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the exogenous polypeptide, (b) a sequence comprising (i) one or more miRNA target sequences, and (ii) a sequence encoding a polypeptide required for expression of the exogenous polypeptide; wherein the chimeric fusion protein is expressed in the cell that is the first cell type of the heterogenous population of cells, and wherein the chimeric fusion protein is not substantially expressed in the cell that is the second cell type of the heterogenous population of cells.

In some embodiments, the non-myeloid cell of the heterogenous population of cells expresses a microRNA (miRNA) that comprises a sequence that is reverse-complementary to at least one of the one or more miRNA target sequences, or a fragment thereof.

In some embodiments, the non-myeloid cell of the heterogenous population of cells comprises any one of a hepatocyte, a lymphoid cell (e.g., T cell, B cell), an alveolar cell, a cardiac cell, a kidney cell, a stromal cell, an epithelial cell, a muscle cell, an ocular cell, a nerve cell, a brain cell and a skeletal cell. A myeloid cell may be for example, a macrophage, a monocyte, a dendritic cell, a monocyte derived cell, or a CD14+ cell.

In some embodiments, the myeloid cell of the heterogenous population of cells does not substantially express a miRNA that is reverse-complementary to at least one of the one or more miRNA target sequences.

In some embodiments, the recombinant nucleic acid is an mRNA.

In some embodiments, the exogenous polypeptide is a recombinant polypeptide that activates a myeloid cell when expressed in the myeloid cell ex vivo.

In some embodiments, activating the myeloid cell comprises activating or increasing chemotaxis of the myeloid cell compared to a cell not expressing the recombinant polypeptide.

In some embodiments, activating the myeloid cell comprises activating phagocytosis of the myeloid cell.

In some embodiments, the exogenous polypeptide comprises a recombinant receptor protein, a recombinant cytokine, chemokine, growth factor or a portion thereof.

In some embodiments, the composition comprising the recombinant nucleic acid further comprises a nucleic acid delivery vehicle.

In some embodiments, the nucleic acid delivery vehicle comprises a lipid molecule.

In some embodiments, the nucleic acid delivery vehicle comprises a cationic lipid. In some embodiments, the nucleic acid delivery vehicle comprises a lipid nanoparticle. In some embodiments, the nucleic acid delivery vehicle is a polymer.

In one aspect, provided herein is a composition comprising one or more recombinant polynucleic acids engineered for expressing a sequence encoding an exogenous polypeptide in a myeloid cell, the recombinant nucleic acid comprising, the sequence encoding the exogenous polypeptide, and one or more miRNA target sequences, wherein the one or more miRNA target sequences comprises a sequence that is reverse-complementary to at least one miRNA sequence or fragment thereof, that is expressed in a non-myeloid cell, and is absent in a myeloid cell.

In some embodiments, the one or more recombinant polynucleic acids comprises (a) a sequence encoding the exogenous polypeptide, (b) a sequence comprising (i) one or more miRNA target sequences, and (ii) a sequence encoding a polypeptide required for expression of the exogenous polypeptide.

In some embodiments, the one or more recombinant polynucleic acids comprises a vector comprising the sequence encoding the human polypeptide, and the one or more miRNA target sequences.

In some embodiments, the one or more recombinant polynucleic acids is an mRNA molecule.

In some embodiments, the mRNA comprises a 5′-UTR and a 3′-UTR.

In some embodiments, the one or more miRNA target sequences are incorporated in the 5′-UTR and the 3′-UTR.

In some embodiments, each of the one or more miRNA target sequence comprises a target sequence for an miRNA expressed in a non-myeloid cell and wherein expression of the miRNA is absent in a myeloid cell.

In some embodiments, the miRNA target sequence comprises a target sequence for an miRNA that is expressed in a hepatocyte, a lymphoid cell (e.g., T cell, B cell), an alveolar cell, a cardiac cell, a kidney cell, a stromal cell, an epithelial cell, a muscle cell, an ocular cell, a nerve cell, a brain cell or a skeletal cell.

In some embodiments, the miRNA target sequence comprises a target sequence for an miRNA that is expressed in an epithelial cell and a hepatocyte. In some embodiments, the miRNA target sequence comprises the sequence set forth in SEQ ID NO: 4. In some embodiments, the miRNA target sequence comprises the sequence set forth in SEQ ID NO: 5.

In some embodiments, the miRNA target sequence comprises the sequence set forth in SEQ ID NO: 6. In some embodiments, the miRNA target sequence comprises the sequence set forth in SEQ ID NO: 7. In some embodiments, the miRNA target sequence comprises the sequence set forth in SEQ ID NO: 8. In some embodiments, the miRNA target sequence comprises the sequence set forth in SEQ ID NO: 9. In some embodiments, the mRNA 3′ UTR comprises one or more sequences selected from SEQ ID NOs: 5, 7 and 9. In some embodiments, the mRNA 5′ UTR comprises one or more sequences selected from SEQ ID NOs: 4, 6 and 8.

In some embodiments, the composition further comprises a nucleic acid delivery vehicle.

In some embodiments, the nucleic acid delivery vehicle comprises a lipid molecule.

In some embodiments, the nucleic acid delivery vehicle comprises a cationic lipid.

In some embodiments, the nucleic acid delivery vehicle comprises a lipid nanoparticle.

In some embodiments, the nucleic acid delivery vehicle is a polymer.

In some embodiments, the exogenous polypeptide is a therapeutic polypeptide.

In some embodiments, the exogenous polypeptide comprises a receptor protein a cytokine, chemokine, growth factor or a portion thereof.

In some embodiments, the exogenous polypeptide comprises a chimeric antigen receptor.

In one aspect, provided herein is a method of expressing an exogenous polypeptide in a myeloid cell of a subject, the method comprising administering to the subject the composition of any one of the above embodiments; wherein after administering, the exogenous polypeptide expresses in the myeloid cell of the subject and does not substantially express in a non-myeloid cell of the subject.

In one aspect, provided herein is a method of making a composition for expressing an exogenous polypeptide selectively in a myeloid cell, the method comprising: (i) synthesizing a polynucleotide sequence that is a reverse complement of an miRNA that is expressed in a non-myeloid cell, and wherein expression of the miRNA is absent in a non-myeloid cell; (ii) synthesizing an engineered polynucleic acid comprising a sequence encoding the exogenous polypeptide and an miRNA target sequence comprising the polynucleotide sequence that is the reverse complement of the miRNA in (i), wherein the sequence encoding the exogenous polypeptide and the miRNA target sequence are operably linked.

Provided herein is a method of treating a subject with a disease or condition comprising administering the pharmaceutical composition described herein to a subject in need thereof. Also provided herein is a method of expressing a protein encoded by a nucleic acid in a cell, the method comprising (a) incorporating into the cell ex vivo a nucleic acid comprising (i) DNA encoding an mRNA or (ii) the mRNA, and (b) expressing a protein encoded by a sequence of the mRNA; wherein the mRNA comprises (i) a 5′ UTR sequence; a sequence encoding a protein or polypeptide and (ii) a 3′ UTR sequence, wherein expression of the protein or polypeptide is detectable up to at least 24 hours, at least 48 hours, or at least 72 hours after (a).

Provided herein is a method of expressing a protein encoded by a nucleic acid in a cell, the method comprising (a) incorporating into the cell ex vivo a nucleic acid comprising (i) DNA encoding an mRNA or (ii) the mRNA, and (b) expressing a protein encoded by a sequence of the mRNA; wherein the mRNA comprises (i) a 5′ UTR sequence and (ii) a 3′ UTR sequence, wherein the 5′ UTR is at least 45 nucleotides in length.

In some embodiments, the 3′ UTR is followed by a poly A tail and the poly A tail is enzymatically added to the 3′end of the mRNA. In some embodiments, the 3′ UTR is followed by a poly A tail and the poly A tail is an encoded poly A tail.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 shows data indicating α-globin 3′UTR with four miR122-5p binding sites suppresses mRNA expression in hepatocytes (Huh-7 cells) but not in primary monocytes.

FIG. 2 shows data indicating α-globin 3′UTR with four miR192-5p binding sites suppresses mRNA expression in hepatocytes but not in primary monocytes.

FIG. 3 depicts data indicating α-globin 3′UTR with four miR192-3p binding sites. no significant change was observed with and without the miRNA.

FIG. 4 shows data indicating α-globin 3′UTR with four miR27b-5p binding sites prolongs mRNA expression specifically in primary monocytes.

FIG. 5 shows data indicating α-globin 3′UTR with four miR27b-3p binding sites suppresses mRNA expression in both hepatocytes and primary monocytes (no cells specificity).

FIG. 6 shows data indicating α-globin 3′UTR with four miR10a-5p binding sites. No significant effect with and without the miRNA was observed.

FIG. 7 shows α-globin 3′UTR with four miR10a-3p binding sites. No significant effect with and without the miRNA was observed.

FIG. 8 shows data indicating GC33-CD89 lambda 10 and lambda 4 demonstrate prolong expression in primary monocytes.

FIG. 9A shows AI based mRNA sequence improvement and folding to offer increased stability to mRNA.

FIG. 9B (left) shows data showing an exemplary screening of several codon optimized mRNA structures, and sequences for preparing stable long-lasting mRNA constructs with different UTRs; several combinations for the constructs were generated using AI algorithms.

FIG. 9B (right) shows microRNA sites stabilize mRNA in myeloid cells, which is confirmed across various cell types. Expression of chimeric receptors continued beyond 7 days after introduction of the mRNA (e.g. transfection), when miRNA were administered.

FIG. 10 shows data indicating results obtained of unprecedented high longevity of primary monocytes by improving the coding sequence of the mRNA along with incorporation of the microRNA sites as shown in the figures.

FIGS. 11A and 11B demonstrate experimental set up and constructs used for understanding whether the UTRs with the miRNA sequences influence the half-life of a generic mRNA. For this luciferase construct (F. Luc) was used as a surrogate marker for mRNA comprising the designed UTRs. Myeloid cells were cultured ex vivo as indicated. Cells are from human donor.

FIG. 12 demonstrates mode of differentiation of the myeloid cell types into iDCs and macrophages from isolated immature cells in culture while expressing the luciferase encoded by the mRNA with specialized UTRs.

FIG. 13 shows expression of the markers for distinct cell types of myeloid cells expressing the F. Luc mRNA.

FIG. 14 shows data indicating prolonged expression of the f. Luc expressing mRNA, endowed by the modified UTR.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Myeloid cells are recently shown to provide a strong platform for therapeutic intervention in various diseases including cancer, because these cells can migrate chemotactically to a locus of infection, inflammation, and tumor, can phagocytose and kill bacteria and parasites, can scavenge dead or apoptotic cells and can attack tumor cells or cells harboring mutation or an aberrant phenotype. A myeloid cell is usually short lived inside the body. A myeloid cell can be engineered ex vivo to augment one or more of the functions related to phagocytosis or chemotaxis and introduced into a subject having a disease, for the engineered myeloid cell to target and attack the diseased cells in the subject, such as tumor cells or infected cells. However, myeloid cells are complex and can rapidly undergo change of state in which its effectiveness as a phagocytic cell may be diminished. In this respect, engineering or manipulating a myeloid cell can pose various challenges. For example, introducing plasmid DNA in a myeloid cell can lead to transformation of a monocyte cell into a M2 phenotype, where the cells may lose chemotactic movement, and surface expression of chemoattracted receptors. A monocyte or macrophage with altered phenotype following introduction of DNA comprising a gene of interest may become less effective in its innate functions such as phagocytosis and chemotaxis.

Provided herein are methods and compositions of engineering a myeloid cell. In some embodiments, the engineering is ex vivo. In some embodiments, an exogenous polynucleotide sequence may be introduced in vivo that is designed to be specifically taken up and expressed by a myeloid cell in vivo. In some embodiments, the design of the exogenous polynucleotide sequence, e.g., a recombinant polynucleotide sequence is such that it allows expression of the polypeptide encoded by the exogenous polynucleic acid in myeloid cell and prevents the expression of the polypeptide encoded by the exogenous polynucleic acid in a non-myeloid cell. Of importance, compositions and methods are provided herein to express a polynucleotide sequence in a myeloid cell. The method and compositions described herein allow preferential and specific expression of a polypeptide encoded by the recombinant polynucleic acid.

The myeloid cell expressing the exogenous polynucleotide sequence should retain its chemotactic, phagocytic and cytotoxic function in order to perform a therapeutically effective function in a living body.

Provided herein are polynucleotide compositions for improved expression of an encoded protein or polypeptide in a myeloid cell. In some embodiments, a polynucleic acid is sought to be expressed preferentially in a myeloid cell residing in a heterogenous population of cells comprising myeloid cells and non-myeloid cells. In some embodiments a myeloid cell is a monocyte, a monocyte derived macrophage, a macrophage, a CD14+ cell, a CD14+ and CD16+ cell, a CD14+ and CD16− cell. In some embodiments, the non-myeloid cell type comprises an alveolar cell, a cardiac cell, a kidney cell, a stromal cell, an epithelial cell, a T cell, a B cell, an NK cell, a muscle cell, an ocular cell, a nerve cell, a brain cell, a skeletal cell or a hepatocyte.

In some embodiments, the polynucleotide is messenger RNA.

The instant disclosure is directed to a non-viral method and compositions for expressing a exogenous genetic material, such as a polynucleotide in a myeloid cell to ensure robust and prolonged expression in a myeloid cell, and to ensure that the myeloid cell expressing the exogenous genetic material is fully functional and capable of actively migrating to the cite of infection or inflammation or tumor, that it responds to chemotactic signals in vivo for it to be localized, for example to a tumor, and that it is actively phagocytic.

Prolonged expression of a polynucleic acid in a myeloid cell has been found to be highly challenging. First there are technical difficulty associated with transfecting monocytes and macrophages. As professional phagocytes, monocytes and macrophages are endowed with many potent degradative enzymes that can disrupt nucleic acid integrity and make gene transfer into these cells an inefficient process. Monocytes and macrophages undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery. For multiple reasons as is known to one of skill in the art, alternatives to viral gene delivery is always sought for safety issues. However, most of the original transfection techniques enjoyed only limited success with macrophages. This is due to the requirement of fairly high gene copy numbers required to efficiently transfect cells and the relatively high degree of toxicity associated with the process whereby the host cell membrane is made permeable to DNA. The various transfection methods used to introduce foreign DNA into mammalian cells include: DEAE-dextran, calcium phosphate coprecipitation, cationic lipid vehicles, and physical disruption of the host cell membranes by electroporation or nucleofection. All these approaches have been used with varying degrees of success on macrophages.

Provided herein are methods and compositions for producing engineered myeloid cells (including, but not limited to, neutrophils, monocytes, myeloid dendritic cells (mDCs), mast cells and macrophages), designed to specifically express a gene of interest (GOI), such as a protective gene in a disease. A gene of interest may be a gene or a polynucleic acid bearing a sequence encoding corrected wild-type protein sequence that can substitute a mutated non-functional sequence in a subject that does not express a functional protein. A gene of interest may be an immunoprotective gene, a chemokine, a cytokine, a secreted protein, a cytoplasmic protein, a membrane protein or a nuclear protein. In some embodiments, a gene of interest is a chimeric fusion protein (CFP), alternatively termed a chimeric antigenic receptor (CAR).

In one embodiment, a myeloid cell can be engineered to express a CAR (chimeric antigen receptor), also termed chimeric fusion protein (CFP) that can bind a target protein or biomolecule or a target cell. These engineered myeloid cells can therefore attack and kill target cells directly (e.g., by phagocytosis) and/or indirectly (e.g., by activating T cells). In some embodiments, the target cell is a cancer cell.

While a polynucleotide encoding a CFP (or CAR) may be used throughout the specification as an exemplary embodiment, the composition and methods disclosed herein are applicable for expressing a polynucleotide encoding any gene of interest, e.g., a synthetic polynucleotide.

While cancer is one exemplary embodiment described in detail in the instant disclosure, the methods and technologies described herein are contemplated to be useful in targeting an infected or otherwise diseased cell inside the body. In some embodiments, the methods and compositions may be utilized in introducing any polynucleotide sequence encoding a protein to enhance macrophage function, such as a transcription enhancer, or a cytokine or a chemokine. The method can be used to target the myeloid cell to a pathogen and engulf the pathogen and at the same time present suitable antigens to T cells for inducing the lymphocyte cascade and protective immunity. In some embodiments, an exemplary pathogen may be Bacillus anthracis, Clostridium botulinum, Fracisella tularensis, Variola major, Salmonella sp., Mycobacterium tuberculosis, Listeria monocytogenes, Campylobacter jejuni, Yersinia enterocolitica, or protozoans, for example, Cryptosporidium parvum, Cyclospora cayatanensis, Giardia lamblia, Entamoeba histolytica, Toxoplasma gondi, Naegleria fowleri, Balamuthia mandrillaris, or fungi.

In some embodiments, the methods and compositions disclosed herein may be useful in generating engineered myeloid cells for expressing a gene to compensate for a deficient gene or replace a mutated gene product with a correct wild-type protein, such as in monogenic disorders.

Provided herein are compositions and methods for treating diseases or conditions, such as cancer. The compositions and methods provided herein utilize human myeloid cells, including, but not limited to, neutrophils, monocytes, myeloid dendritic cells (mDCs), mast cells and macrophages, to target diseased cells, such as cancer cells. The compositions and methods provided herein can be used to eliminate diseased cells, such as cancer cells and or diseased tissue, by a variety of mechanisms, including T cell activation and recruitment, effector immune cell activation (e.g., CD8 T cell and NK cell activation), antigen cross presentation, enhanced inflammatory responses, reduction of regulatory T cells and phagocytosis. For example, the myeloid cells can be used to sustain immunological responses against cancer cells.

In one aspect, provided herein is a method of expressing a polynucleic acid in a myeloid cell, such as a CD14+ human myeloid cell. In some embodiments, provided herein is an engineered polynucleic acid. In some embodiments, the engineered polynucleic acid is suitable for efficient expression on a polypeptide encoded by the engineered polynucleic acid in a myeloid cell. In some embodiments, the polynucleic acid is RNA. In some embodiments, the RNA is mRNA. In some embodiments, the mRNA encodes a protein that is expressed in the myeloid cell. In some embodiments, the mRNA encoded protein is a cytoplasmic protein. In some embodiments, the mRNA encoded protein is a nuclear protein. In some embodiments, the mRNA encoded protein is a membrane protein. In some embodiments, the mRNA encoded protein is a secreted protein.

In some embodiments an mRNA comprising a sequence encoding a gene of interest is expressed in a myeloid cell, wherein the mRNA is modified for efficient expression in a myeloid cell. In some embodiments, the mRNA is modified at the termini for enhanced and/or prolonged expression in a myeloid cell. In some embodiments, the termini include a 5′terminus, and/or a 3′terminus modification.

In some embodiments, the modification may be in the mRNA chemistry, that is in the nucleotides of the mRNA. In some embodiments, the modification is introduced into the mRNA at the time of mRNA formation. In some embodiments, the modification is introduced into the mRNA after the mRNA is formed.

In one embodiment, the poly A tail of the mRNA is designed for improving the efficiency of expressing a polypeptide encoded by a sequence within the mRNA in a myeloid cell.

In some embodiments, the 5′-UTR is specifically designed or chosen for improving the efficiency of expressing a polypeptide encoded by a sequence within the mRNA in a myeloid cell.

In some embodiments, the 3′-UTR is specifically designed or chosen for improving the efficiency of expressing a polypeptide encoded by a sequence within the mRNA in a myeloid cell.

Also provided herein is a pharmaceutical composition comprising a composition described herein, such as an engineered nucleic acid described herein, a vector described herein, a polypeptide described herein or a cell described herein; and a pharmaceutically acceptable excipient. The term “nucleic acid” may be used to designate a synthesized, recombinant, engineered or in vitro transcribed nucleic acid. In some embodiments, a nucleic acid is not naturally occurring.

Provided herein is a composition comprising a nucleic acid comprising: (i) DNA encoding an mRNA or (ii) the mRNA, wherein the mRNA comprises (i) a 5′ UTR sequence and (ii) a 3′ UTR sequence, wherein the 5′ UTR is at least 45 nucleotides in length and a sequence encoding a protein or polypeptide, wherein the protein or polypeptide encoded by the nucleic acid when expressed in the myeloid cell is detected in the cell for at least up to 72 hours. Provided herein is a composition comprising a nucleic acid comprising: an mRNA wherein the mRNA comprises (i) a 5′ UTR sequence and (ii) a 3′ UTR sequence, where (i) and (ii) flanks (iii) a sequence encoding a protein or polypeptide; wherein the 5′ UTR sequence is selected from the sequences provided herein; and the 3′ UTR sequence selected from the sequences provided herein; wherein the protein or polypeptide encoded by the nucleic acid when expressed in the myeloid cell is detected in the cell for at least up to 72 hours. Provided herein is a composition comprising a nucleic acid comprising: (i) DNA encoding an mRNA or (ii) the mRNA, wherein the mRNA comprises (i) a 5′ UTR sequence and (ii) a 3′ UTR sequence, wherein the 5′ UTR is at least 45 nucleotides in length and a sequence encoding a protein or polypeptide, and wherein the mRNA comprises an enzymatically added poly A tail; wherein the protein or polypeptide encoded by the nucleic acid when expressed in the myeloid cell is detected in the cell for at least up to 72 hours. In some embodiments, the composition is not conjugated to or associated with a lipid nanoparticle (LNP). In some embodiments, the nucleic acid comprises one or more modified nucleotide bases, wherein a fraction of the total number of uridine bases are modified to a pseudouridine, a 1-methyl-pseudouridine or a 5-methoxyuridine. In some embodiments, less than 50% of the total number of uridine bases are modified to a pseudouridine, a 1-methyl-pseudouridine or a 5-methoxyuridine. In some embodiments, the protein or polypeptide encoded by the nucleic acid when expressed in the myeloid cell is detected in the cell for at least up to 72 hours. In some embodiments the 5′ UTR is at least 20 nucleotides in length. In some embodiments the 5′ UTR is at least 30 nucleotides in length. In some embodiments the 5′ UTR is at least 60 nucleotides in length. In some embodiments the 5′ UTR is at least 100 nucleotides in length.

In one aspect, methods and compositions are disclosed herein for in vivo expression, the method comprises expression of a recombinant polypeptide encoded by a recombinant nucleic acid preferentially or specifically in myeloid cells while avoiding off-target expression in one or more non-myeloid cell types.

In one embodiment, provided herein are compositions and methods for manipulating the 3′UTR of the recombinant nucleic acid such that the recombinant nucleic acid is degraded in certain non-myeloid cells, or is not translated to generate the recombinant polypeptide in the certain non-myeloid cells. In some embodiments, the certain non-myeloid cells are either abundantly present in the vicinity of the myeloid cells or may be specific cell types in which the recombinant polypeptide is particularly undesired or harmful. Alternatively, in some embodiments, provided herein are compositions and methods for manipulating the 5′UTR of the recombinant nucleic acid such that the recombinant nucleic acid is degraded in certain non-myeloid cells, or is not translated to generate the recombinant polypeptide in the certain non-myeloid cells. In some embodiment, the non-myeloid cell where expression of the recombinant polypeptide is undesired is an epithelial cell, or a cell of epithelial lineage.

In one embodiment, the nucleic acid is an mRNA comprising a poly A sequence that is enzymatically added. In one embodiment, the nucleic acid is an mRNA comprising a poly A sequence that is enzymatically added. In some embodiments, the nucleic acid is an mRNA comprising a poly A sequence that is encoded by the plasmid which comprises the template for generating the mRNA by in vitro transcription (IVT). The template for IVT is a linearized plasmid. Typically, the length of the poly A is controlled when encoded by the plasmid and is less controlled when enzymatically added. An mRNA product comprising an enzymatically added poly A tail may be tailored at best to contain a narrowed range of the number of A-residues. The in vitro transcribed mRNA is thereafter purified prior.

In some embodiments, the nucleic acid is encapsulated in a lipid nanoparticle. In some embodiments, the lipid nanoparticle comprises a cationic lipid, and at least one of: a non-cationic lipid, a neutral lipid, cholesterol and a conjugated lipid. In some embodiments, the LNP is less than 250 nm is diameter. In some embodiments, the LNP is less than 250 nm is diameter. In some embodiments, the LNP is less than 150 nm is diameter. In some embodiments, the LNP is about 100 nm is diameter. In some embodiments, the nucleic acid is encapsulated in a liposome.

In some embodiments, the nucleic acid comprises a poly A sequence downstream of the 3′ UTR sequence. In some embodiments, the poly A sequence is at least 50 nucleotides long. In some embodiments, the poly A sequence is at least 60, 70, 80, or 90 nucleotides long. In some embodiments, the poly A sequence is at least 100 nucleotides long. In some embodiments, the poly A sequence is at least 110 nucleotides long. In some embodiments, the poly A sequence is at least 120 nucleotides long. In some embodiments, the poly A sequence is at least 130 nucleotides long. In some embodiments, the poly A sequence is at least 140 nucleotides long. In some embodiments, the poly A sequence is at least 150, 160, 170, 180, 190 or 200 nucleotides long. In some embodiments, the within the 5′ UTR, a translation start site is at least 15 nucleotides downstream of the 5′ end. In some embodiments, the translation start site is at least 20 nucleotides downstream of the 5′ end. In some embodiments, the translation start site is at least 25 nucleotides downstream of the 5′ end. In some embodiments, the translation start site is at least 30 nucleotides downstream of the 5′ end. In some embodiments, the nucleic acid comprises a single translational start site.

In some embodiments, the nucleic acid comprises a 5′ methyl guanylate cap. In some embodiments, the nucleic acid comprises a Cap 0 structure. In some embodiments, the Cap 0 structure is introduced into the mRNA cotranscriptionally using anti-reverse capping analog (ARCA).

In some embodiments, the nucleic acid is 20-20,000 nucleotides long. In some embodiments, the nucleic acid is 20-22 nucleotides long. In some embodiments, the nucleic acid is 20-50 nucleotides long. In some embodiments, the nucleic acid is 20-100 nucleotides long. In some embodiments the nucleic acid is a small interfering RNA. In some embodiments, the nucleic acid is an inhibitory RNA. In some embodiments, the nucleic acid is a tRNA. In some embodiments, the nucleic acid is single stranded. In some embodiments, the nucleic acid is double stranded.

In some embodiments, the nucleic acid is an mRNA. In some embodiments, the mRNA is 50-20,000 nucleotides long.

In some embodiments, the nucleic acid is an engineered nucleic acid. In some embodiments, the nucleic acid is recombinant nucleic acid. In some embodiments, the nucleic acid is in vitro transcribed mRNA. In some embodiments, the nucleic acid is a synthesized nucleic acid.

In some embodiments, the nucleic acid is isolated.

In some embodiments, the nucleic acid is purified.

In some embodiments, the nucleic acid comprises at least 1 modified nucleotide.

In some embodiments, the nucleic acid comprises at least 10% modified nucleotides.

In some embodiments, the nucleic acid comprises at least 20% modified nucleotides.

In some embodiments, the nucleic acid comprises at least 30%, 40%, or 50% modified nucleotides. In some embodiments, less than 70% of the uridine residues in the nucleic acid are modified.

In some embodiments, less than 50% of the uridine residues in the nucleic acid are modified.

In some embodiments, the modified nucleotide is a pseudouridine, 1-methyl-pseudouridine or a 5-methoxyuridine that replaces a uridine.

In some embodiments, the nucleic acid comprises about 10%, about 12%, about 14%, about 16%, about 18%, about 20%, about 21%, about 22%, about 23%, about 24%, about 25%, about 26% about 27%, about 28%, about 29%, about 30%, about 31%, about 32%, about 33%, about 34%, about 35%, about 36%, about 37%, about 38%, about 39% about 40% or more of the uridine residues of the nucleic acid that are modified to a pseudouridine, 1-methyl-pseudouridine or a 5-methoxyuridine.

Provided herein is a composition comprising a cell comprising the composition of any one of embodiments described above.

In some embodiments, the cell is a myeloid cell, a CD14+ cell, a CD16− cell, or a CD14+/CD16− cell.

In some embodiments, the cell is a T cell.

In some embodiments, the expression of the protein in the cell is detectable for at least 72 hours.

Provided herein is a pharmaceutical composition comprising the composition of any one of embodiments described above; and a pharmaceutical acceptable excipient.

Provided herein is a method of treating a subject with a disease or condition comprising administering the pharmaceutical composition described above to a subject in need thereof.

Provided herein is a method of expressing a protein encoded by a nucleic acid in a CD14+ cell, the method comprising: (a) incorporating into the CD14+ cell a nucleic acid comprising (i) DNA encoding an mRNA or (ii) the mRNA wherein the mRNA comprises a sequence encoding the protein, and (b) expressing the protein encoded by a sequence of the mRNA; wherein the mRNA comprises (i) a 5′ UTR sequence and (ii) a 3′ UTR sequence, wherein expression of the protein is detectable in the CD14+ cell up to at least 72 hours after (a).

In some embodiments, expression of the protein is detectable according to an immunoassay up to at least 72 hours after (a).

In some embodiments, expression of the protein is detectable in at least 20% of the cells after at least 72 hours after (a).

Provided herein is a method of expressing a protein encoded by a nucleic acid in a CD14+ cell, the method comprising incorporating into the CD14+ cell a nucleic acid comprising (i) DNA encoding an mRNA or (ii) the mRNA, and expressing a protein encoded by a sequence of the mRNA; wherein the mRNA comprises (i) a 5′ UTR sequence and (ii) a 3′ UTR sequence, flanking a sequence encoding the protein; wherein the 5′ UTR is at least 45 nucleotides in length.

In some embodiments, the nucleic acid comprises a 5′ UTR sequence selected from the sequences provided herein. In some embodiments, the nucleic acid sequence comprises a 3′ UTR sequence selected from the sequences provided herein.

In some embodiments, the nucleic acid comprises a poly A tail, wherein the poly A tail is enzymatically added to the 3′end of the mRNA or is an encoded poly A tail.

In some embodiments, the nucleic acid sequence comprises a 5′ UTR sequence and that of the 3′-UTR can be tested and paired in a construct in order to maximize the expression level and stability of the mRNA over time. Provided herein is a method for expressing a nucleic acid in a myeloid cell such that the nucleic acid is detectable in the cell at least up to 72 hours after introducing the nucleic acid in the myeloid cell. Provided herein is a method for expressing an mRNA encoding the protein in a myeloid cell such that the protein is detectable in the cell at least up to 72 hours after introducing the mRNA in the myeloid cell. In some embodiments, the 5′ UTR is at least 50, at least 60, at least 70, at least 80 or at least 100 nucleotides in length. In some embodiments, the nucleic acid comprises a poly A tail of 50-200 adenylate residues (SEQ ID NO: 27), and wherein the poly A tail is enzymatically added to the mRNA.

In some embodiments, the mRNA is targeted for myeloid cell-specific expression. In some embodiments, the mRNA encodes a sequence that is specifically expressed in a myeloid cell.

In some embodiments, the mRNA is introduced in the myeloid cell as a naked nucleic acid.

In some embodiments, the mRNA is introduced in the myeloid cell via a delivery vehicle, e.g., an LNP. In some embodiments, the lipid nanoparticle comprises a cationic lipid, and at least one of: a non-cationic lipid, a neutral lipid, cholesterol and a conjugated lipid.

In some embodiments, incorporating comprises transfecting. In some embodiments, incorporating comprises electroporating.

In some embodiments, incorporating comprises culturing the cell in the presence of the nucleic acid, wherein the nucleic acid is present at a concentration of at most about 500 micrograms/mL.

In some embodiments, incorporating comprises culturing the cell in the presence of the nucleic acid, wherein the nucleic acid is present at a concentration of at most about 250 micrograms/mL. In some embodiments, incorporating comprises culturing the cell in the presence of the nucleic acid, wherein the nucleic acid is present at a concentration of at most about 100 micrograms/mL. In some embodiments, incorporating comprises culturing the cell in the presence of the nucleic acid, wherein the nucleic acid is present at a concentration of at most about 50 micrograms/mL.

In some embodiments, incorporating comprises culturing the cell in the presence of the nucleic acid, wherein the nucleic acid is present at a concentration of from about 1 microgram/mL to about 500 micrograms/mL.

In some embodiments, incorporating comprises culturing the cell in the presence of the nucleic acid, wherein the nucleic acid is present at a concentration of from about 10 microgram/mL to about 100 micrograms/mL.

In some embodiments, incorporating comprises culturing the cell in the presence of the nucleic acid, wherein the nucleic acid is present at a concentration of from about 10 microgram/mL to about 50 micrograms/mL. In some embodiments, incorporating comprises incorporating in vivo. In some embodiments, the nucleic acid comprises a sequence encoding a membrane protein. In some embodiments, the nucleic acid comprises a sequence encoding a cytosolic protein. In some embodiments, the nucleic acid comprises a sequence encoding a recombinant protein. In some embodiments, the nucleic acid is an mRNA, wherein the mRNA is 50-20,000 nucleotides long.

In some embodiments, the nucleic acid comprises at least 1 modified nucleotide. In some embodiments, the nucleic acid comprises at least 10% modified nucleotides. In some embodiments, the nucleic acid comprises at least 20% modified nucleotides. In some embodiments, the nucleic acid comprises at least 30%, 40%, or 50% modified nucleotides. In some embodiments, less than 70% of the uridine residues in the nucleic acid are modified. In some embodiments, less than 50% of the uridine residues in the nucleic acid are modified. In some embodiments, the modified nucleotide is a pseudouridine, 1-methyl-pseudouridine or a 5-methoxyuridine that replaces a uridine.

In some embodiments, the nucleic acid is encapsulated in a lipid nanoparticle. In some embodiments, the lipid nanoparticle comprises a cationic lipid, and at least one of: a non-cationic lipid, a neutral lipid, cholesterol and a conjugated lipid. In some embodiments, the lipid nanoparticle comprises a cationic lipid, a non-cationic lipid and a conjugated lipid. In some embodiments, the conjugated lipid is a PEGylated lipid. In some embodiments, the PEG moiety is PEG-2000.

In some embodiments, following incorporation of the nucleic acid in a population of CD14+ cells ex vivo, at least 80% of cells in the population of CD14+ cells express the protein encoded by the nucleic acid at 24 hours. In some embodiments, following incorporation of the nucleic acid in a population of CD14+ cells ex vivo, at least 70% of the population express the protein encoded by the nucleic acid after 48 hours. In some embodiments, the protein is detectable in CD14+ cells up to 72 hours following incorporation of the nucleic acid. In some embodiments, the protein is detectable in CD14+ cells up to 96 hours following incorporation of the nucleic acid. In some embodiments, the protein is detectable in CD14+ cells up to 5 days following incorporation of the nucleic acid.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, may encompass variations of +/−30% or less, +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

An “antigen presenting cell” or “APC” as used herein includes professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

A “biological sample” can refer to any tissue, cell, fluid, or other material derived from an organism.

The term “epitope” can refer to any protein determinant, such as a sequence or structure or amino acid residues, capable of binding to an antibody or binding fragment thereof, a T cell receptor, and/or an antibody-like molecule. Epitopic determinants typically consist of chemically active surface groups of molecules such as amino acids or sugar side chains and generally have specific three-dimensional structural characteristics as well as specific charge characteristics. A “T cell epitope” can refer to peptide or peptide-MHC complex recognized by a T cell receptor.

An engineered cell, such as an engineered myeloid cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric fusion protein (CFP).

The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages. Immune responses include adaptive immune responses. The adaptive immune system can react to foreign molecular structures, such as antigens of an intruding organism. Unlike the innate immune system, the adaptive immune system is highly specific to apathogen. Adaptive immunity can also provide long-lasting protection. Adaptive immune reactions include humoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis.

In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

A “ligand” can refer to a molecule which is capable of binding or forming a complex with another molecule, such as a receptor. A ligand can include, but is not limited to, a protein, a glycoprotein, a carbohydrate, a lipoprotein, a hormone, a fatty acid, a phospholipid, or any component that binds to a receptor. In some embodiments, a receptor has a specific ligand. In some embodiments, a receptor may have promiscuous binding to a ligand, in which case it can bind to several ligands that share at least a similarity in structural configuration, charge distribution or any other physicochemical characteristic. A ligand may be a biomolecule. A ligand may be an abiotic material. For example, a ligand may be a negative charged particle that is a ligand for scavenger receptor MARCO. For example, a ligand may be TiO₂, which is a ligand for the scavenger receptor SRA1.

The term “major histocompatibility complex (MHC)”, “MHC molecule”, or “MHC protein” may refer to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins are loaded with antigens that usually originate from endogenous proteins or from pathogens present inside cells and are then presented to naïve or cytotoxic T-lymphocytes (CTLs). HLA class II proteins are present on antigen presenting cells (APCs), including but not limited to dendritic cells, B cells, and macrophages. They mainly present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α- and β-chains that interact to form a peptide-binding groove that is more open than class I peptide-binding grooves.

HLA alleles are typically expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRα (DRA1), and one or more genes HLA-DRβ (DRB1 and DRB3, -4 or -5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB* 11:01, HLA-DRB*15:01, and HLA-DRB*07:01.

A “myeloid cell” can refer broadly to cells of the myeloid lineage of the hematopoietic cell system, and can exclude, for example, the lymphocytic lineage. Myeloid cells comprise, for example, cells of the granulocyte lineage and monocyte lineages. Myeloid cells are differentiated from common progenitors derived from the hematopoietic stem cells in the bone marrow. Commitment to myeloid cell lineages may be governed by activation of distinct transcription factors, and accordingly myeloid cells may be characterized as cells having a level of plasticity, which may be described as the ability to further differentiate into terminal cell types based on extracellular and intracellular stimuli. Myeloid cells can be rapidly recruited into local tissues via various chemokine receptors on their surface. Myeloid cells are responsive to various cytokines and chemokines.

A myeloid cell, for example, may be a cell that originates in the bone marrow from a hematopoietic stem cell under the influence of one or more cytokines and chemokines, such as G-CSF, GM-CSF, Flt3L, CCL2, VEGF and S100A8/9. In some embodiments, the myeloid cell is a precursor cell. In some embodiments, the myeloid cell may be a cell having characteristics of a common myeloid progenitor, or a granulocyte progenitor, a myeloblast cell, or a monocyte-dendritic cell progenitor or a combination thereof. A myeloid can include a granulocyte or a monocyte or a precursor cell thereof. A myeloid can include an immature granulocyte, an immature monocyte, an immature macrophage, an immature neutrophil, and an immature dendritic cell. A myeloid can include a monocyte or a pre-monocytic cell or a monocyte precursor. In some cases, a myeloid cell as used herein may refer to a monocyte having an M0 phenotype, an M1 phenotype or an M2 phenotype. A myeloid can include a dendritic cell (DC), a mature DC, a monocyte derived DC, a plasmacytoid DC, a pre-dendritic cell, or a precursor of a DC. A myeloid can include a neutrophil, which may be a mature neutrophil, a neutrophil precursor, or a polymorphonucleocyte (PMN). A myeloid can include a macrophage, a monocyte-derived macrophage, a tissue macrophage, a macrophage of an M0, an M1 or an M2 phenotype. A myeloid can include a tumor infiltrating monocyte (TIM). A myeloid can include a tumor associated monocyte (TAM). A myeloid can include a myeloid derived suppressor cell (MDSC). A myeloid can include a tissue resident macrophage. A myeloid can include a tumor associated DC (TADC). Accordingly, a myeloid cell may express one or more cell surface markers, for example, CD11b, CD14, CD15, CD16, CD38, CCR5, CXCR1, CXCR2, CXCR4, CD66, Lox-1, CD11c, CD64, CD68, CD163, CCR2, CCR4, CCR7, CX3CR1, HLA-DR, CD1c, CD83, CD141, CD209, MHC-II, CD123, CD303, CD304, a SIGLEC family protein and a CLEC family protein. In some cases, a myeloid cell may be characterized by a high or a low expression of one or more of cell surface markers, for example, CD11b, CD14, CD15, CD16, CD66, Lox-1, CD11c, CD64, CD68, CD163, CCR2, CCR5, HLA-DR, CD1c, CD83, CD141, CD209, MHC-JJ, CD123, CD303, CD304 or a combination thereof.

“Phagocytosis” is used interchangeably with “engulfment” and can refer to a process by which a cell engulfs a particle, such as a cancer cell or an infected cell. This process can give rise to an internal compartment (phagosome) containing the particle. This process can be used to ingest and or remove a particle, such as a cancer cell or an infected cell from the body. A phagocytic receptor may be involved in the process of phagocytosis. The process of phagocytosis can be closely coupled with an immune response and antigen presentation. The processing of exogenous antigens follows their uptake into professional antigen presenting cells by some type of endocytic event. Phagocytosis can also facilitate antigen presentation. For example, antigens from phagocytosed cells or pathogens, including cancer antigens, can be processed and presented on the cell surface of APCs.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties.

A “receptor” can refer to a chemical structure composed of a polypeptide, which transduces a signal, such as a polypeptide that transduces an extracellular signal to a cell. A receptor can serve to transmit information in a cell, a cell formation or an organism. A receptor comprises at least one receptor unit and can contain two or more receptor units, where each receptor unit comprises a protein molecule, e.g., a glycoprotein molecule. A receptor can contain a structure that binds to a ligand and can form a complex with the ligand. Signaling information can be transmitted by a conformational change of the receptor following binding with the ligand on the surface of a cell.

The term “antibody” refers to a class of proteins that are generally known as immunoglobulins, including, but not limited to IgG1, IgG2, IgG3, and IgG4, IgA (including IgAQ1 and IgA2), IgD, IgE, IgM, and IgY. The term “antibody” includes, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a V_{L} and/or a V_{H} domain. Antibodies can be from any animal origin. Antigen-binding antibody fragments, including single-chain antibodies, can comprise variable region(s) alone or in combination with one or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

A nucleic acid as described herein can contain a nucleotide sequence that is not naturally occurring. An engineered nucleic acid may be synthesized in the laboratory. A nucleic acid may be prepared by using recombinant DNA technology, for example, enzymatic modification of DNA, such as enzymatic restriction digestion, ligation, and DNA cloning. A nucleic acid can be DNA, RNA, analogues thereof, or a combination thereof. A recombinant DNA may be transcribed ex vivo or in vitro, such as to generate a messenger RNA (mRNA). An mRNA as described herein may be isolated, purified and used to electroporate or transfect a cell. An mRNA as described herein can be an in vitro transcribed mRNA. A nucleic acid, such has an mRNA, described herein can be synthetic. A nucleic acid, such has an mRNA, described herein can be encoded by a vector. A nucleic acid, such has an mRNA, described herein may encode a protein or a polypeptide.

The process of introducing or incorporating a nucleic acid into a cell can be via electroporation, transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ refers to natural infections of humans or animals with wild-type viruses.

The term “vector” can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The terms “spacer” or “linker” as used in reference to a fusion protein/polypeptide refers to a peptide sequence that joins two other peptide sequences of the fusion protein/polypeptide. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein/peptide region may include Gly, Asn and Ser, or any permutation of amino acid sequences containing Gly, Asn and Ser. Other near neutral amino acids, such as Thr and Ala, also can be used in the linker sequence.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which can refer to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a cell or a population of cells to a subject in need thereof. In some embodiments, treating comprises administering to the subject one or more of engineered cells described herein, e.g. one or more engineered myeloid cells, such as phagocytic cells. Treating comprises treating a disease or a condition or a syndrome, which may be a pathological disease, condition or syndrome, or a latent disease, condition or syndrome. In some cases, treating, as used herein may comprise administering a therapeutic vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

A “fragment” can refer to a portion of a protein or nucleic acid. In some embodiments, a fragment retains at least 50%, 75%, or 80%, or 90%, 95%, or even 99% of the biological activity of a reference protein or nucleic acid.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

The terms “neoplasia” or “cancer” refers to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

The term “vaccine” is to be understood as meaning a composition for generating immunity for the prophylaxis and/or treatment of diseases (e.g., neoplasia/tumor/infectious agents/autoimmune diseases). Accordingly, vaccines as used herein are medicaments which comprise engineered nucleic acids, or cells comprising and expressing a nucleic acid and are intended to be used in humans or animals for generating specific defense and protective substance by vaccination. A “vaccine composition” can include a pharmaceutically acceptable excipient, carrier or diluent. Aspects of the present disclosure relate to use of the technology in preparing a phagocytic cell-based vaccine.

The term “pharmaceutically acceptable” refers to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

MicroRNAs (miRNAs) may be described as a class of small non-coding RNAs that play important roles in regulating gene expression. Most miRNAs are transcribed from DNA sequences into primary miRNAs and processed into precursor miRNAs, and finally mature miRNAs. miRNAs are an average 22 nucleotides in length. Most miRNAs are transcribed from DNA sequences into primary miRNAs (pri-miRNAs) and processed into precursor miRNAs (pre-miRNAs) and mature miRNAs. In most cases, miRNAs interact with the 3′ UTR of target mRNAs to suppress expression.

In most cases, miRNAs interact with the 3′ untranslated region (3′ UTR) of target mRNAs to induce mRNA degradation and translational repression. Although target gene mRNA levels might not be altered by miRNA over-expression, reduced protein levels are expected. However, interaction of miRNAs with other regions, including the 5′ UTR, coding sequence, and gene promoters, have also been reported. Under certain conditions, miRNAs can also activate translation or regulate transcription. The interaction of miRNAs with their target genes is dynamic and dependent on many factors, such as subcellular location of miRNAs, the abundancy of miRNAs and target mRNAs, and the affinity of miRNA-mRNA interactions. miRNA sequence (typically-22 base pairs) is partially complementary to binding sites in the 3′UTR of genes, often with full complementarity to 7 or 8 nucleotides in the “seed region” (residues 2-9) of the miRNA. Gene suppression in mammals is thought to occur primarily by inhibiting translation. However, miRNAs in mammals also cause mRNA decay. The miRNA target genes identified to date have mostly been involved in vital biological processes such as development, apoptosis, neural patterning, and viral infection. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

Nucleic acid molecules useful in the methods of the disclosure include, but are not limited to, any nucleic acid molecule with activity or that encodes a polypeptide. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” refers to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison. It will be understood that the numbering of the specific positions or residues in the respective sequences depends on the particular protein or polypeptide and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

A polypeptide (e.g., protein, or peptide) or nucleic acid (e.g., transcript, for example, mRNA, miRNA, siRNA etc.) may be represented as “substantially expressed” in a cell or tissue, which may refer to the level of expression of the polypeptide, or the nucleic acid, wherein the expression level is substantial, e.g., easily detectable and/or at least within a comparable range relative to expression in another reference cell or tissue. The expression “substantially” as in “substantially expressed” may mean in one aspect that the expression is detectable or the element is detectably expressed. In some cases substantially expressed is relative to another where it is not substantially expressed, whereas the former has higher expression than the latter, wherein the latter refers to where an element is not substantially expressed may not be detectable by ordinary experimental procedures known in the art or relevant to the context. In some cases, the expression may be at least 10 times higher when it is referred to as “substantially expressed” compared to a reference expression level that may be within a detectable range (but low), and that is not substantially expressed.

The term “subject” or “patient” may refer to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The term “therapeutic effect” may refer to some extent of relief of one or more of the symptoms of a disorder (e.g., a neoplasia, tumor, or infection by an infectious agent or an autoimmune disease) or its associated pathology. “Therapeutically effective amount” as used herein may refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

5′- and 3′-Untranslated Regions (UTRs), 5′-CAP and 3′ Poly A Tail of an mRNA

In one aspect an engineered polynucleotide construct for non-viral gene delivery and expression is disclosed herein, including factors that could potentially affect mRNA stability and translation efficiency, for example, the features related to a 5′ untranslated region (5′ UTR), a 3′ UTR, and a polyadenylation (Poly A tail). The untranslated regions at the 5′- and the 3′-end flanking the coding sequence are critical for expression of the encoded polypeptide when introduced into a cell. Of these, the 5′-UTR is responsible for efficient assembly of the ribosome on the translational start codon. In some embodiments, the engineered polynucleotide comprises an engineered untranslated region (UTR).

Naturally occurring mRNAs contain a 5′ cap structure which helps stabilize the RNA and is fundamental to eukaryotic gene expression (Shuman, S. et al Mol. Microbiol. 1995, 17, 405-410). These mRNAs also contain a 3′ poly A tail. Both modifications stabilize the mRNA encoding the protein. The 5′-end modification can comprise a cap structure based on guanosine or purine that increase expression of products (e.g., protein) encoded by the RNA, lower immunogenicity and/or improve stability of the RNA. The 3′-end modifications can comprise modification of the cis-diol at the 3′ and 2′ positions of the terminal ribose of the RNA, for example by inserting a ring atom between these positions or replacing one or both hydroxyl groups with other substituents, to improve stability of the RNA (e.g., by slowing the rate of degradation). The 5′-cap structure found at the 5′ end of eukaryotic messenger RNAs (mRNAs) and many viral RNAs consists of a N⁷-methylguanosine nucleoside linked to the 5′-terminal nucleoside of the pre-mRNA via a 5′-5′ triphosphate linkage. This cap structure fulfills many roles that ultimately lead to mRNA translation. RNA capping is also important for other processes, such as RNA splicing and export from the nucleus and to avoid recognition of mRNA by the cellular innate immunity machinery (Daffis, S. et al Nature 2010, 468, 452-6; Zist, R. et al Nat. Immunol. 2011, 12, 137-43).

The 3′ UTR can determine the half-life of an mRNA in a cell. mRNA degradation is regulated through protein complexes binding to regions within the 3′ UTR and the poly A tail. For example, regulatory sequences in the 3′-UTR containing adenylate-uridylate-rich elements (AU-rich elements; AREs) can destabilize mRNAs. The most important regulatory elements involved are the poly A tail and other cis-elements but multiple and diversified regulatory mechanisms have been described (Oktaba et al., 2015; Yue et al., 2018).

miRNA and miRNA Target Sequences for Negative Regulation of Expression in Non-Myeloid Cell Types

The present disclosure provides systems (including methods and compositions) for regulating an mRNA expression in a cell that is not the desired “target cell” for the expression of the mRNA. In some embodiments, this may be achieved by careful inclusion of miRNA target regions in the mRNA, the expression of which is meant to be regulated. Canonical miRNA binding involves base pairing of the miRNA seed region (nucleotides 2-8) to complementary target sites. In order to harness the effect of one or more miRNAs specific to a cell type, it would be necessary to have identified the miRNA target sequences of the miRNA seed region specific for the miRNA. Additionally, it is necessary to have determined the miRNAs that are specific to the cell type that is not the desired cell type, to use the miRNAs to regulate the expression of the mRNA. In some embodiments, the mRNA is a recombinant mRNA that comprises one or more miRNA target sites, such that a cell expressing an miRNA which interacts with the miRNA target site can cause the mRNA to decay or prevents the translation of the mRNA. In one embodiment, the current disclosure is about expressing a nucleic acid sequence encoding a polypeptide from a recombinant nucleic acid in a specific target cell, in a situation, for example, where the recombinant nucleic acid is introduced systemically in an organism and at the same time, avoiding the expression of the encoded polypeptide in a cell that is not the specific target cell (non-target cell or not the “desired” cell type). In one embodiment, an accidental uptake of a recombinant mRNA comprising a sequence encoding the polypeptide by a cell in which the recombinant mRNA is not desired for expression may be subjected to a nullifying effect of the uptake by adopting a measure in the design of the recombinant mRNA to include miRNA target sites in the recombinant mRNA that would allow the naturally occurring miRNA in the undesired cell type to degrade the recombinant mRNA or to prevent translation of the protein that is encoded by a sequence within the recombinant mRNA. In this approach, a negative regulation or a negative control for expression of the recombinant mRNA is including the miRNA target sites for one or more miRNAs that are prevalent in the undesired cell types but are absent in the cell type where the recombinant mRNA is desired to be expressed.

In one embodiment, the desired cell type for expressing the sequence is a myeloid cell, for example a macrophage or monocyte cell. In some embodiments the myeloid cell is a dendritic cell. In some embodiments, the myeloid cell is a CD14+ cell. In one embodiment, the undesired cell is an epithelial cell. In some embodiments, the undesired cell is a hepatocyte. In some embodiments, the desired cell is a myeloid cell, wherein a myeloid cell for the purposes of the present description is a monocyte derived macrophage, a monocyte, a dendritic cell and all the above combined, describing cells in which the recombinant nucleic acid is desired to be expressed. On many occasions, an exogenous nucleic acid gets taken up in abundant cell type and produces off-target effect. This measure allows minimization of off-target effects of the recombinant nucleic acid administered. Epithelial cells, hepatocytes and smooth muscle cells appear abundantly in living tissues. Therefore, systemically administered recombinant nucleic acids may produce off-target effects in these cells when the recombinant nucleic acids are desired to be expressed in specific cell type other than these cells.

Candidate miRNAs for which miRNA target sequences are incorporated in the mRNA constructs for selective expression of the mRNA in myeloid cells, and relative destruction or suppression of expression in non-myeloid cells should have at least 10-fold higher expression in the non-myeloid cells, compared to myeloid cells. More consistently the expression of the candidate miRNAs is negligible, or undetectable in myeloid cells. For the purposes of the present description, a relative expression level of a candidate miRNA is at least 10-fold less in a myeloid cell compared to a non-myeloid cell. For example, the candidate miRNA is at least 10, 10.1, 10.2, 10.3, 10.4, 10.5, 10.6, 10.7, 10.8, 10.9, 11, 12, 13, 14, 15, 16, 17, 18, 19, 20, 21, 22, 23, 24, 25, 26, 27, 28, 29, 30, 40, 50, 60, 70, 80, 90 or 100-fold more in a non-myeloid cell, e.g., a hepatocyte than in a myeloid cells, such as a monocyte derived myeloid cell.

In some embodiments, the miRNA prevalent in an undesired cell type is miR-192b. In some embodiments, the miRNA prevalent in a undesired cell type is miR-27b. In some embodiments, the miRNA prevalent in an undesired cell type is miR-10a. In some embodiments, the recombinant mRNA comprises an miRNA target site for an miRNA that is highly prevalent in a hepatocytes and epithelial cells and is absent in myeloid cells. For example, in some embodiments, the miRNA is hsa-miR-192b (abbreviated miR-192b). For example, in one embodiment, the miRNA is hsa-miR-27b (abbreviated miR-27b). For example, in one embodiment, the miRNA is hsa-miR-Oa (abbreviated miR-Oa).

According to studies in the literature, miR-192 is highly expressed in hepatocytes and epithelial cells, absent in myeloid cells. Cellular ontology information for miR-192 indicate significantly high expression of the microRNA in endodermal cells, epithelial cells and hepatocytes from human donor samples. Similarly, miR-27b is highly expressed in hepatocytes, endodermal cells and endo-epithelial cells. CD14+ myeloid cells, e.g., monocytes, macrophages and dendritic cells show very low to almost negligible expression in the human donors, compared to non-myeloid cells such as epithelial cells or hepatocytes. For example, in one study, dendritic cells derived from multiple donors show lack of expression of miR-27b and miR-192b. For example, monocyte derived macrophages from donor 1 and donor 2 show an expression level of miR-192b at 179 and 266 arbitrary units respectively; CD14+ monocytes from donor 1 show an expression level of miR-192b at 1,355 arbitrary units; CD14+ monocytes from donor 2 show an expression level of miR-192b at 1,644 arbitrary units. Comparing with non-myeloid cells: miR-192b expression in CD8+ T cells from donor 1 was 6,099 arbitrary units; in CD8+ T cells from donor 2 was 6,547 arbitrary units; in CD8+ T cells from donor 3 was 13,812 arbitrary units; in CD4+ T cells from donor 1 was 6,894 arbitrary units; in CD4+ T cells from donor 2 was 6,850 arbitrary units; in CD19+ B cells from donor 1 was 6,894 arbitrary units; in CD19+ B cells from donor 2 was 6,460 arbitrary units; in NK cells from donor 2 was 11,304 arbitrary units; in NK cells from donor 3 was 11,536 arbitrary units; in hepatocytes from donor 1 was 278,282 arbitrary units; in hepatocytes from donor 2 was 238,786 arbitrary units; and in intestinal epithelial cells from donor 1 was 303,408. miR-192b therefore appears to be a good candidate for suppressing expression of the recombinant polynucleic acid-encoded protein in hepatocytes and epithelial cells, while expression of the same would remain unaffected in the myeloid cells. By naturally occurring miR-mediated negative regulation of expression of the recombinant nucleic acid in cells that are not the desired cells, a cell selective expression of the recombinant nucleic acid can be achieved.

Expression of miR-27b in CD14+ monocytes from donor 1 is 5,154 arbitrary units, in donor 2 is 5119 arbitrary units, and in donor 3 is 4492 arbitrary units; monocyte derived macrophages from donor 1 is 2,143; from donor 2 is 718; from donor 3 is 1,721 arbitrary units. In comparison, expression in hepatocytes were 91,758; 99,780 and 94, 187 in the three donors respectively. miR-27b therefore appears to be a good candidate for suppressing expression of the recombinant polynucleic acid-encoded protein in hepatocytes and epithelial cells, while expression of the same would remain unaffected in the myeloid cells.

Expression of miR-10a appears to be significantly higher in many epithelial cell types, hepatic sinusoidal endothelial cells and muscle cells, while being expressed at very low to almost insignificant levels in dendritic cells, monocyte derived macrophages and CD14+ monocytes. For example, expression of miR-10a in CD14+ monocytes are 1,258 and 939 arbitrary units from donor 1 and donor 2 samples respectively; monocyte derived macrophages from donor 1 show expression level of 2,127 arbitrary units, from donor 2 was 316 arbitrary units, and from donor 3 was 1303 arbitrary units. Compared to those, the expression in renal epithelial cells was 242,330; in renal proximal tubular epithelial cells was 222,685; in renal glomerular endothelial cells was 125,314; and in hepatic sinusoidal epithelial cells was 140,929 arbitrary units from donor 1; and likewise, cells from donor 2 showed high expression of miR-10a in epithelial, hepatic and smooth muscle cells. For example, in renal proximal tubular epithelial cells the expression was 207,925 and renal mesangial cells was 180,568 arbitrary units from donor 2 samples. In colonic smooth muscle cells from donor 1 was 171,804 arbitrary units and from donor 2 was 209,172 arbitrary units. These indicate that miR-10a appears to be a good candidate for suppressing expression of the recombinant polynucleic acid-encoded protein in non-myeloid cells such as hepatocytes and epithelial and smooth muscle cells, while expression of the same would remain unaffected in the myeloid cells.

In some embodiments, the exemplary human microRNA miR-192 (NR_029578) 110 bp RNA

(SEQ ID NO: 1)
GCCGAGACCG AGTGCACAGG GCTCTGACCT ATGAATTGAC
AGCCAGTGCT CTCGTCTCCC CTCTGGCTGC CAATTCCATA
GGTCACAGGT ATGTTCGCCT CAATGCCAGC.

In some embodiments, the exemplary human microRNA is miR-27b (NR_029665) 97 bp is:

(SEQ ID NO: 2)
ACCTCTCTAA CAAGGTGCAG AGCTTAGCTG ATTGGTGAAC
AGTGATTGGT TTCCGCTTTG TTCACAGTGG CTAAGTTCTG
CACCTGAAGA GAAGGTG.

In some embodiments, the exemplary human microRNA miR-10a (NR_029608) 110 bp RNA

(SEQ ID NO: 3)
GATCTGTCTG TCTTCTGTAT ATACCCTGTA GATCCGAATT
TGTGTAAGGA ATTTTGTGGT CACAAATTCG TATCTAGGGG
AATATGTAGT TGACATAAAC ACTCCGCTCT.

In one embodiment, the recombinant mRNA may be designed to comprise one or more miRNA targets, (e.g., targets for one or more different miRNAs). In one embodiment, the recombinant mRNA may be designed to comprise 1, 2, 3, 4, 5, 6, 7, 8, 9, 10 or more miRNA targets for 1, 2, 3, 4, 5, 6, 7, 8, 9, 10 or more different miRNAs respectively. In some embodiments, the recombinant mRNA may be designed to comprise at least miRNA targets for at least 2 miRNAs. In some embodiments, the recombinant mRNA may be designed to comprise at least miRNA targets for at least 3 miRNAs.

In some embodiments, the recombinant mRNA comprises an miRNA target site at a 5′-UTR site of the recombinant mRNA, hereby designated as miRNA-5p. In some embodiments, the recombinant mRNA comprises an miRNA target site at a 3′-UTR site of the recombinant mRNA, hereby designated as miRNA-3p. In some embodiments, the recombinant mRNA comprises an miRNA target site at a 5′-UTR and an miRNA target site at a 3′-UTR site of the recombinant mRNA. In some embodiments, the recombinant mRNA comprises one or more miRNA target sites at a 5′-UTR and one or more miRNA target sites at a 3′-UTR site of the recombinant mRNA.

In some embodiments, the recombinant mRNA comprises a first miRNA target site in the 5′-UTR for miR-192b, and a second target site in 3′UTR for miR-27b and/or miR-10a. In some embodiments, the recombinant mRNA comprises a first miRNA target site in the 5′-UTR for miR-192b (e.g., miR-192b-5p), a second target site in 3′UTR for miR-27b (e.g., miR-27b-3p) and a third target site in 3′UTR for miR-10a (e.g., miR-10a-3p). In some embodiments, the recombinant mRNA comprises a first miRNA target site in the 5′-UTR for miR-27b, and a second target site in 3′UTR for miR-192b and/or miR-10a. In some embodiments, the recombinant mRNA comprises a first miRNA target site in the 5′-UTR for miR-27b (e.g., miR-27b-5p), a second target site in 3′UTR for miR-192b (e.g., miR-192b-3p) and a third target in 3′UTR site for miR-10a (e.g., miR-10a-3p). In some embodiments, the recombinant mRNA comprises a first miRNA target site in the 5′-UTR for miR-10a (e.g., miR-10a-5p), a second target site in 3′UTR for miR-192b (e.g., miR-192b-3p) and a third target in 3′UTR site for miR-27a (e.g., miR-27b-3p).

In some embodiments, the recombinant mRNA comprises a first miRNA target site in the 5′-UTR for miR-27b, and a second target site in 3′UTR for miR-192b and/or miR-10a. In some embodiments, the recombinant mRNA comprises a first miRNA target site in the 5′-UTR for miR-27b (e.g., miR-27b-5p), a second target site in 3′UTR for miR-192b (e.g., miR-192b-3p) and a third target in 3′UTR site for miR-10a (e.g., miR-10a-3p). In some embodiments, the recombinant mRNA comprises a first miRNA target site in the 5′-UTR for miR-10a (e.g., miR-10a-5p), a second target site in 3′UTR for miR-192b (e.g., miR-192b-3p) and a third target in 3′UTR site for miR-27a (e.g., miR-27b-3p).

In some embodiments, the recombinant mRNA comprises a first miRNA target site in the 5′-UTR for miR-192b (e.g., miR-192-5p) and a second target site in the 5′-UTR for miR-27b (e.g., miR-27b-5p), and a third target site in 3′UTR for miR-10a (e.g., miR-10a-3p).

In some embodiments, the recombinant mRNA comprises a first miRNA target site in the 5′-UTR for miR-27b (e.g., miR-27b-5p) and a second target site in the 5′-UTR for miR-10a (e.g., miR-10a-5p), and a third target site in 3′UTR for miR-192b (e.g., miR-192b-3p).

In some embodiments, the recombinant mRNA comprises a first miRNA target site in the 5′-UTR for miR-192b (e.g., miR-192b-5p) and a second target site in the 5′-UTR for miR-10a (e.g., miR-10a-5p), and a third target site in 3′UTR for miR-27b (e.g., miR-27b-3p).

In some embodiments there are two, three or more miRNA target sequences in the 5′UTR of the recombinant mRNA.

In some embodiments there are two, three or more miRNA target sequences in the 3′UTR of the recombinant mRNA.

In one aspect a method of expressing an exogenous polypeptide is provided, wherein a recombinant nucleic acid is contacted to a heterogenous population of cells comprising a myeloid cell and a non-myeloid cell and wherein expression of the exogenous polypeptide encoded by the recombinant nucleic acid occurs in the myeloid cell and does not occur in the non-myeloid cells of the heterogenous population of cells. The method comprises contacting the heterogenous population of cells with a composition comprising the recombinant polynucleic acid, wherein the recombinant nucleic acid comprises (a) a sequence encoding the exogenous polypeptide, and (b) one or more miRNA target sequences described above; wherein the exogenous polypeptide is expressed in the myeloid cell of the heterogenous population of cells, and wherein the exogenous polypeptide is not substantially expressed in the non-myeloid cells of the heterogenous population of cells. In some embodiments, the exogenous polypeptide is expressed at a level that is at least 10-fold higher in the myeloid cell than in the non-myeloid cells of the heterogenous population of cells. In some embodiments, the exogenous polypeptide that is not substantially expressed in the non-myeloid cells may refer to the exogenous polypeptide being expressed at least 10-fold less in the non-myeloid cells compared to the myeloid cells of the heterogenous population of cells. In some embodiments, the exogenous polypeptide is expressed at least 10-fold to 100-fold higher level in the myeloid cells than in the non-myeloid cells of the heterogenous population of cells. In some embodiments, the expression of the exogenous polypeptide is undetectable in the non-myeloid cells of the heterogenous population of cells. In some embodiments, the recombinant nucleic acid is mRNA.

In one aspect, provided herein is a method of expressing an exogenous polypeptide from a recombinant nucleic acid in a myeloid cell, the method comprising contacting a heterogenous population of cells comprising the myeloid cell and a non-myeloid cell with a composition comprising the recombinant polynucleic acid, wherein the recombinant nucleic acid comprises (a) a sequence encoding the exogenous polypeptide, (b) a sequence comprising (i) one or more miRNA target sequences, and (ii) a sequence encoding a polypeptide required for expression of the exogenous polypeptide; wherein the exogenous polypeptide is expressed in the myeloid cell of the heterogenous population of cells, and wherein the exogenous polypeptide encoded by the sequence of the recombinant nucleic acid is not substantially expressed in the non-myeloid cells of the heterogenous population of cells. In some embodiments, the polypeptide required for expression of the exogenous polypeptide is a polypeptide that is a regulator, e.g., an enhancer, a transcription inducer or a transcription repressor, an inducer for an inducible promoter of the exogenous polypeptide that is expressed in the myeloid cell; miRNA mediated suppression of expression of such a polypeptide would result in lack of expression of the exogenous polypeptide. In some embodiments the inducible promoter is operably linked with the sequence encoding the exogenous polypeptide. For example, an inducible promoter is a tetracycline inducible promoter. In some embodiments the recombinant nucleic acid is DNA or RNA. In some embodiments the RNA is a polycistronic RNA. In some embodiments, the DNA is a plasmid DNA.

In one aspect, provided herein is a method of expressing an exogenous polypeptide from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the exogenous polypeptide, and (b) one or more miRNA target sequences; and wherein the recombinant nucleic acid lacks a sequence encoding a viral protein or portion thereof, and wherein the exogenous polypeptide is expressed in the cell that is the first cell type of the heterogenous population of cells, and wherein the exogenous polypeptide is not substantially expressed in the cell that is the second cell type of the heterogenous population of cells. In some embodiments, the first cell type is a myeloid cell type is a myeloid cell type, and the second cell type is a non-myeloid cell type. In some embodiments, the non-myeloid cell of the heterogenous population of cells comprises any one of a hepatocyte, a lymphoid cell (e.g., T cell, B cell), an alveolar cell, a cardiac cell, a kidney cell, a stromal cell, an epithelial cell, a muscle cell, an ocular cell, a nerve cell, a brain cell and a skeletal cell. A myeloid cell may be for example, a macrophage, a monocyte, a dendritic cell, a monocyte derived cell, or a CD14+ cell.

In one aspect, provided herein is a method of expressing an exogenous polypeptide from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the exogenous polypeptide, (b) a sequence comprising (i) one or more miRNA target sequences, and (ii) a sequence encoding a polypeptide required for expression of the exogenous polypeptide; wherein the composition lacks a sequence encoding a viral protein or portion thereof, and wherein the exogenous polypeptide is expressed in the cell that is the first cell type of the heterogenous population of cells, and wherein the exogenous polypeptide is not substantially expressed in the cell that is the second cell type of the heterogenous population of cells.

In one aspect, provided herein is a method of expressing a chimeric fusion protein from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the chimeric fusion protein, and (b) one or more miRNA target sequences; and wherein the chimeric fusion protein is expressed in the cell that is the first cell type of the heterogenous population of cells, and wherein the chimeric fusion protein is not substantially expressed in the cell that is the second cell type of the heterogenous population of cells. In some embodiments, the first cell type is a myeloid cell type. In some embodiments, the second cell type is a non-myeloid cell type. In some embodiments, the non-myeloid cell type comprises an alveolar cell, a cardiac cell, a kidney cell, a stromal cell, an epithelial cell, a muscle cell, an ocular cell, a nerve cell, a brain cell, a skeletal cell or a hepatocyte.

In one aspect, provided herein is a method for expressing a chimeric fusion protein from a recombinant nucleic acid in a first cell type, the method comprising contacting a heterogenous population of cells comprising a cell that is the first cell type and a cell that is a second cell type with a composition comprising the recombinant polynucleic acid, wherein the recombinant nucleic acid comprises (a) a sequence encoding the exogenous polypeptide, (b) a sequence comprising (i) one or more miRNA target sequences, and (ii) a sequence encoding a polypeptide required for expression of the exogenous polypeptide; wherein the chimeric fusion protein is expressed in the cell that is the first cell type of the heterogenous population of cells, and wherein the chimeric fusion protein is not substantially expressed in the cell that is the second cell type of the heterogenous population of cells. In one aspect, the first cell type is a myeloid cell type. In some embodiments, the second cell type is a non-myeloid cell type.

In some embodiments, one or more target regulating factors may be co-expressed. Exemplary miRNA target regulating factors can include siRNA, shRNA, and/or miRNA. In some embodiments the siRNA, shRNA and/or miRNA targets RNA. In some embodiments, the mixture comprises a RNA mimic, which is a sequence that is analogous to another RNA sequence. In some embodiments the mixture comprises a small molecule agent or moiety that regulates the levels of the target miRNA. In some embodiments, these components will be administered together. In some embodiments, these components may be administered separately.

In some embodiments, expression of the polypeptide encoded by the recombinant nucleic acid as designed above occurs in a myeloid cell; whereas, the same is not substantially expressed in the non-myeloid of the heterogenous population of cells. In some embodiments, expression of a polypeptide from the recombinant nucleic acid as designed above occurs is not substantially expressed when the expression is at least 10 times lower in a second cell compared to a first cell, where it is substantially expressed. In some embodiments, expression of a polypeptide from the recombinant nucleic acid having the miRNA target sequences is at least 10 times lower in the undesired cells that express the miRNA, compared to a desired cell in which the miRNA is absent. In some embodiments, the expression of a polypeptide from the recombinant nucleic acid having the miRNA target sequences is at least 10, 12, 15, 20, 25, 30, 35, 40, 45, 50, 55, 60, 65, 70, 75, 80, 85, 90, 95, 100 or more times lower in the undesired cells that express the miRNA, compared to a desired cell in which the miRNA is absent. In some embodiments, the expression of a polypeptide from the recombinant nucleic acid having the miRNA target sequences is undetectable in the undesired cell types.

In some embodiments, enhancement of expression of a polypeptide encoded by a polynucleotide may be achieved using non-native or synthetic 5′- and 3′ UTRs. In some embodiments, recombinant polynucleotides are engineered with non-native or synthetic 5′- and 3′-UTRs that greatly enhance the expression of a protein coding sequence comprised in a nucleic acid when introduced in a myeloid cell. In some embodiments, the 5′ UTR may be encoded within a plasmid in which the DNA sequence encoding an exogenous protein coding sequence is incorporated for delivery and expression. Likewise, in some embodiments, the 3′ UTR may also be encoded within a plasmid in which the DNA sequence encoding an exogenous protein coding sequence is incorporated for delivery and expression. In some embodiments, the nucleic acid is an IVT RNA. In some embodiments the 5′- and/or the 3′ UTR may be added to the mRNA comprising the sequence encoding an exogenous protein in vitro in an enzymatic reaction. A poly A tail can be added enzymatically in vitro to an RNA or can be an encoded poly A tail, such as by an IVT template. In some embodiments, enzymatically added poly A tails can be tailored to have a desired length.

A proper 5′-cap structure is important in the synthesis of functional messenger RNA. In some embodiments, the 5′-cap comprises a guanosine triphosphate arranged as GpppG at the 5′terminus of the nucleic acid.

In some embodiments, the mRNA comprises a 5′ 7-methylguanosine cap, m7-GpppG. A 5′ 7-methylguanosine cap can increase mRNA translational efficiency and prevents degradation of mRNA 5′-3′exonucleases. In some embodiments, the mRNA comprises “anti-reverse” cap analog (ARCA, m7,3′-O GpppG).

In some embodiments, the guanosine cap is a Cap 0 structure.

In some embodiments, the guanosine cap is a Cap 1 structure.

In addition to its essential role of cap-dependent initiation of protein synthesis, the mRNA cap can also function as a protective group from 5′ to 3′ exonuclease cleavage and a unique identifier for recruiting protein factors for pre-mRNA splicing, polyadenylation and nuclear export. It acts as the anchor for the recruitment of initiation factors that initiate protein synthesis and the 5′ to 3′ looping of mRNA during translation. Three enzymatic activities are required to generate the Cap 0 structure, namely, RNA triphosphatase (TPase), RNA guanylyltransferase (GTase) and guanine-N7 methyltransferase (guanine-N7 MTase). Each of these enzyme activities carries out an essential step in the conversion of the 5′ triphosphate of nascent RNA to the Cap 0 structure. RNA TPase removes the γ-phosphate from the 5′ triphosphate to generate 5′ diphosphate RNA. GTase transfers a GMP group from GTP to the 5′ diphosphate via a lysine-GMP covalent intermediate. The guanine-N7 MTase then adds a methyl group to the N7 amine of the guanine cap to form the cap 0 structure. For Cap 1 structure, m7G-specific 2′O methyltransferase (2′O MTase) methylates the +1 ribonucleotide at the 2′O position of the ribose to generate the cap 1 structure. The nuclear RNA capping enzyme interacts with the polymerase subunit of RNA polymerase II complex at phosphorylated Ser5 of the C-terminal heptad repeats. RNA guanine-N7 methyltransferase also interacts with the RNA polymerase II phosphorylated heptad repeats. In some embodiments, the cap is a G-quadruplex cap. In some embodiments, the 5′ Cap is added to the 5′ end of the mRNA by co-transcriptional capping, in which, a cap analog is introduced into the transcription reaction, along with the four standard nucleotide triphosphates, in an optimized ratio of cap analog to GTP 4:1. This allows initiation of the transcript with the cap structure in a large proportion of the synthesized RNA molecules. This approach produces a mixture of transcripts, of which ~80% are capped, and the remainder have 5′triphosphate ends. In some embodiments, the cap analogs used in co-transcriptional RNA capping is the standard 7-methyl guanosine (m7G) cap analog.

In some embodiments, the cap analogs used in co-transcriptional RNA capping is the anti-reverse cap analog (ARCA), also known as 3′ O-me 7-meGpppG cap analog. ARCA is methylated at the 3′ position of the m7G, preventing RNA elongation by phosphodiester bond formation at this position. Thus, transcripts synthesized using ARCA contain 5′-m7G cap structures in the correct orientation, with the 7-methylated G as the terminal residue. In contrast, the m7G cap analog can be incorporated in either the correct or the reverse orientation.

In one embodiment, the 5′ UTR comprises about 50 nucleotides. In some embodiments, the 5′ UTR comprises less than 50 nucleotides. In one embodiment, the 5′ UTR may comprise more than 50 nucleotides. In one embodiment, the 5′ UTR comprises at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 nucleotides.

In some embodiments, the 5′ UTR comprises is at least 45 nucleotides in length. In one embodiment, the 5′ UTR comprises a translation start site that is at least 15 nucleotides downstream of the 5′ end. In some embodiment, the 5′ UTR comprises is at least 45 nucleotides in length. In one embodiment, the 5′ UTR comprises a translation start site that is at least 20 nucleotides downstream of the 5′ end. In some embodiment, the 5′ UTR comprises is at least 45 nucleotides in length. In one embodiment, the 5′ UTR comprises a translation start site that is at least 25 nucleotides downstream of the 5′ end. In some embodiment, the 5′ UTR comprises is at least 45 nucleotides in length. In one embodiment, the 5′ UTR comprises a translation start site that is at least 30 nucleotides downstream of the 5′ end.

In some embodiments, the 5′ UTR comprises is at least 45 nucleotides in length. In one embodiment, the 5′ UTR comprises a single translational start site.

In some embodiments, addition of a poly-A tail at the end of transcription may be done enzymatically. For example, the poly A tailing may be performed using E. coli poly(A) polymerase. Alternatively, for example, the poly A tailing may be performed using Saccharomyces (yeast) poly(A) polymerase. The length of the poly A tail introduced can be optimized by titrating the reaction.

In some embodiments, the nucleic acid comprises about 100-250 poly adenosyl (poly (A)) residues.

In some embodiments, the poly A length is between 50-200 Adenosine nucleotide residues (SEQ ID NO: 27).

In some embodiments, the poly A length is at least about 120 Adenosine nucleotide residues.

In some embodiments, the poly A length is at least about 50, 60, 70, 80, 90, 100, 110, 120, 130, 140, 150, 160, 170, 180, 190, 200, 210, 220, 230, 240, or about 250 Adenosine residues.

In some embodiments the 5′ and 3′ UTRs for stabilizing the nucleic acid inside the cell and ensuring improved expression of the exogenous protein comprises any one of the sequences from from sequences described herein. In some embodiments, the cell is a myeloid cell. In some embodiments the cell is a myeloid precursor cell. In some embodiments, the cell is a CD14+ cell. In some embodiments the cell is a CD14+/CD16− cell.

In some embodiments, an mRNA having 50 nucleotides-20,000 nucleotides can be expressed in myeloid cells using the methods described herein. In some embodiments, the mRNA comprises a coding region of about 1000, 2000, 3000, 4000, 5000, 6000, 7000, 8000, 9000, 10,000, 11,000, 12,000, 13,000, 14,000 15,000, 16,000, 17,000, 18,000 19,000 or about 20,000 nucleotides.

In some embodiments, replacing the 5′ UTR and/or the 3′ UTR improves expression of the encoded protein or polypeptide by at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% at least 160%, at least 170%, at least 180%, at least 190%, at least 200% or more compared to standard UTRs.

In some embodiments, replacing the 5′ UTR and/or the 3′ UTR improves duration of expression of the encoded protein or polypeptide in the myeloid cell by at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% at least 160%, at least 170%, at least 180%, at least 190%, at least 200% or more compared to standard UTRs.

In some embodiments expression of the protein is detectable according to an immunoassay after at least 72 hours after (a). In some embodiments, expression of the protein is detectable in at least 10% to at least 50% of the cells according to an immunoassay after at least 72 hours after (a). In some embodiments, expression of the protein is detectable in at least 40% of the cells according to an immunoassay after at least 72 hours after (a). In some embodiments, expression of the protein is detectable in at least 30% of the cells according to an immunoassay after at least 72 hours after (a). In some embodiments, expression of the protein is detectable in at least 20% of the cells after at least 72 hours after (a). In some embodiments expression of the protein is detectable according to an immunoassay after at least 48 hours after (a). In some embodiments, expression of the protein is detectable in at least 10% to at least 90% of the cells according to an immunoassay after at least 48 hours after (a). In some embodiments, expression of the protein is detectable in at least 80% of the cells, at least 70% of the cells, at least 60% of the cells, at least 50% of the cells, at least 40% of the cells, at least 30% of the cells, or at least 20% of the cells according to an immunoassay after at least 48 hours after (a).

In some embodiments, expression of a protein encoded by the nucleic acid is detectable, such as by an immunoassay, up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after incorporation of the nucleic acid into the cell. In some embodiments, expression of a protein encoded by the nucleic acid is detectable, such as by an immunoassay, up to and/or at least 96 hours after incorporation of the nucleic acid into the cell. In some embodiments, expression of a protein encoded by the nucleic acid is detectable, such as by an immunoassay, up to and/or at least 3 days after incorporation of the nucleic acid into the cell. In some embodiments, expression of a protein encoded by the nucleic acid is detectable, such as by an immunoassay, up to and/or at least 4 days after incorporation of the nucleic acid into the cell. In some embodiments, expression of a protein encoded by the nucleic acid is detectable, such as by an immunoassay, up to and/or at least 5 days after incorporation of the nucleic acid into the cell. In some embodiments, expression of a protein encoded by the nucleic acid is detectable, such as by an immunoassay, up to and/or at least 6 days after incorporation of the nucleic acid into the cell. In some embodiments, expression of a protein encoded by the nucleic acid is detectable, such as by an immunoassay, up to and/or at least 7 days after incorporation of the nucleic acid into the cell. In some embodiments, expression of a protein encoded by the nucleic acid is detectable, such as by an immunoassay, up to and/or at least 8 days after incorporation of the nucleic acid into the cell. In some embodiments, expression of a protein encoded by the nucleic acid is detectable, such as by an immunoassay, up to and/or at least 9 days after incorporation of the nucleic acid into the cell. In some embodiments, expression of a protein encoded by the nucleic acid is detectable, such as by an immunoassay, up to and/or at least 10 days or more after incorporation of the nucleic acid into the cell. In some embodiments, expression of a protein encoded by the nucleic acid is detectable, such as by an immunoassay, in at least 10% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 20% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 30% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 40% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 50% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 60% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 70% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 80% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 90% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells.

In some embodiments, the target cell is a mammalian cell. In some embodiments, the target cell is a human cell. In some embodiments, the target cell comprises a cell infected with a pathogen. In some embodiments, the target cell is a cancer cell. In some embodiments, the target cell is a cancer cell that is a lymphocyte. In some embodiments, the target cell is a cancer cell that is an ovarian cancer cell. In some embodiments, the target cell is a cancer cell that is a breast cell. In some embodiments, the target cell is a cancer cell that is a pancreatic cell. In some embodiments, the target cell is a cancer cell that is a glioblastoma cell.

In some embodiments, the nucleic acid is DNA. In some embodiments, the nucleic acid is RNA. In some embodiments, the nucleic acid is mRNA. In some embodiments, the nucleic acid is an unmodified mRNA. In some embodiments, the nucleic acid is a modified mRNA. In some embodiments, the nucleic acid is a circRNA. In some embodiments, the nucleic acid is a tRNA. In some embodiments, the nucleic acid is a microRNA. In some embodiments, the nucleic acid is an engineered nucleic acid. In some embodiments, the nucleic acid is a recombinant nucleic acid. In some embodiments, the nucleic acid is an in vitro transcribed nucleic acid.

Also provided herein is a vector comprising a nucleic acid sequence encoding a CFP described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′ UTR and/or a 3′ UTR flanking the at least one nucleic acid sequence encoding one or more polypeptides. In some embodiments, the vector further comprises one or more regulatory regions.

Also provided herein is a polypeptide encoded by the nucleic acid of a composition described herein.

Also provided herein is a cell comprising a composition described herein, a vector described herein or a polypeptide described herein. In some embodiments, the cell is a phagocytic cell. In some embodiments, the cell is a stem cell derived cell, a myeloid cell, a macrophage, a dendritic cell, a lymphocyte, a mast cell, a monocyte, a neutrophil, a microglia, or an astrocyte. In some embodiments, the cell is an autologous cell. In some embodiments, the cell is an allogeneic cell. In some embodiments, the cell is an M1 cell. In some embodiments, the cell is an M2 cell. In some embodiments, the cell is an M1 macrophage cell. In some embodiments, the cell is an M2 macrophage cell. In some embodiments, the cell is an M1 myeloid cell. In some embodiments, the cell is an M2 myeloid cell.

mRNA Nucleotide Modifications

In some aspects, the oligonucleotide described herein comprises at least one chemical modification. A chemical modification can be a substitution, insertion, deletion, chemical modification, physical modification, stabilization, purification, or any combination thereof. In some cases, a modification is a chemical modification. In one embodiments, the modification can be a phosphonate modification. In one aspect, the phosphonate modification is a phosphorothioate (PS Rp isomer). In one aspect, the phosphonate modification is a phosphorothioate (PS Sp isomer).

In some aspects, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the oligonucleotide comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, the chiral phosphate product is present in a diastereomeric excess of 95%. In some aspects, the chiral phosphate product is present in a diastereomeric excess of 96%. In some aspects, the chiral phosphate product is present in a diastereomeric excess of 97%. In some aspects, the chiral phosphate product is present in a diastereomeric excess of 98%. In some aspects, the chiral phosphate product is present in a diastereomeric excess of 99%. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In one embodiments, the modification can be a ribose modification. In one aspect, the ribose modification is a 2′-O-Methyl (2′-OME) modification. In one aspect, the ribose modification is 2′-O-Methoxyethyl (2′-O-MOE) modification. In one aspect, the ribose modification is 2′-deoxy-2′-fluoro (2′-F). In one aspect, the ribose modification is 2′-arabino fluoro (2′-Ara-F). In one aspect, the ribose modification is 2′-O-benzyl. In one aspect, the ribose modification is 2′-O-methyl-4-pyridine (2′-O-CH2Py(4)). In one aspect the ribose modification is a locked nucleic acid (LNA). In one aspect, the ribose modification can be a base modification. In one aspect the base modification is pseudouridine (Ψ). In one aspect, the ribose modification is 2′-thiouridine (s2U). In one aspect, the ribose modification is N6′-methyladenosine (m⁶C). In one aspect, the ribose modification is 5′-methylcytidine (m⁵C). In one aspect, the ribose modification is 5′-fluoro-2′-dioxyuridine. In one aspect, the ribose modification is N-ethyl-piperidine (7′-EAA triazole modified adenine. In one aspect, the ribose modification is N-ethylpiperidine 6′ triazole modified adenine. In one aspect, the ribose modification is 6-phenylpyrrolocytosine, In one aspect, the ribose modification is 2′-4′-difluorotoluyl ribonucleoside (rF). In one aspect, the ribose modification is 5′-nitroindole.

In some aspects, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the nucleic acids described herein. The nucleobase of the nucleotide can be independently selected from a purine, a pyrimidine, a purine or pyrimidine analog. In some aspects, the nucleobase can be naturally occurring or synthetic derivatives of a base.

In some aspects, the chemical modification described herein comprises modifying an uracil. In some aspects, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, l-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some aspects, the chemical modification described herein comprises modifying a cytosine. In some aspects, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some aspects, the chemical modification described herein comprises modifying a adenine. In some aspects, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some aspects, the chemical modification described herein comprises modifying a guanine. In some aspects, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, O⁶-Methyl-2′-deoxyguanosine, 2′-F-ara-guanosine, and 2′-F-guanosine.

In some embodiments, the modification is in a terminal nucleotide.

In some embodiments, the modification is in an internal nucleotide.

In some embodiments, the mRNA is unmodified.

In some embodiments, the mRNA comprises 1, 2, or 3 or more modified nucleotides.

In some cases, a modification can be permanent. In other cases, a modification can be transient. In some cases, multiple modifications are made to the nucleic acid. The nucleic acid modification can alter physio-chemical properties of a nucleotide, such as their conformation, polarity, hydrophobicity, chemical reactivity, base-pairing interactions, or any combination thereof.

In some embodiments, a recombinant nucleic acid of the disclosure encodes a chimeric antigen receptor comprising an extracellular domain, a transmembrane domain and one or more intracellular signaling domain. In some embodiments, the one or more intracellular signaling domains comprise a phagocytosis signaling domain or phagocytosis enhancing domain, for example, the intracellular signaling domain comprises an intracellular signaling domain derived from a phagocytic receptor. In some embodiments, the phagocytosis signaling domain comprises an intracellular signaling domain derived from FcRγ, FcRα or FcRε. In some embodiments, the phagocytosis signaling domain comprises an intracellular signaling domain derived from CD3ζ. In some embodiments, the one or more intracellular signaling domains further comprises a proinflammatory signaling domain. In some embodiments, the proinflammatory signaling domain comprises a PI3-kinase (PI3K) recruitment domain.

In some embodiments, the CFP comprises: (a) an extracellular domain comprising: (i) a scFv that specifically binds CD5 (anti-CD5 binding domain), or an scFv that binds to HER2, or to TROP2, or to GPC3; and (ii) a hinge domain derived from CD8; a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRα, FcRγ or FcRε, and (ii) a second intracellular signaling domain: (A) comprising a PI3K recruitment domain, or (B) derived from CD40. In some embodiments, the CFP comprises as an alternative (c) to the above: an intracellular domain comprising at least two intracellular signaling domains, wherein the at least two intracellular signaling domains comprise: (i) a first intracellular signaling domain derived from a phagocytic receptor intracellular domain, and (ii) a second intracellular signaling domain derived from a scavenger receptor phagocytic receptor intracellular domain comprising: (A) comprising a PI3K recruitment domain, or (B) derived from CD40. Table 1A enlists an exemplary set of various domains and combinations thereof, that were used to design the CFPs described herein. In some embodiments, the CFP comprises and intracellular signaling domain derived from an intracellular signaling domain of an innate immune receptor.

In some embodiments, the polynucleic acid is an mRNA. In some embodiments, the polynucleic acid is a circRNA. In some embodiments, the polynucleic acid is a viral vector. In some embodiments, the polynucleic acid is delivered via a viral vector.

In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In one aspect, provided herein is a method of treating cancer in a human subject in need thereof comprising administering a pharmaceutical composition to the human subject, the pharmaceutical composition comprising: (a) a myeloid cell comprising a polynucleic acid sequence, wherein the polynucleic acid sequence comprises a sequence encoding a chimeric fusion protein (CFP), the CFP comprising: (i) an extracellular domain comprising an anti-CD5 binding domain, and (ii) a transmembrane domain operatively linked to the extracellular domain; and (b) a pharmaceutically acceptable carrier; wherein the myeloid cell expresses the CFP.

In some embodiments, upon binding of the CFP to CD5 expressed by a target cancer cell of the subject killing or phagocytosis activity of the myeloid cell is increased by greater than 20% compared to a myeloid cell not expressing the CFP. In some embodiments, growth of a tumor is inhibited in the human subject.

In some embodiments, the cancer is a CD5+ cancer. In some embodiments, the cancer is leukemia, T cell lymphoma, or B cell lymphoma.

In some embodiments, the anti-CD5 binding domain is a CD5 binding protein that comprises an antigen binding fragment of an antibody, an scFv domain, an Fab fragment, or an sdAb domain. In some embodiments, the anti-CD5 binding domain is a protein or fragment thereof that binds to CD5, such as a ligand of CD5 (e.g., a natural ligand of CD5).

In some embodiments, the CFP further comprises an intracellular domain, wherein the intracellular domain comprises one or more intracellular signaling domains, wherein the one or more intracellular signaling domains comprises a phagocytosis signaling domain and wherein a wild-type protein comprising the intracellular domain does not comprise the extracellular domain.

In some embodiments, the phagocytosis signaling domain comprises an intracellular signaling domain derived from a receptor other than Megf10, MerTk, FcRα and Bail. In some embodiments, the phagocytosis signaling domain comprises an intracellular signaling domain derived from FcRγ, FcRα or FcRε.

In some embodiments, the one or more intracellular signaling domains further comprises a proinflammatory signaling domains. In some embodiments, the proinflammatory signaling domain comprises a PI3-kinase (PI3K) recruitment domain. In some embodiments, the transmembrane domain comprises a CD8 transmembrane domain. In some embodiments, the extracellular domain comprises a hinge domain derived from CD8, a hinge domain derived from CD28 or at least a portion of an extracellular domain from CD68.

In some embodiments, the CFP comprises: (a) an extracellular domain comprising: (i) a scFv that specifically binds CD5, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FcRε, and (ii) a second intracellular signaling domain that: (A) comprises a PI3K recruitment domain, or (B) is derived from CD40. In some embodiments, the nucleic acid is mRNA or circRNA. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In some embodiments, the method further comprises administering an additional therapeutic agent selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity, an agent that promotes sequestration of lymphocytes in primary and/or secondary lymphoid organs, an agent that increases concentration of naïve T cells and central memory T cells in secondary lymphoid organs, and any combination thereof.

In some embodiments, the myeloid cell further comprises: (a) an endogenous peptide or protein that dimerizes with the CFP, (b) a non-endogenous peptide or protein that dimerizes with the CFP; and/or (c) a second recombinant nucleic acid sequence, wherein the second recombinant nucleic acid sequence comprises a sequence encoding a peptide or protein that interacts with the CFP; wherein the dimerization or the interaction potentiates phagocytosis by the myeloid cell expressing the CFP as compared to a myeloid cell that does not express the CFP.

In some embodiments, the myeloid cell exhibits (i) an increase in effector activity, cross-presentation, respiratory burst, ROS production, iNOS production, inflammatory mediators, extra-cellular vesicle production, phosphatidylinositol 3,4,5-trisphosphate production, trogocytosis with the target cell expressing the antigen, resistance to CD47 mediated inhibition of phagocytosis, resistance to LILRB1 mediated inhibition of phagocytosis, or any combination thereof; and/or (ii) an increase in expression of a IL-1, IL3, IL-6, IL-10, IL-12, IL-13, IL-23, TNFα, a TNF family of cytokines, CCL2, CXCL9, CXCL10, CXCL11, IL-18, IL-23, IL-27, CSF, MCSF, GMCSF, IL-17, IP-10, RANTES, an interferon, MHC class I protein, MHC class II protein, CD40, CD48, CD58, CD80, CD86, CD112, CD155, a TRAIL/TNF Family death receptor, TGFβ, B7-DC, B7-H2, LIGHT, HVEM, TL1A, 41BBL, OX40L, GITRL, CD30L, TIM1, TIM4, SLAM, PDL1, an MMP (e.g., MMP2, MMP7 and MMP9) or any combination thereof.

In some embodiments, the intracellular signaling domain is derived from a phagocytic or tethering receptor or wherein the intracellular signaling domain comprises a phagocytosis activation domain. In some embodiments, the intracellular signaling domain is derived from a receptor other than a phagocytic receptor selected from Megf10, MerTk, FcR-alpha, or Bail. In some embodiments, the intracellular signaling domain is derived from a protein, such as receptor (e.g., a phagocytic receptor), selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, a complement receptor, CR3, CR4, Tim-1, Tim-4 and CD169. In some embodiments, the intracellular signaling domain comprises a pro-inflammatory signaling domain. In some embodiments, the intracellular signaling domain comprises a pro-inflammatory signaling domain that is not a PI3K recruitment domain.

In some embodiments, the intracellular signaling domain is derived from an ITAM domain containing receptor.

In some embodiments, the intracellular signaling domain is derived from a phagocytic receptor other than a phagocytic receptor selected from Megf10, MerTk, FcRα, or Bail.

In some embodiments, the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, CR3, CR4, Tim-1, Tim-4 and CD169. In some embodiments, the intracellular signaling domain comprises a pro-inflammatory signaling domain.

In some embodiments, the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, CR3, CR4, Tim-1, Tim-4 and CD169. In some embodiments, the intracellular signaling domain comprises an intracellular signaling domain derived from a phagocytic receptor. In some embodiments, the intracellular signaling domain comprises an intracellular signaling domain derived from a phagocytic receptor other than a phagocytic receptor selected from Megf10, MerTk, FcRα, or Bail. In some embodiments, the intracellular signaling domain comprises an intracellular signaling domain derived from a phagocytic receptor selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CD3ζ, CR3, CR4, Tim-1, Tim-4 and CD169. In some embodiments, the intracellular signaling domain comprises a PI3K recruitment domain. In some embodiments, the intracellular signaling domain comprises an intracellular signaling domain derived from a scavenger receptor. In some embodiments, the intracellular domain comprises a CD47 inhibition domain. In some embodiments, the intracellular domain comprises a Rac inhibition domain, a Cdc42 inhibition domain or a GTPase inhibition domain. In some embodiments, the Rac inhibition domain, the Cdc42 inhibition domain or the GTPase inhibition domain inhibits Rac, Cdc42 or GTPase at a phagocytic cup of a cell expressing the PFP. In some embodiments, the intracellular domain comprises an F-actin disassembly activation domain, a ARHGAP12 activation domain, a ARHGAP25 activation domain or a SH3BP1 activation domain. In some embodiments, the intracellular domain comprises a phosphatase inhibition domain. In some embodiments, the intracellular domain comprises an ARP2/3 inhibition domain. In some embodiments, the intracellular domain comprises at least one ITAM domain. In some embodiments, the intracellular domain comprises at least 1, 2, 3, 4, 5, 6, 7, 8, 9, 10, or more ITAM domains. In some embodiments, the intracellular domain comprises at least one ITAM domain select from an ITAM domain of CD3 zeta, CD3 epsilon, CD3 gamma, CD3 delta, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain. In some embodiments, the intracellular domain comprises an F-actin depolymerization activation domain. In some embodiments, the intracellular domain lacks enzymatic activity.

In some embodiments, the intracellular domain does not comprise a domain derived from a CD3 zeta intracellular domain. In some embodiments, the intracellular domain does not comprise a domain derived from a MerTK intracellular domain. In some embodiments, the intracellular domain does not comprise a domain derived from a TLR4 intracellular domain. In some embodiments, the intracellular domain comprises a CD47 inhibition domain. In some embodiments, the intracellular signaling domain comprises a domain that activates integrin, such as the intracellular region of PSGL-1. In some embodiments, the intracellular signaling domain comprises a domain that activates Rap1 GTPase, such as that from EPAC and C3G. In some embodiments, the intracellular signaling domain is derived from paxillin. In some embodiments, the intracellular signaling domain activates focal adhesion kinase. In some embodiments, the intracellular signaling domain is derived from a single phagocytic receptor. In some embodiments, the intracellular signaling domain is derived from a single scavenger receptor. In some embodiments, the intracellular domain comprises a phagocytosis enhancing domain.

In some embodiments, the intracellular domain comprises a pro-inflammatory signaling domain. In some embodiments, the pro-inflammatory signaling domain comprises a kinase activation domain or a kinase binding domain. In some embodiments, the pro-inflammatory signaling domain comprises an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, a NLRP family member, NLRP1-14, NOD1, NOD2, Pyrin, AIM2, NLRC4, FCGR3A, FCERIG, CD40, Tank I-binding kinase (TBK), a caspase domain, a procaspase binding domain or any combination thereof.

In some embodiments, the intracellular domain comprises a signaling domain, such as an intracellular signaling domain, derived from a connexin (Cx) protein. For example, the intracellular domain can comprise a signaling domain, such as an intracellular signaling domain, derived from Cx43, Cx46, Cx37, Cx40, Cx33, Cx50, Cx59, Cx62, Cx32, Cx26, Cx31, Cx30.3, Cx31.1, Cx30, Cx25, Cx45, Cx47, Cx31.3, Cx36, Cx31.9, Cx39, Cx40.1 or Cx23. For example, the intracellular domain can comprise a signaling domain, such as an intracellular signaling domain, derived from Cx43.

In some embodiments, the intracellular domain comprises a signaling domain, such as an intracellular signaling domain, derived from a SIGLEC protein. For example, the intracellular domain can comprise a signaling domain, such as an intracellular signaling domain, derived from Siglec-1 (Sialoadhesin), Siglec-2 (CD22), Siglec-3 (CD33), Siglec-4 (MAG), Siglec-5, Siglec-6, Siglec-7, Siglec-8, Siglec-9, Siglec-10, Siglec-11, Siglec-12, Siglec-13, Siglec-14, Siglec-15, Siglec-16 or Siglec-17.

In some embodiments, the intracellular domain comprises a signaling domain, such as an intracellular signaling domain, derived from a C-type lectin protein. For example, the intracellular domain can comprise a signaling domain, such as an intracellular signaling domain, derived from a mannose receptor protein. For example, the intracellular domain can comprise a signaling domain, such as an intracellular signaling domain, derived from an asialoglycoprotein receptor protein. For example, the intracellular domain can comprise a signaling domain, such as an intracellular signaling domain, derived from macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin1) subfamily protein, dectin 1/CLEC7A, DNGR1/CLEC9A, Myeloid C type lectin like receptor (MICL) (CLEC12A), CLEC2 (CLEC1B), CLEC12B, a DC immunoreceptor (DCIR) subfamily protein, DCIR/CLEC4A, Dectin 2/CLEC6A, Blood DC antigen 2 (BDCA2) (CLEC4C), Mincle (macrophage inducible C type lectin) (CLEC4E), a NOD-like receptor protein, NOD-like receptor MHC Class II transactivator (CIITA), IPAF, BIRC1, a RIG-I-like receptor (RLR) protein, RIG-I, MDA5, LGP2, NAIP5/Birc1e, a NLRP protein, NLRP1, NLRP2, NLRP3, NLRP4, NLRP5, NLRP6, NLRP7, NLRP89, NLRP9, NLRP10, NLRP11, NLRP12, NLRP13, NLRP14, a NLR protein, NOD1 or NOD2, or any combination thereof.

In some embodiments, the intracellular domain comprises a signaling domain, such as an intracellular signaling domain, derived from a cell adhesion molecule. For example, the intracellular domain can comprise a signaling domain, such as an intracellular signaling domain, derived from an IgCAMs, a cadherin, an integrin, a C-type of lectin-like domains protein (CTLD) and/or a proteoglycan molecule. For example, the intracellular domain can comprise a signaling domain, such as an intracellular signaling domain, derived from an E-cadherin, a P-cadherin, a N-cadherin, a R-cadherin, a B-cadherin, a T-cadherin, or a M-cadherin. For example, the intracellular domain can comprise a signaling domain, such as an intracellular signaling domain, derived from a selectin, such as an E-selectin, an L-selectin or a P-selectin.

In some embodiments, the CFP does not comprise a full length intracellular signaling domain. In some embodiments, the intracellular domain is at least 5, 10, 20, 30, 40, 50, 60, 70, 80, 90, 100, 150, 200, 300, 300, 400, or 500 amino acids in length. In some embodiments, the intracellular domain is at most 10, 20, 30, 40, 50, 60, 70, 80, 90, 100, 150, 200, 300, 300, 400, or 500 amino acids in length.

In some embodiments, the nucleic acid encodes an FcRα chain extracellular domain, an FcRα chain transmembrane domain and/or an FcRα chain intracellular domain. In some embodiments, the nucleic acid encodes an FcRβ chain extracellular domain, an FcRβ chain transmembrane domain and/or an FcRβ chain intracellular domain. In some embodiments, the FcRα chain or the FcRβ chain forms a complex with FcRγ when expressed in a cell. In some embodiments, the FcRα chain or FcRβ chain forms a complex with endogenous FcRγ when expressed in a cell. In some embodiments, the FcRα chain or the FcRβ chain does not incorporate into a cell membrane of a cell that does not express FcRγ. In some embodiments, the CFP does not comprise an FcRα chain intracellular signaling domain. In some embodiments, the CFP does not comprise an FcRβ chain intracellular signaling domain. In some embodiments, the nucleic acid encodes a TREM extracellular domain, a TREM transmembrane domain and/or a TREM intracellular domain. In some embodiments, the TREM is TREM1, TREM 2 or TREM 3.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP.

A target cell is, for example, a cancer cell. In some embodiments, the engineered myeloid cell, after engulfment of a cancer cell may present a cancer antigen on its cell surface to activate a T cell. An “antigen” is a molecule capable of stimulating an immune response. Antigens recognized by T cells, whether helper T lymphocytes (T helper (TH) cells) or cytotoxic T lymphocytes (CTLs), are not recognized as intact proteins, but rather as small peptides that associate with MHC proteins (such as class I or class II MHC proteins) on the surface of cells. During the course of a naturally occurring immune response, antigens that are recognized in association with class II MHC molecules on antigen presenting cells (APCs) are acquired from outside the cell, internalized, and processed into small peptides that associate with the class II MHC molecules.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, -fold, 17-fold, 18-fold, 19-fold, 20-fold, 25-fold, 30-fold, 40-fold, 50-fold, 75-fold, or 100-fold compared to a cell not expressing the CFP.

In some embodiments, the target cell expressing the antigen is a cancer cell.

In some embodiments, a cell expressing the CFP exhibits an increase in phagocytosis of a target cell expressing the antigen compared to a cell not expressing the CFP. In some embodiments, a cell expressing the CFP exhibits at least a 1.1-fold increase in phagocytosis of a target cell expressing the antigen compared to a cell not expressing the CFP. In some embodiments, a cell expressing the CFP exhibits at least a 2-fold, 3-fold, 4-fold, 5-fold, 6-fold, 7-fold, 8-fold, 9-fold, 10-fold, 20-fold, 30-fold or 50-fold increase in phagocytosis of a target cell expressing the antigen compared to a cell not expressing the CFP. In some embodiments, a cell expressing the CFP exhibits an increase in production of a cytokine compared to a cell not expressing the CFP. In some embodiments, the cytokine is selected from the group consisting of IL-1, IL3, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, an interferon and combinations thereof. In some embodiments, a cell expressing the CFP exhibits an increase in effector activity compared to a cell not expressing the CFP. In some embodiments, a cell expressing the CFP exhibits an increase in cross-presentation compared to a cell not expressing the CFP. In some embodiments, a cell expressing the CFP exhibits an increase in expression of an MHC class II protein compared to a cell not expressing the CFP. In some embodiments, a cell expressing the CFP exhibits an increase in expression of CD80 compared to a cell not expressing the CFP. In some embodiments, a cell expressing the CFP exhibits an increase in expression of CD86 compared to a cell not expressing the CFP. In some embodiments, a cell expressing the CFP exhibits an increase in expression of MHC class I protein compared to a cell not expressing the CFP. In some embodiments, a cell expressing the CFP exhibits an increase in expression of TRAIL/TNF Family death receptors compared to a cell not expressing the CFP. In some embodiments, a cell expressing the CFP exhibits an increase in expression of B7-H2 compared to a cell not expressing the CFP. In some embodiments, a cell expressing the CFP exhibits an increase in expression of LIGHT compared to a cell not expressing the CFP. In some embodiments, a cell expressing the CFP exhibits an increase in expression of HVEM compared to a cell not expressing the CFP. In some embodiments, a cell expressing the CFP exhibits an increase in expression of CD40 compared to a cell not expressing the CFP. In some embodiments, a cell expressing the CFP exhibits an increase in expression of TL1A compared to a cell not expressing the CFP. In some embodiments, a cell expressing the CFP exhibits an increase in expression of 41BBL compared to a cell not expressing the CFP. In some embodiments, a cell expressing the CFP exhibits an increase in expression of OX40L compared to a cell not expressing the CFP. In some embodiments, a cell expressing the CFP exhibits an increase in expression of GITRL death receptors compared to a cell not expressing the CFP. In some embodiments, a cell expressing the CFP exhibits an increase in expression of CD30L compared to a cell not expressing the CFP. In some embodiments, a cell expressing the CFP exhibits an increase in expression of TIM4 compared to a cell not expressing the CFP. In some embodiments, a cell expressing the CFP exhibits an increase in expression of TIM1 ligand compared to a cell not expressing the CFP. In some embodiments, a cell expressing the CFP exhibits an increase in expression of SLAM compared to a cell not expressing the CFP. In some embodiments, a cell expressing the CFP exhibits an increase in expression of CD48 compared to a cell not expressing the CFP. In some embodiments, a cell expressing the CFP exhibits an increase in expression of CD58 compared to a cell not expressing the CFP. In some embodiments, a cell expressing the CFP exhibits an increase in expression of CD155 compared to a cell not expressing the CFP. In some embodiments, a cell expressing the CFP exhibits an increase in expression of CD112 compared to a cell not expressing the CFP. In some embodiments, a cell expressing the CFP exhibits an increase in expression of PDL1 compared to a cell not expressing the CFP. In some embodiments, a cell expressing the CFP exhibits an increase in expression of B7-DC compared to a cell not expressing the CFP. In some embodiments, a cell expressing the CFP exhibits an increase in respiratory burst compared to a cell not expressing the CFP. In some embodiments, a cell expressing the CFP exhibits an increase in ROS production compared to a cell not expressing the CFP. In some embodiments, a cell expressing the CFP exhibits an increase in iNOS production compared to a cell not expressing the CFP. In some embodiments, a cell expressing the CFP exhibits an increase in iNOS production compared to a cell not expressing the CFP. In some embodiments, a cell expressing the CFP exhibits an increase in extra-cellular vesicle production compared to a cell not expressing the CFP. In some embodiments, a cell expressing the CFP exhibits an increase in trogocytosis with a target cell expressing the antigen compared to a cell not expressing the CFP. In some embodiments, a cell expressing the CFP exhibits an increase in resistance to CD47 mediated inhibition of phagocytosis compared to a cell not expressing the CFP. In some embodiments, a cell expressing the CFP exhibits an increase in resistance to LILRB1 mediated inhibition of phagocytosis compared to a cell not expressing the CFP. In some embodiments, a cell expressing the CFP exhibits an increase in phosphatidylinositol 3,4,5-trisphosphate production.

In some embodiments, the extracellular domain of a CFP comprises an Ig binding domain. In some embodiments, the extracellular domain comprises an IgA, IgD, IgE, IgG, IgM, FcRγI, FcRγIIA, FcRγIIB, FcRγIIC, FcRγIIIA, FcRγIIIB, FcRn, TRIM21, FcRL5 binding domain. In some embodiments, the extracellular domain of a CFP comprises an FcR extracellular domain. In some embodiments, the extracellular domain of a CFP comprises an FcRα, FcRβ, FcRε or FcRγ extracellular domain. In some embodiments, the extracellular domain comprises an FcRα (FCAR) extracellular domain. In some embodiments, the extracellular domain comprises an FcRβ extracellular domain. In some embodiments, the extracellular domain comprises an FCER1A extracellular domain. In some embodiments, the extracellular domain comprises an FDGR1A, FCGR2A, FCGR2B, FCGR2C, FCGR3A, or FCGR3B extracellular domain. In some embodiments, the extracellular domain comprises an integrin domain or an integrin receptor domain. In some embodiments, the extracellular domain comprises one or more integrin α1, α2, αIIb, α3, α4, α5, α6, α7, α8, α9, α10, α11, αD, αE, αL, αM, αV, αX, β1, β2, β3, β4, β5, β6, β7, or β8 domains.

In some embodiments, the CFP further comprises an extracellular domain operatively linked to the transmembrane domain and the extracellular antigen binding domain. In some embodiments, the extracellular domain further comprises an extracellular domain of a receptor, a hinge, a spacer and/or a linker. In some embodiments, the extracellular domain comprises an extracellular portion of a phagocytic receptor. In some embodiments, the extracellular portion of the CFP is derived from the same receptor as the receptor from which the intracellular signaling domain is derived. In some embodiments, the extracellular domain comprises an extracellular domain of a scavenger receptor. In some embodiments, the extracellular domain comprises an immunoglobulin domain. In some embodiments, the immunoglobulin domain comprises an extracellular domain of an immunoglobulin or an immunoglobulin hinge region. In some embodiments, the extracellular domain comprises a phagocytic engulfment domain. In some embodiments, the extracellular domain comprises a structure capable of multimeric assembly. In some embodiments, the extracellular domain comprises a scaffold for multimerization. In some embodiments, the extracellular domain is at least 10, 20, 30, 40, 50, 60, 70, 80, 90, 100, 150, 200, 300, 300, 400, or 500 amino acids in length. In some embodiments, the extracellular domain is at most 500, 400, 300, 200, or 100 amino acids in length. In some embodiments, the extracellular antigen binding domain specifically binds to the antigen of a target cell. In some embodiments, the extracellular antigen binding domain comprises an antibody domain. In some embodiments, the extracellular antigen binding domain comprises a receptor domain, antibody domain, wherein the antibody domain comprises a functional antibody fragment, a single chain variable fragment (scFv), an Fab, a single-domain antibody (sdAb), a nanobody, a V_{H} domain, a V_{L} domain, a VNAR domain, a V_{HH} domain, a bispecific antibody, a diabody, or a functional fragment or a combination thereof. In some embodiments, the extracellular antigen binding domain comprises a ligand, an extracellular domain of a receptor or an adaptor. In some embodiments, the extracellular antigen binding domain comprises a single extracellular antigen binding domain that is specific for a single antigen. In some embodiments, the extracellular antigen binding domain comprises at least two extracellular antigen binding domains, wherein each of the at least two extracellular antigen binding domains is specific for a different antigen.

In some embodiments, the antigen is a cancer associated antigen, a lineage associated antigen, a pathogenic antigen or an autoimmune antigen. In some embodiments, the antigen comprises a viral antigen. In some embodiments, the antigen is a T lymphocyte antigen. In some embodiments, the antigen is an extracellular antigen. In some embodiments, the antigen is an intracellular antigen. In some embodiments, the antigen is selected from the group consisting of an antigen from Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor VIII (EGFRvIII), Mesothelin, Human Epidermal Growth Factor Receptor 2 (HER2), EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFRβ, SSEA-4, EGFR, NCAM, prostase, PAP, ELF2M, GM3, TEM7R, CLDN6, TSHR, GPRC5D, ALK, Dsg1, Dsg3, IGLL1 and combinations thereof. In some embodiments, the antigen is an antigen of a protein selected from the group consisting of CD2, CD3, CD4, CD5, CD7, CCR4, CD8, CD30, CD45, and CD56. In some embodiments, the antigen is an ovarian cancer antigen or a T lymphoma antigen. In some embodiments, the antigen is an antigen of an integrin receptor. In some embodiments, the antigen is an antigen of an integrin receptor or integrin selected from the group consisting of α1, α2, αIIb, α3, α4, α5, α6, α7, α8, α9, α10, α11, αD, αE, αL, αM, αV, αX, β1, β2, β3, β4, β5, β6, β7, and β8. In some embodiment, the antigen is an antigen of an integrin receptor ligand. In some embodiments, the antigen is an antigen of fibronectin, vitronectin, collagen, or laminin. In some embodiments, the antigen binding domain can bind to two or more different antigens.

In some embodiments, the antigen binding domain comprises an autoantigen or fragment thereof, such as Dsg1 or Dsg3. In some embodiments, the extracellular antigen binding domain comprises a receptor domain or an antibody domain wherein the antibody domain binds to an auto antigen, such as Dsg1 or Dsg3.

In some embodiments, the transmembrane domain and the extracellular antigen binding domain are operatively linked through a linker. In some embodiments, the transmembrane domain and the extracellular antigen binding domain are operatively linked through a linker such as a hinge region of CD8α, IgG1 or IgG4.

In some embodiments, the extracellular domain comprises a multimerization scaffold.

In some embodiments, the transmembrane domain comprises a CD8 transmembrane domain. In some embodiments, the transmembrane domain comprises a CD28 transmembrane domain. In some embodiments, the transmembrane domain comprises a CD68 transmembrane domain. In some embodiments, the transmembrane domain comprises a CD2 transmembrane domain. In some embodiments, the transmembrane domain comprises an FcR transmembrane domain. In some embodiments, the transmembrane domain comprises an FcRγ transmembrane domain. In some embodiments, the transmembrane domain comprises an FcRα transmembrane domain. In some embodiments, the transmembrane domain comprises an FcRβ transmembrane domain. In some embodiments, the transmembrane domain comprises an FcRε transmembrane domain.

Contemplated herein are methods for myeloid specific expression that can be utilized irrespective of whether the nucleic acid is delivered as mRNA or DNA. Accordingly, in one aspect, the CFP is designed such that it leverages the presence of a myeloid endogenous protein for its own expression and functionality. In one aspect, the myeloid specific expression of the CFP is achieved through a design in which the CFP is expressed in the membrane only when it dimerizes (or multimerizes) with one monocyte specific protein. In some embodiments, the myeloid specific functionality of the CFP is achieved through a design in which the CFP is expressed on the membrane only when it dimerizes (or multimerizes) with a monocyte specific protein. In some embodiments, the transmembrane domain comprises a multimerizing domain, such that the transmembrane domain specifically multimerizes with a cell in which the expression of the chimeric receptor is desired to be expressed, for example—a myeloid cell. For example, the transmembrane domain multimerizes with an endogenous transmembrane protein of a myeloid cell, and the multimerization facilitates expression and/or stabilization of the chimeric receptor. FcR alpha receptors oligomerize with FcR gamma receptors for membrane expression on myeloid cells and/or function. Several Fc receptors (FcRs) are endogenously expressed in monocytes and myeloid cells. Exemplary FcR domain sequences used in constructing chimeric fusion protein receptors disclosed herein are provided below:

An exemplary Human CD16 TM sequence incorporated in a CFP (includes an extracellular hinge domain) GLAVSTISSFFPPGYQVSFCLVMVLLFAVDTGLYFSV (SEQ ID NO: 28); wherein the extracellular hinge domain is GLAVSTISSFFPPGYQV (SEQ ID NO: 29) and the TM domain is SFCLVMVLLFAVDTGLYFSV (SEQ ID NO: 30). An exemplary Human CD16 Cytoplasmic domain comprises the sequence KTNIRSSTRDWKDHKFKWRKDPQDK (SEQ ID NO: 31). An exemplary Human CD89 TM sequence incorporated in a CFP (includes an extracellular hinge domain) comprises a sequence IHQDYTTQNLIRMAVAGLVLVALLAILV (SEO ID NO: 32) wherein the Extracellular hinge domain is IHQDYTTQN (SEO ID NO: 33) and the TM domain is LIRMAVAGLVLVALLAILV (SEQ ID NO: 34). An exemplary Human CD89 cytoplasmic domain comprises a sequence ENWHSHTALNKEASADVAEPSWSQQMCQPGLTFARTPSVCK (SEO ID NO: 35).

In some embodiments, the CFP is designed to comprise a transmembrane domain derived from the transmembrane domain of protein selected from Fc gamma RI (CD64). In some embodiments, the CFP is designed to comprise a transmembrane domain derived from the transmembrane domain of protein selected from Fc gamma RIIIA (CD16). In some embodiments, the CFP is designed to comprise a transmembrane domain derived from the transmembrane domain of protein selected from Fc□RIIA (CD32a). In some embodiments, the CFP is designed to comprise a transmembrane domain derived from the transmembrane domain of protein selected from Fc alpha R1 (CD89). Any of the domains in the CFP described herein can be from a suitable mammalian origin, either depending on or irrespective of the origin of any other adjoining domains. Non-human mammalian transmembrane domains are contemplated within the scope of the instant disclosure. In some embodiments, the CFP comprises an intracellular domain from the protein FcγR1 (CD64), FcγRIIIA (CD16), or FcγRIIA (CD32a) from which the TMD is derived. In some embodiments, the TMD has a sequence that is identical to the sequence of the TMD of the protein FcγR1 (CD64), FcγRIIIA (CD16), or FcγRIIA (CD32a).

In some embodiments, the transmembrane domain comprises a transmembrane domain from a syntaxin, such as syntaxin 3 or syntaxin 4 or syntaxin 5. In some embodiments, the transmembrane domain oligomerizes with a transmembrane domain of an endogenous receptor when the CFP is expressed in a cell. In some embodiments, the transmembrane domain oligomerizes with a transmembrane domain of an exogenous receptor when the CFP is expressed in a cell. In some embodiments, the transmembrane domain dimerizes with a transmembrane domain of an endogenous receptor when the CFP is expressed in a cell. In some embodiments, the transmembrane domain dimerizes with a transmembrane domain of an exogenous receptor when the CFP is expressed in a cell. In some embodiments, the transmembrane domain is derived from a protein that is different than the protein from which the intracellular signaling domain is derived. In some embodiments, the transmembrane domain is derived from a protein that is different than the protein from which the extracellular domain is derived. In some embodiments, the transmembrane domain comprises a transmembrane domain of a phagocytic receptor. In some embodiments, the transmembrane domain and the extracellular domain are derived from the same protein. In some embodiments, the transmembrane domain is derived from the same protein as the intracellular signaling domain. In some embodiments, the nucleic acid encodes a DAP12 recruitment domain. In some embodiments, the transmembrane domain comprises a transmembrane domain that oligomerizes with DAP12.

In some embodiments, the transmembrane domain is at least 12, 13, 14, 15, 16, 17, 18, 19, 20, 21, 22, 23, 24, 25, 26, 27, 28, 29, 30, 31 or 32 amino acids in length. In some embodiments, the transmembrane domain is at most 12, 13, 14, 15, 16, 17, 18, 19, 20, 21, 22, 23, 24, 25, 26, 27, 28, 29, 30, 31 or 32 amino acids in length.

In some embodiments, the nucleic acid comprises a sequence encoding a pro-inflammatory polypeptide. In some embodiments, the composition further comprises a proinflammatory nucleotide or a nucleotide in the nucleic acid, for example, an ATP, ADP, UTP, UDP, and/or UDP-glucose.

In some embodiments, the composition further comprises a pro-inflammatory polypeptide. In some embodiments, the pro-inflammatory polypeptide is a chemokine, cytokine. In some embodiments, the chemokine is selected from the group consisting of IL-1, IL3, IL5, IL-6, il8, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon. In some embodiments, the cytokine is selected from the group consisting of IL-1, IL3, IL5, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon.

In some embodiments, the myeloid cells are specifically targeted for delivery. Myeloid cells can be targeted using specialized biodegradable polymers, such as PLGA (poly(lactic-co-glycolic) acid and/or polyvinyl alcohol (PVA). In some embodiments, one or more compounds can be selectively incorporated in such polymeric structures to affect the myeloid cell function. In some embodiments, the targeting structures are multilayered, e.g., of one or more PLGA and one or more PVA layers. In some embodiments, the targeting structures are assembled in an order for a layered activity. In some embodiments, the targeted polymeric structures are organized in specific shaped components, such as labile structures that can adhere to a myeloid cell surface and deliver one or more components such as growth factors and cytokines, such as to maintain the myeloid cell in a microenvironment that endows a specific polarization. In some embodiments, the polymeric structures are such that they are not phagocytosed by the myeloid cell, but they can remain adhered on the surface. In some embodiments the one or more growth factors may be M1 polarization factors, such as a cytokine. In some embodiments the one or more growth factors may be an M2 polarization factor, such as a cytokine. In some embodiments, the one or more growth factors may be a macrophage activating cytokine, such as IFNγ. In some embodiments the polymeric structures are capable of sustained release of the one or more growth factors in an in vivo environment, such as in a solid tumor.

In some embodiments, the nucleic acid comprises a sequence encoding a homeostatic regulator of inflammation. In some embodiments, the homeostatic regulator of inflammation is a sequence in an untranslated region (UTR) of an mRNA. In some embodiments, the sequence in the UTR is a sequence that binds to an RNA binding protein. In some embodiments, translation is inhibited or prevented upon binding of the RNA binding protein to the sequence in an untranslated region (UTR). In some embodiments, the nucleic acid is expressed on a bicistronic vector.

### Engineered Myeloid Cells “Targeted” to Attack Diseased Cells

The present disclosure involves compositions and methods for preparing targeted killer myeloid cells; by leveraging the innate functional role in immune defense, ranging from properties related to detecting foreign bodies, particles, diseased cells, cellular debris, inflammatory signal, chemoattract; activating endogenous DAMP and PAMP signaling pathways; trigger myelopoiesis, extravasation; chemotaxis; phagocytes; pinocytosis; recruitment; engulfment; scavenging; activating intracellular oxidative burst and lysis or killing of pathogens, detecting, engulfing and killing diseased or damaged cells; removing unwanted cellular, tissue or acellular debris in vivo; antigen presentation and role in activating innate immunity; activating and modulating an immune response cascade; activating T cell repertoire; autophagy; inflammatory and non-inflammatory apoptosis; pyroptosis, immune editing to response to stress and restoration of tissue homeostasis. In one aspect, provided herein are methods and compositions to augment one or more functions of a myeloid cell for use in a therapeutic application, the one or more functions may be one or more of: detecting foreign bodies, particles, diseased cells, cellular debris, inflammatory signal, chemoattract; activating endogenous DAMP and PAMP signaling pathways; trigger myelopoiesis, extravasation; chemotaxis; phagocytosis; pinocytosis; recruitment; trogocytosis; engulfment; scavenging; activating intracellular oxidative burst and intracellular lysis or killing of pathogens, detecting, engulfing and killing diseased or damaged cells; removing unwanted cellular, tissue or acellular debris in vivo; antigen presentation and role in activating innate immunity; activating and modulating an immune response cascade; activating T cell repertoire; autophagy; inflammatory and non-inflammatory apoptosis; pyroptosis, immune editing to response to stress and restoration of tissue homeostasis. In one embodiment, the compositions and methods are also directed to augmenting the targeting, and killing function of certain myeloid cells, by genetic modification of these cells. The compositions and methods described herein are directed to creating engineered myeloid cells, wherein the engineered myeloid cells comprise at least one genetic modification, and can be directed to recognize and induce effector functions against a pathogen, a diseased cell, such as a tumor or cancer cell, such that the engineered myeloid cell is capable of recognizing, targeting, phagocytosing, killing and/or eliminating the pathogen or the diseased cell or the cancer cell, and additionally, may activate a specific immune response cascade following the phagocytosis, killing and/or eliminating the pathogen or the diseased cell.

Myeloid cells appear to be the most abundant cells in a tumor. Myeloid cells are also capable of recognizing a tumor cell over a healthy normal cell of the body and mount an immune response to a tumor cell of the body. As sentinels of innate immune response, myeloid cells can sense non-self or aberrant cell types and clear them via a process called phagocytosis. This can be directed to a therapeutic advantage in driving myeloid cell mediated phagocytosis and lysis of tumor cells. However, these naturally occurring tumor-infiltrating myeloid cells (TIMs) may be subjected to influence of the tumor microenvironment (TME). TIMs constitute a heterogeneous population of cells. Many TIMs originate from circulating monocytes and granulocytes, which in turn stem from bone marrow-derived hematopoietic stem cells. However, in the presence of persistent stimulation by tumor-derived factors the monocyte and granulocyte progenitors divert from their intrinsic pathway of terminal differentiation into mature macrophages, DCs or granulocytes, and may become tumor promoting myeloid cell types. Differentiation into pathological, alternatively activated immature myeloid cells is favored. These immature myeloid cells include tumor-associated DCs (TADCs), tumor-associated neutrophils (TANs), myeloid-derived suppressor cells (MDSCs), and tumor-associated macrophages (TAMs). Alternative to this emergency myelopoiesis, TAMs may also originate from tissue-resident macrophages, which in turn can be of embryonic or monocytic origin. These tissue-resident macrophages undergo changes in phenotype and function during carcinogenesis, and proliferation may help to maintain TAMs derived from tissue-resident macrophages. A tumor microenvironment may drive a tumor infiltrating myeloid cell to become myeloid derived suppressor cells and acquire the ability to suppress T cells. As a result, innovative methods are necessary to create therapeutically effective TIMs that can infiltrate a tumor and can target tumor cells for phagocytic uptake and killing.

In one aspect, provided herein are engineered myeloid cells that can target specific target cells, for example, tumor cells or pathogenic cells. In some embodiments, engineered myeloid cells provided herein are potent in infiltrating, targeting, and killing tumor cells. An engineered myeloid/phagocytic cell described herein is designed to comprise a nucleic acid, which encodes one or more proteins that help target the phagocytic cell to a target cell, for example a tumor cell or a cancer cell. In one embodiment, the engineered myeloid cell is capable of readily infiltrating a tumor. In one embodiment, the engineered myeloid cell has high specificity for the target cell, with none or negligible cross-reactivity to a non-tumor, non-diseased cell of the subject while in circulation. In one embodiment, the engineered myeloid/phagocytic cell described herein is designed to comprise a nucleic acid, which will help the cell to overcome/bypass the TME influence and mount a potent anti-tumor response. In one embodiment, the engineered myeloid/phagocytic cell described herein is designed to comprise a nucleic acid, which augments phagocytosis of the target cell. In another embodiment, the engineered myeloid/phagocytic cell described herein is designed to comprise a nucleic acid, which augments reduce or eliminate trogocytosis and/or enhance phagocytic lysis or of the target cell.

Accordingly, in some embodiments, the compositions disclosed herein comprise a myeloid cell, comprising a nucleic acid encoding a chimeric receptor fusion protein (CFP), for example, a phagocytic receptor (PR) fusion protein (PFP). The nucleic acid can comprise a sequence encoding a PR subunit comprising: (i) a transmembrane domain, and (ii) an intracellular domain comprising a PR intracellular signaling domain; and an extracellular antigen binding domain specific to an antigen of a target cell; wherein the transmembrane domain and the extracellular antigen binding domain are operatively linked; wherein the PR intracellular signaling domain is derived from a receptor with a signal transduction domain. The nucleic acid further encodes for one or more polypeptides that constitute one or more plasma membrane receptors that helps engage the phagocytic cell to the target cell and enhance its phagocytic activity.

In some embodiments, the myeloid cell described herein comprises one or more recombinant proteins comprising a chimeric receptor, wherein the chimeric receptor can respond to a first phagocytic signal directed to a target cell, which may be a diseased cell, a tumor cell or a pathogen, and a second signal, which is an inflammatory signal, that augments the phagocytic and killing response to target initiated by the first signal. In some embodiments, the recombinant proteins may comprise one or more of the amino acid sequences depicted in Table 1A.

TABLE 1A
Sequences of chimeric fusion proteins and domains thereof
(Respective CDR1, 2 and 3 sequences underlined, Kabat nomenclature)

| Protein / domain | Sequence |
|---|---|
| Anti-CD5 heavy chain | EIQLVQSGGGLVKPGGSVRISCAASGYTFTNYGMNWVRQAPGK |
| variable domain | GLEWMGWINTHTGEPTYADSFKGRFTFSLDDSKNTAYLQINSLR |
| | AEDTAVYFCTRRGYDWYFDVWGQGTTVTV (SEQ ID NO: 36) |
| Anti-CD5 light chain | DIQMTQSPSSLSASVGDRVTITCRASQDINSYLSWFQQKPGKAPK |
| variable domain | TLIYRANRLESGVPSRFSGSGSGTDYTLTISSLQYEDFGIYYCQQY |
| | DESPWTFGGGTKLEIK (SEQ ID NO: 37) |
| Anti-CD5 scFv | EIQLVQSGGGLVKPGGSVRISCAASGYTFTNYGMNWVRQAPGK |
| | GLEWMGWINTHTGEPTYADSFKGRFTFSLDDSKNTAYLQINSLR |
| | AEDTAVYFCTRRGYDWYFDVWGQGTTVTVSSGGGGSGGGGSG |
| | GGGSDIQMTQSPSSLSASVGDRVTITCRASQDINSYLSWFQQKPG |
| | KAPKTLIYRANRLESGVPSRFSGSGSGTDYTLTISSLQYEDFGIYY |
| | CQQYDESPWTFGGGTKLEIK (SEQ ID NO: 38) |
| FcRγ-chain | LYCRRLKIQVRKAAITSYEKSDGVYTGLSTRNQETYETLKHEKPP |
| intracellular signaling | Q (SEQ ID NO: 39) |
| domain | |
| FcRγ-chain | LYCRLKIQVRKAAITSYEKSDGVYTGLSTRNQETYETLKHEKPP |
| intracellular signaling | Q (SEQ ID NO: 40) |
| domain | |
| FcRγ-chain | RLKIQVRKAAITSYEKSDGVYTGLSTRNQETYETLKHEKPPQ |
| intracellular signaling | (SEQ ID NO: 41) |
| domain | |
| FcRγ-chain | RLKIQVRKAAITSYEKSDGVYTGLSTRNQETYETLKHEKPPQ |
| intracellular signaling | (SEQ ID NO: 41) |
| domain | |
| PI3K recruitment | YEDMRGILYAAPQLRSIRGQPGPNHEEDADSYENM (SEQ ID NO: |
| domain | 42) |
| CD40 intracellular | KKVAKKPTNKAPHPKQEPQEINFPDDLPGSNTAAPVQETLHGCQ |
| domain | PVTQEDGKESRISVQERQ (SEQ ID NO: 43) |
| CD8α chain | IYIWAPLAGTCGVLLLSLVIT (SEQ ID NO: 44) |
| transmembrane | |
| domain | |
| CD8α chain | IYIWAPLAGTCGVLLLSLVITLYC (SEQ ID NO: 45) |
| transmembrane | |
| domain | |
| CD8α chain hinge | ALSNSIMYFSHFVPVFLPAKPTTTPAPRPPTPAPTIASQPLSLRPEA |
| domain | CRPAAGGAVHTRGLD (SEQ ID NO: 46) |
| Anti-HER2 heavy | DIQMTQSPSSLSASVGDRVTITCRASQDVNTAVAWYQQKPGKAP |
| chain variable domain | KLLIYSASFLYSGVPSRFSGSRSGTDFTLTISSLQPEDFATYYCQQ |
| | HYTTPPTFGQGTKVEIKRTGSTSGSGKPGSGEGSEVQLVE (SEQ |
| | ID NO: 47) |
| Anti-HER2 light chain | LVQPGGSLRLSCAASGFNIKDTYIHWVRQAPGKGLEWVARIYPT |
| variable domain | NGYTRYADSVKGRFTISADTSKNTAYLQMNSLRAEDTAVYYCS |
| | RWGGDGFYAMDVWGQGTLVTV (SEQ ID NO: 48) |
| Anti-HER2 scFv | DIQMTQSPSSLSASVGDRVTITCRASQDVNTAVAWYQQKPGKAP |
| | KLLIYSASFLYSGVPSRFSGSRSGTDFTLTISSLQPEDFATYYCQQ |
| | HYTTPPTFGQGTKVEIKRTGSTSGSGKPGSGEGSEVQLVESSGGG |
| | GSGGGGSGGGGSLVQPGGSLRLSCAASGFNIKDTYIHWVRQAPG |
| | KGLEWVARIYPTNGYTRYADSVKGRFTISADTSKNTAYLQMNS |
| | LRAEDTAVYYCSRWGGDGFYAMDVWGQGTLVTV (SEQ ID NO: |
| | 49) |
| GMCSF Signal | MWLQSLLLLGTVACSIS (SEQ ID NO: 50) |
| peptide | |
| CD28 transmembrane | FWVLVVVGGVLACYSLLVTVAFIIFWV (SEQ ID NO: 51) |
| domain | |
| CD2 Transmembrane | IYLIIGICGGGSLLMVFVALLVFYIT (SEQ ID NO: 52) |
| domain | |
| CD68 transmembrane | ILLPLIIGLILLGLLALVLIAFCII (SEQ ID NO: 53) |
| domain | |
| TNFR1 intracellular | QRWKSKLYSIVCGKSTPEKEGELEGTTTKPLAPNPSFSPTPGFTPT |
| domain | LGFSPVPSSTFTSSSTYTPGDCPNFAAPRREVAPPYQGADPILATA |
| | LASDPIPNPLQKWEDSAHKPQSLDTDDPATLYAVVENVPPLRWK |
| | EFVRRLGLSDHEIDRLELQNGRCLREAQYSMLATWRRRTPRREA |
| | TLELLGRVLRDMDLLGCLEDIEEALCGPAALPPAPSLLR (SEQ ID |
| | NO: 54) |
| TNFR2 intracellular | PLCLQREAKVPHLPADKARGTQGPEQQHLLITAPSSSSSSLESSAS |
| domain | ALDRRAPTRNQPQAPGVEASGAGEARASTGSSDSSPGGHGTQV |
| | NVTCIVNVCSSSDHSSQCSSQASSTMGDTDSSPSESPKDEQVPFS |
| | KEECAFRSQLETPETLLGSTEEKPLPLGVPDAGMKPS (SEQ ID |
| | NO: 55) |
| MDA5 intracellular | MSNGYSTDENFRYLISCFRARVKMYIQVEPVLDYLTFLPAEVKE |
| domain | QIQRTVATSGNMQAVELLLSTLEKGVWHLGWTREFVEALRRTG |
| | SPLAARYMNPELTDLPSPSFENAHDEYLQLLNLLQPTLVDKLLV |
| | RDVLDKCMEEELLTIEDRNRIAAAENNGNESGVRELLKRIVQKE |
| | NWFSAFLNVLRQTGNNELVQELTGSDCSESNAEIEN (SEQ ID |
| | NO: 56) |
| CD8α chain hinge | ALSNSIMYFSHFVPVFLPAKPTTTPAPRPPTPAPTIASQPLSLRPEA |
| domain + | CRPAAGGAVHTRGLDIYIWAPLAGTCGVLLLSLVITLYC (SEQ ID |
| transmembrane | NO: 57) |
| domain | |
| CD8α chain hinge | ALSNSIMYFSHFVPVFLPAKPTTTPAPRPPTPAPTIASQPLSLRPEA |
| domain + | CRPAAGGAVHTRGLDIYIWAPLAGTCGVLLLSLVIT (SEQ ID NO: |
| transmembrane | 58) |
| domain | |
| CD5-FcRγ-PI3K | MWLQSLLLLGTVACSISEIQLVQSGGGLVKPGGSVRISCAASGYT |
| | FTNYGMNWVRQAPGKGLEWMGWINTHTGEPTYADSFKGRFTF |
| | SLDDSKNTAYLQINSLRAEDTAVYFCTRRGYDWYFDVWGQGTT |
| | VTVSSGGGGSGGGGSGGGGSDIQMTQSPSSLSASVGDRVTITCRA |
| | SQDINSYLSWFQQKPGKAPKTLIYRANRLESGVPSRFSGSGSGTD |
| | YTLTISSLQYEDFGIYYCQQYDESPWTFGGGTKLEIKSGGGGSGA |
| | LSNSIMYFSHFVPVFLPAKPTTTPAPRPPTPAPTIASQPLSLRPEAC |
| | RPAAGGAVHTRGLDIYIWAPLAGTCGVLLLSLVITLYCRRLKIQV |
| | RKAAITSYEKSDGVYTGLSTRNQETYETLKHEKPPQGSGSYEDM |
| | RGILYAAPQLRSIRGQPGPNHEEDADSYENM (SEQ ID NO: 59) |
| HER2-FcRγ-PI3K | MWLQSLLLLGTVACSISDIQMTQSPSSLSASVGDRVTITCRASQD |
| | VNTAVAWYQQKPGKAPKLLIYSASFLYSGVPSRFSGSRSGTDFT |
| | LTISSLQPEDFATYYCQQHYTTPPTFGQGTKVEIKRTGSTSGSGKP |
| | GSGEGSEVQLVESGGGLVQPGGSLRLSCAASGFNIKDTYIHWVR |
| | QAPGKGLEWVARIYPTNGYTRYADSVKGRFTISADTSKNTAYLQ |
| | MNSLRAEDTAVYYCSRWGGDGFYAMDVWGQGTLVTVSSSGGG |
| | GSGALSNSIMYFSHFVPVFLPAKPTTTPAPRPPTPAPTIASQPLSLR |
| | PEACRPAAGGAVHTRGLDIYIWAPLAGTCGVLLLSLVITLYCRRL |
| | KIQVRKAAITSYEKSDGVYTGLSTRNQETYETLKHEKPPQGSGSY |
| | EDMRGILYAAPQLRSIRGQPGPNHEEDADSYENM (SEQ ID NO: |
| | 60) |
| CD5-FcRγ-CD40 | MWLQSLLLLGTVACSISEIQLVQSGGGLVKPGGSVRISCAASGYT |
| | FTNYGMNWVRQAPGKGLEWMGWINTHTGEPTYADSFKGRFTF |
| | SLDDSKNTAYLQINSLRAEDTAVYFCTRRGYDWYFDVWGQGTT |
| | VTVSSGGGGSGGGGSGGGGSDIQMTQSPSSLSASVGDRVTITCRA |
| | SQDINSYLSWFQQKPGKAPKTLIYRANRLESGVPSRFSGSGSGTD |
| | YTLTISSLQYEDFGIYYCQQYDESPWTFGGGTKLEIKSGGGGSGA |
| | LSNSIMYFSHFVPVFLPAKPTTTPAPRPPTPAPTIASQPLSLRPEAC |
| | RPAAGGAVHTRGLDIYIWAPLAGTCGVLLLSLVITLYCRLKIQVR |
| | KAAITSYEKSDGVYTGLSTRNQETYETLKHEKPPQKKVAKKPTN |
| | KAPHPKQEPQEINFPDDLPGSNTAAPVQETLHGCQPVTQEDGKE |
| | SRISVQERQ (SEQ ID NO: 61) |
| CD5-FcRγ-MDA5 | MWLQSLLLLGTVACSISEIQLVQSGGGLVKPGGSVRISCAASGYT |
| | FTNYGMNWVRQAPGKGLEWMGWINTHTGEPTYADSFKGRFTF |
| | SLDDSKNTAYLQINSLRAEDTAVYFCTRRGYDWYFDVWGQGTT |
| | VTVSSGGGGSGGGGSGGGGSDIQMTQSPSSLSASVGDRVTITCR |
| | ASQDINSYLSWFQQKPGKAPKTLIYRANRLESGVPSRFSGSGSGT |
| | DYTLTISSLQYEDFGIYYCQQYDESPWTFGGGTKLEIKSGGGGSG |
| | ALSNSIMYFSHFVPVFLPAKPTTTPAPRPPTPAPTIASQPLSLRPEA |
| | CRPAAGGAVHTRGLDIYIWAPLAGTCGVLLLSLVITLYCRLKIQV |
| | RKAAITSYEKSDGVYTGLSTRNQETYETLKHEKPPQGSGSMSNG |
| | YSTDENFRYLISCFRARVKMYIQVEPVLDYLTFLPAEVKEQIQRT |
| | VATSGNMQAVELLLSTLEKGVWHLGWTREFVEALRRTGSPLAA |
| | RYMNPELTDLPSPSFENAHDEYLQLLNLLQPTLVDKLLVRDVLD |
| | KCMEEELLTIEDRNRIAAAENNGNESGVRELLKRIVQKENWFSA |
| | FLNVLRQTGNNELVQELTGSDCSESNAEIEN (SEQ ID NO: 62) |
| CD5-FcRγ-TNFR1 | MWLQSLLLLGTVACSISEIQLVQSGGGLVKPGGSVRISCAASGYT |
| | FTNYGMNWVRQAPGKGLEWMGWINTHTGEPTYADSFKGRFTF |
| | SLDDSKNTAYLQINSLRAEDTAVYFCTRRGYDWYFDVWGQGTT |
| | VTVSSGGGGSGGGGSGGGGSDIQMTQSPSSLSASVGDRVTITCR |
| | ASQDINSYLSWFQQKPGKAPKTLIYRANRLESGVPSRFSGSGSGT |
| | DYTLTISSLQYEDFGIYYCQQYDESPWTFGGGTKLEIKSGGGGSG |
| | ALSNSIMYFSHFVPVFLPAKPTTTPAPRPPTPAPTIASQPLSLRPEA |
| | CRPAAGGAVHTRGLDIYIWAPLAGTCGVLLLSLVITLYCRLKIQV |
| | RKAAITSYEKSDGVYTGLSTRNQETYETLKHEKPPQGSGSQRWK |
| | SKLYSIVCGKSTPEKEGELEGTTTKPLAPNPSFSPTPGFTPTLGFSP |
| | VPSSTFTSSSTYTPGDCPNFAAPRREVAPPYQGADPILATALASDP |
| | IPNPLQKWEDSAHKPQSLDTDDPATLYAVVENVPPLRWKEFVRR |
| | LGLSDHEIDRLELQNGRCLREAQYSMLATWRRRTPRREATLELL |
| | GRVLRDMDLLGCLEDIEEALCGPAALPPAPSLLR (SEQ ID NO: |
| | 63) |
| CD5-FcRγ-TNFR2 | MWLQSLLLLGTVACSISEIQLVQSGGGLVKPGGSVRISCAASGYT |
| | FTNYGMNWVRQAPGKGLEWMGWINTHTGEPTYADSFKGRFTF |
| | SLDDSKNTAYLQINSLRAEDTAVYFCTRRGYDWYFDVWGQGTT |
| | VTVSSGGGGSGGGGSGGGGSDIQMTQSPSSLSASVGDRVTITCR |
| | ASQDINSYLSWFQQKPGKAPKTLIYRANRLESGVPSRFSGSGSGT |
| | DYTLTISSLQYEDFGIYYCQQYDESPWTFGGGTKLEIKSGGGGSG |
| | ALSNSIMYFSHFVPVFLPAKPTTTPAPRPPTPAPTIASQPLSLRPEA |
| | CRPAAGGAVHTRGLDIYIWAPLAGTCGVLLLSLVITLYCRLKIQV |
| | RKAAITSYEKSDGVYTGLSTRNQETYETLKHEKPPQGSGSPLCLQ |
| | REAKVPHLPADKARGTQGPEQQHLLITAPSSSSSSLESSASALDR |
| | RAPTRNQPQAPGVEASGAGEARASTGSSDSSPGGHGTQVNVTCI |
| | VNVCSSSDHSSQCSSQASSTMGDTDSSPSESPKDEQVPFSKEECA |
| | FRSQLETPETLLGSTEEKPLPLGVPDAGMKPS (SEQ ID NO: 64) |

TABLE 1B
Linker sequences
Sequence

SSGGGGSGGGGSGGGGS (SEQ ID NO: 65)
SGGGGSG (SEQ ID NO: 66)
SGGG (SEQ ID NO: 67)
GSGS (SEQ ID NO: 68)

In some embodiments, the therapeutically effective myeloid cell comprises or presents or expresses one or more an exogenous or recombinant tumor antigens. In some embodiments, the tumor antigens are tissue specific antigens. In some embodiments, the tumor antigens are endogenous overexpressed antigens. In some embodiments, the tumor antigens are mutated protein antigens. In some embodiments, the therapeutically effective myeloid cell comprises or presents or expresses a melanoma antigen such as a Tyrosinase-related Protein 2 (TRP2) antigen, such as a TRP2 epitope, such as amino acids 180-188 of TRP2 (SVYDFFVWL (SEQ ID NO: 69)). In some embodiments, the therapeutically effective myeloid cell comprises or presents or expresses a mutant antigen, for example a glioblastoma antigen, e.g., an isocitrate dehydrogenase 1 (mIDHI) antigen, such as a mutant IDH1 antigen (R132H), such as GWVKPIIIGHHAYGDQYRATDFVVP (SEQ ID NO: 70). In some embodiments, a method can comprise administering a myeloid cell that comprises or presents or expresses one or more an exogenous or recombinant antigens to treat a cancer, such as a brain cancer, a glioma or a glioblastoma.

mRNA Compositions Comprising Modifications for Prolonged Expression of Encoded Proteins in Myeloid Cells

Provided herein are modifications in an engineered polynucleic acid comprising a sequence encoding a protein or a peptide, designed for specific and enhanced expression of the protein or peptide encoded therein. In some embodiments, an engineered polynucleic acid is described herein, e.g., a recombinant mRNA, (in some embodiment interchangeably referred to a mRNA construct), comprising a 5′ untranslated region (UTR), a coding sequence downstream of the 5′ UTR encoding, for example, a therapeutic protein and followed by a 3′UTR downstream of the coding sequence, wherein the 5′UTR and the 3′UTR are heterologous to the coding sequence and designed for enhancing the expression of the sequence encoding the therapeutic protein.

In some embodiments, the 5′UTR and the 3′UTR in recombinant or engineered polynucleic acid (mRNA) are from heterologous sources. In some embodiments, the 5′UTR and the 3′UTR in recombinant or engineered polynucleic acid (mRNA) are from the same gene.

In some embodiments, the 5′ UTR comprises a sequence that has at least 80% identity to the 5′UTR of bovine growth hormone gene (BGH). In some embodiments, the 5′ UTR comprises a sequence that has at least 81%, 82%, 83%, 84%, 85%, 86%, 87%, 88%, 89%, 90%, 91%, 92%, 93%, 94%, 95%, 96%, 97%, 98%, or at least 99% sequence identity to the 5′UTR of BGH.

In some embodiments, the 3′ UTR comprises a sequence that has at least 80% identity to the 3′UTR of bovine growth hormone gene (BGH). In some embodiments, the 3′ UTR comprises a sequence that has at least 81%, 82%, 83%, 84%, 85%, 86%, 87%, 88%, 89%, 90%, 91%, 92%, 93%, 94%, 95%, 96%, 97%, 98%, or at least 99% sequence identity to the 3′UTR of BGH.

In some embodiments, a 5′UTR may be derived from BGH, and may comprise a sequence that is extensively modified. In some embodiments, a 3′UTR may be derived from BGH, and may comprise a sequence that is extensively modified.

In some embodiments, the 5′ UTR comprises a sequence that has at least 80% identity to the 5′UTR of a globin gene. In some embodiments, the 5′ UTR comprises a sequence that has at least 80% identity to the 5′UTR of human globin gene. In some embodiments, the 5′ UTR comprises a sequence that has at least 81%, 82%, 83%, 84%, 85%, 86%, 87%, 88%, 89%, 90%, 91%, 92%, 93%, 94%, 95%, 96%, 97%, 98%, or at least 99% sequence identity to the 5′UTR of human globin gene.

In some embodiments, the 3′ UTR comprises a sequence that has at least 80% identity to the 3′UTR of a bovine globin gene. In some embodiments, the 3′ UTR comprises a sequence that has at least 80% identity to the 3′UTR of human globin gene. In some embodiments, the 3′ UTR comprises a sequence that has at least 81%, 82%, 83%, 84%, 85%, 86%, 87%, 88%, 89%, 90%, 91%, 92%, 93%, 94%, 95%, 96%, 97%, 98%, or at least 99% sequence identity to the 3′UTR of human globin gene.

In some embodiments, a 5′UTR may be derived from globin gene, and may comprise a sequence that is extensively modified. In some embodiments, a 3′UTR may be derived from globin gene, and may comprise a sequence that is extensively modified. In some embodiment, a 5′ UTR or a 3′ UTR may comprise an artificial sequence. An artificial sequence is a sequence of oligonucleotides or polynucleotides that are not present in nature, but may be synthesized. In some embodiments, a 5′UTR comprises a Kozak sequence. In some embodiments, a 5′UTR comprises a 5′ cap structure. The 5′ cap may be a cap 1 or a cap 0 structure, or may comprise an ARCA cap (ARCA, anti-reverse cap analog). In some embodiments, the 3′ UTR further comprises a poly A tail. In some embodiments, a poly A tail is attached enzymatically to the 3′ UTR of the mRNA. In some embodiments, the polyA tail is incorporated to the mRNA after the mRNA is prepared, for example, via in vitro translation (IVT) and in some embodiments, after a first round of purification of the IVT generated mRNA. In some embodiments, a poly A tail length is regulated. In some embodiments, there are about 80-200 adenosine (A) residues in the poly A tail.

In some embodiments a 5′ UTR may comprise a sequence of 5′-GGGAGACCCAAGCTGGCTAGCGTTTAAACTTAAGCTT-3′ (SEQ ID NO: 71). As is understood throughout this document, any mRNA sequence may be presented herein, where the T (Thymine) residue may be read as U (Uracil) residues, for example, the above sequence is 5′-GGGAGACCCAAGCUGGCUAGCGUUUAAACUUAAGCUU-3′ (SEQ ID NO: 72). In some embodiments, a 5′ UTR may comprise a sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to 5′-GGGAGACCCAAGCUGGCUAGCGUUUAAACUUAAGCUU-3′ (SEQ ID NO: 72).

In some embodiments, the 3′ UTR comprises a sequence:

(SEQ ID NO: 73)
5′-CTCGAGTCTAGAGGGCCCGTTTAAACCCGCTGATCAGCCTCGACTGT
GCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGT
GGGGCAGGACAGCAAGGGGGAGGATTGGGAAGACAATAGC-3′.
(Underlined region corresponds to BGH poly A
signal).

As indicated previously, the 3′UTR comprises a sequence:

(SEQ ID NO: 74)
5′-CUCGAGUCUAGAGGGCCCGUUUAAACCCGCUGAUCAGCCUCGACUGU
GCCUUCUAGUUGCCAGCCAUCUGUUGUUUGCCCCUCCCCCGUGCCUUCCU
UGACCCUGGAAGGUGCCACUCCCACUGUCCUUUCCUAAUAAAAUGAGGAA
AUUGCAUCGCAUUGUCUGAGUAGGUGUCAUUCUAUUCUGGGGGGUGGGGU
GGGGCAGGACAGCAAGGGGGAGGAUUGGGAAGACAAUAGC-3′.

In some embodiments, the 3′ UTR comprises a sequence that has at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to

(SEQ ID NO: 74)
5′-CUCGAGUCUAGAGGGCCCGUUUAAACCCGCUGAUCAGCCUCGACUGU
GCCUUCUAGUUGCCAGCCAUCUGUUGUUUGCCCCUCCCCCGUGCCUUCCU
UGACCCUGGAAGGUGCCACUCCCACUGUCCUUUCCUAAUAAAAUGAGGAA
AUUGCAUCGCAUUGUCUGAGUAGGUGUCAUUCUAUUCUGGGGGGUGGGGU
GGGGCAGGACAGCAAGGGGGAGGAUUGGGAAGACAAUAGC-3′

In some embodiments, mRNA constructs comprising sequences encoding one or more polypeptides such as the CFPs described herein, wherein the mRNA are constructs are designed to include specific 5′- and 3′ UTRs, (per convention, read from 5′ to 3′ direction for each sequence) that provide enhanced intracellular stability and prolonged expression of the encoded polypeptides. Exemplary UTRs may include:

5′ UTR A1:
(SEQ ID NO: 75)
5′-GGGAAAUAAGAGAGAAAAGAAGAGUAAGAAGAAAUAUAAGAGCCAC
C -3′
5′ UTR B1:
(SEQ ID NO: 76)
5′ACUCCUCCCCAUCCUCUCCCUCUGUCCCUCUGUCCCUCUGACCCUGCA
CUGUCCCAGCACC -3′
5′ UTR C1:
(SEQ ID NO: 77)
5′-ACAUUUGCUUCUGACACAACUGUGUUCACUAGCAACCUCAAACAGAC
ACC -3′
5′ UTR D1:
(SEQ ID NO: 78)
5′-CTTGCCCGCCGATATCTCTGCCGGGTGACTAGCTGCTTCCTTTCTCT
CTCGCGCGCGGTGTGGTGGCAGCAGGCGCAGCCCAGCCTCGAA-3′

The above sequence of 5′UTR DI is alternatively depicted below:
(SEQ ID NO: 79)
5′-
CUUGCCCGCCGAUAUCUCUGCCGGGUGACUAGCUGCUUCCUUUCUCUCUCGCGCGCG
GUGUGGUGGCAGCAGGCGCAGCCCAGCCUCGAA-3′
5′ UTR E1:
(SEQ ID NO: 80)
5′-
CTTCCTTTTTGTCCGACATCTTGACGAGGCTGCGGTGTCTGCTGCTATTCTCCGAGCTTC
GCA-3′
The above sequence of 5′UTR El is alternatively depicted below:
′(SEQ ID NO: 81)
5′-
CUUCCUUUUUGUCCGACAUCUUGACGAGGCUGCGGUGUCUGCUGCUAUUCUCCGAGC
UUCGCA-3′
5′ UTR F1:
(SEQ ID NO: 82)
5′-
AGCAATCCTTTCTTTCAGCTGGAGTGCTCCTCAGGAGCCAGCCCCACCCTTAGAAAAG-
3′
The above sequence of 5′ UTR F1 may be alternatively depicted as:
(SEQ ID NO: 83)
5′-AGCAAUCCUUUCUUUCAGCUGGAGUGCUCCUCAGGAGCCAGCCCCACCCUUAGAAAA
G-3′
3′ UTR A2:
(SEQ ID NO: 84)
5′-
UGAUAAUAGGCUGGAGCCUCGGUGGCCAUGCUUCUUGCCCCUUGGGCCUCCCCCCAG
CCCCUCCUCCCCUUCCUGCACCCGUACCCCCGUGGUCUUUGAAUAAAGUCUGA-3′
3′ UTR B2:
(SEQ ID NO: 85)
5′-
CAGGACACAGCCUUGGAUCAGGACAGAGACUUGGGGGCCAUCCUGCCCCUCCAACCC
GACAUGUGUACCUCAGCUUUUUCCCUCACUUGCAUCAAUAAAGCUUCUGUGUUUGGA
ACAG-3′
3′ UTR C2:
(SEQ ID NO: 86)
5′-
GCUCGCUUUCUUGCUGUCCAAUUUCUAUUAAAGGUUCCUUUGUUCCCUAAGUCCAAC
UACUAAACUGGGGGAUAUUAUGAAGGGCCUUGAGCAUCUGGAUUCUGCCUAAUAAA
A-3′
3′ UTR C2-2x:
(SEQ ID NO: 87)
5′-
GCUCGCUUUCUUGCUGUCCAAUUUCUAUUAAAGGUUCCUUUGUUCCCUAAGUCCAAC
UACUAAACUGGGGGAUAUUAUGAAGGGCCUUGAGCAUCUGGAUUCUGCCUAAUAAA
AGCUCGCUUUCUUGCUGUCCAAUUUCUAUUAAAGGUUCCUUUGUUCCCUAAGUCCAA
CUACUAAACUGGGGGAUAUUAUGAAGGGCCUUGAGCAUCUGGAUUCUGCCUAAUAA
AA -3′.
3′ UTR D2:
(SEQ ID NO: 88)
5′- CTGGAGAGAATCACAGATGTGGAATATTTGTCATAAATAAATAATGAAAACCT -3′
3′ UTR E2:
(SEQ ID NO: 89)
5′-ATAGGTCCAACCAGCTGTACATTTGGAAAAATAAAACTTTATTAAATCAAA-3′
3′ UTR F2:
(SEQ ID NO: 90)
5′-
CAAGCACGCAGCAATGCAGCTCAAAACGCTTAGCCTAGCCACACCCCCACGGGAAACA
GCAGTGATTAACCTTTAGCAATAAACGAAAGTTTAACTAAGCTATACTAACCCCAGGGT
TGGTCAATTTCGTGCCAGCCACACCCTGGTACTGCATGCACGCAATGCTAGCTGCCCCT
TTCCCGTCCTGGGTACCCCGAGTCTCCCCCGACCTCGGGTCCCAGGTATGCTCCCACCTC
CACCTGCCCCACTCACCACCTCTGCTAGTTCCAGACACCTCC -3′.
The above sequence of 3′UTR F2 is alternatively depicted below:
(SEQ ID NO: 91)
5′-
CAAGCACGCAGCAAUGCAGCUCAAAACGCUUAGCCUAGCCACACCCCCACGGGAAAC
AGCAGUGAUUAACCUUUAGCAAUAAACGAAAGUUUAACUAAGCUAUACUAACCCCA
GGGUUGGUCAAUUUCGUGCCAGCCACACCCUGGUACUGCAUGCACGCAAUGCUAGCU
GCCCCUUUCCCGUCCUGGGUACCCCGAGUCUCCCCCGACCUCGGGUCCCAGGUAUGCU
CCCACCUCCACCUGCCCCACUCACCACCUCUGCUAGUUCCAGACACCUCC-3′
3′ UTR G2:
(SEQ ID NO: 92)
5′-
GCUGAAGAAGUGGGAAUGGGAGCACUCUGUCUUCUUUGCUAGAGAAGUGGAGAGAA
AAUACAAAAGGUAAAGCAGUUGAGAUUCUCUACAACCUAAAAAAUUCCUAGGUGCU
AUUUUCUUAUCCUUUGUACUGUAGCUAAAUGUACCUGAGACAUAUUAGUCUUUGAA
AAAUAAAGUUAUGUAAGGUUUUUUUUAUCUUUAAAUAGCUCUGUGGGUUUUAACAU
UUUUAUAAAGAUAUACCAA-3′

In some embodiments, an engineered polynucleic acid comprises a 5′ UTR, a coding sequence encoding a therapeutic protein and followed by a 3′UTR, wherein the 5′UTR and the 3′UTR are heterologous to the coding sequence and designed for enhancing the expression of the sequence encoding the therapeutic protein.

The engineered nucleic acid can be generated following molecular biology techniques known to one of skill in the art. The methods include but are not limited to designing primers, generating PCR amplification products, restriction digestion, ligation, cloning, gel purification of cloned product, bacterial propagation of cloned DNA, isolation and purification of cloned plasmid or vector. General guidance can be found in: Molecular Cloning of PCR Products: by Michael Finney, Paul E. Nisson, Ayoub Rashtchian in Current Protocols in Molecular Biology, Volume 56, Issue 1 (First published: 1 Nov. 2001); Recombinational Cloning by Jaehong Park, Joshua LaBaer in Current Protocols in Molecular Biology Volume 74, Issue 1 (First published: 15 May 2006) and others. In some embodiments specific amplification techniques may be used, such as TAS technique (Transcription-based Amplification System), described by Kwoh et al. in 1989; the 3SR technique, which are hereby incorporated by reference. (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

The engineered nucleic acid may comprise a sequence for integration within the genome of a targeted cell. In some embodiments, the engineered nucleic acid is an mRNA. In some embodiments, therefore, strategic incorporation of the miRNA binding sequences in the mRNA sequence in the 5′ or 3′ UTR regions can negatively regulate the mRNA half-life, and therefore contribute to cell specific expression of the mRNA when the mRNA is administered in vivo.

In some embodiments the engineered nucleic acid sequence is optimized for expression in human. In some embodiments the engineered nucleic acid sequence is optimized for expression in a myeloid cell type in a human, wherein the desired target cell type in this case are the myeloid cells.

In some embodiments, the engineered nucleic acid is an engineered mRNA.

In some embodiments, the recombinant mRNA isolated mRNA comprises a sequence encoding a therapeutic protein or peptide.

In some embodiments, the recombinant mRNA is designed for expression in specific cell types, in preference to other cell types, e.g., in a cell population comprising heterogenous cell types ex vivo. In some embodiments, the recombinant mRNA is designed for expression in certain specific cell types in vitro or in vivo.

In some embodiments, cell specific expression may be achieved by incorporating one or more miRNA binding sites in 3′UTR of the recombinant mRNA for negative regulation of the mRNA in a cell that expresses the corresponding miRNA.

In some embodiments, a recombinant mRNA as is described in the above paragraphs is delivered to a cell ex vivo. In some embodiments, a recombinant mRNA as is described in the above paragraphs is delivered to a cell in vivo. The outer surface of a cell is directly contacted with the recombinant mRNA (e.g., an isolated recombinant mRNA or mRNA construct as described previously) for delivery of the mRNA to a cell ex vivo, whereupon the cell uptakes the recombinant mRNA, whereupon the mRNA is translated and the encoded protein or peptide is expressed inside the cell. The recombinant mRNA may be formulated in a lipid nanoparticle delivery vehicle (LNP). Such formulation may be used for in vivo delivery. In some embodiments, such formulation in lipid nanoparticles is designed for systemic delivery in vivo. For example, the recombinant mRNA is formulated for delivery to a cell inside a living organism via injection of a liquid formulation comprising the mRNA encapsulated in an LNP. In some embodiments, the miRNA targeting sites comprise 10-40 nucleotide residues that can form base pairing with the corresponding contiguous sequences in the miRNA strand. In some embodiments, the recombinant mRNA is designed to comprise 2, 3, 4, 5 copies of the miRNA target sites, i.e., miRNA binding sites. Hence, provided herein is a method and compositions for expressing an exogenous polypeptide from a recombinant polynucleic acid (e.g., an mRNA that is an isolated recombinant mRNA) in a specific cell, the method comprising contacting a heterogenous population of cells comprising the specific cell and at least another cell that is not the specific cell with a composition comprising the recombinant polynucleic acid comprising a sequence encoding the exogenous polypeptide, and one or more miRNA target sequences; wherein the exogenous polypeptide is expressed in the specific cell of the heterogenous population of cells, and wherein the exogenous polypeptide is not substantially expressed in the cell that is not the specific cell in the heterogenous population of cells. The miRNA target sites are designed such that the corresponding miRNAs are naturally expressed in a cell type that is not the specific cell, and where expression of the exogenous polypeptide encoded by the sequence within the mRNA is not desired; where the naturally occurring miRNA in the cell that is not the specific cell binds to the miRNA target site of the mRNA and degrades it. In some embodiments, the specific cell is a myeloid cell. In some embodiments, the cell that is not the specific cell type, where expression of the sequence encoding the exogenous polypeptide is nor required, is a non-myeloid cell, e.g., a neuron, an epithelial cell, a liver cell, a muscle cell, a lymphocyte etc. In some embodiments, a cell that is a non-myeloid cell is a cell of high abundance in the heterogenous cell population. In some embodiments, the non-myeloid cell can be a liver cell. In some embodiments, the non-myeloid cell is an epithelial cell.

In some embodiments, the mRNA comprises a sequence encoding an exogenous therapeutic polypeptide. In some embodiments, the therapeutic polypeptide is designed to be expressed preferentially in myeloid cells, a not in non-myeloid cells. For example, systemically administered nucleic acids such as a nucleic acid therapeutic is often up taken up by liver cells or epithelial cells. Where expression of the therapeutic is desired in myeloid cells and expression is desired to be avoided in epithelial cells for example, the mRNA is designed so as to comprise a miRNA target site for an miRNA that is readily present in an epithelial cell and not expressed in a myeloid cell.

For example, miRNA miR-10a is highly enriched in epithelial cells, in muscle cells, hepatic sinusoidal endothelial cells, and myeloid cells do not express miR-10a. For example, miR2-27b is highly enriched in liver cells (hepatocytes), and myeloid cells are relatively devoid of miR-27b. Likewise, miR-192 is enriched in hepatocytes, and its expression is nearly absent in myeloid cells. For example, miR-122 is highly expressed in hepatocytes with very little expression in myeloid cells. By “expression” of an miRNA it is understood that miRNAs are readily formed and are present in such cells. By “not expressed,” or “not readily expressed,” or “nearly absent” in case of an miRNA it may be understood that the miRNA is undetectable, or its effect on an mRNA having a target site for the miRNA is undetectable or both. In some embodiments, the expression of the exogenous polypeptide (or protein) encoded by the recombinant mRNA comprising the miRNA target sequence for an miRNA expressed in a cell other than the specific cell where expression is desired is reduced in the cell that is not the specific cell by at least 50%, at least 60%, at least 70%, at least 80%, at least 95% or more compared to that in the specific cell where expression of the same is desired.

In some embodiment, miRNA-122 (miR-122) binding sites are incorporated within the 3′ UTR of the mRNA for expression of a polypeptide encoded by a sequence in the mRNA specifically in a myeloid cell. A human miR-122 may comprise a sequence (NR_029667):

(SEQ ID NO: 93)
ccttagcagagctgtggagtgtgacaatgg tgtttgtgtctaaactatc
aaacgccattatcacactaaatagctactgctaggc.

In some embodiments, miRNA-192 (miR-192) binding sites are incorporated within the 3′ UTR of the mRNA for expression of a polypeptide encoded by a sequence in the mRNA specifically in a myeloid cell. A human miR-192 may comprise a sequence (NR_029578):

(SEQ ID NO: 1)
gccgagaccgagtgcacagggctctgacctatgaattgacagccagtgct
ctcgtctcccctctggctgccaattccataggtcacaggtatgttcgcct
caatgccagc

For example, a representative target sequence for the miRNA is: ggcugucaauucauaggucag (SEQ ID NO: 4), which may be included in the 3′ UTR of the recombinant mRNA construct. The sequence reverse complimentary to the target sequence is shown in SEQ ID NO: 1 by bold and underlining.

In some embodiments, miRNA-27b binding sites are incorporated within the 3′ UTR of the mRNA for expression of a polypeptide encoded by a sequence in the mRNA specifically in a myeloid cell. In some embodiments, a human miR27b comprises a sequence NR_029665:

(SEQ ID NO: 2)
acctctctaacaaggtgcagagcttagctgattggtgaacagtgattggt
ttccgctttgttcacagtggctaagttctgcacctgaagagaaggtg.

For example, a representative target sequence for the miRNA is guucaccaaucagcuaagcucu (SEQ ID NO: 6), which may be included in the 3′ UTR of the recombinant mRNA construct. The sequence reverse complimentary to the target sequence is shown in SEQ ID NO: 2 by bold and underlining.

In some embodiments, miR-10a binding sites are incorporated within the 3′ UTR of the mRNA for expression of a polypeptide encoded by a sequence in the mRNA specifically in a myeloid cell. In some embodiments, a human miR-10a comprises a sequence NR_029608:

(SEQ ID NO: 3)
gatctgtctgtcttctgtatataccctgtagatccgaatttgtgtaagga
attttgtggtcacaaattcgtatctaggggaatatgtagttgacataaac
actccgctct.

For example, a representative target sequence for the miRNA is cacaaauucggaucuacagggua (SEQ ID NO: 8), which may be included in the 3′ UTR of the recombinant mRNA construct. The sequence reverse complimentary to the target sequence is shown in SEQ ID NO: 3 by bold and underlining.

In one embodiment, incorporation of 1, 2, 3, 4 or more copies of miR-122 target sequences in an mRNA encoding a exogenous therapeutic polypeptide when contacted to a cell population comprising myeloid cells and non-myeloid cells, results in expression of the therapeutic polypeptide substantially in at least one myeloid cell, and does not express in an epithelial cell or a liver cell of the non-myeloid cells. In one embodiment, incorporation of 1, 2, 3, 4 or more copies of miR-122 target sequences in an mRNA encoding a exogenous therapeutic polypeptide in a formulation comprising a nanoparticle, e.g. a lipid nanoparticle when delivered via systemic route, results in expression of the therapeutic polypeptide substantially in at least one myeloid cell, and does not express in an epithelial cell or a liver cell.

In one embodiment, incorporation of 1, 2, 3, 4 or more copies of miR-192b target sequences in an mRNA encoding a exogenous therapeutic polypeptide when contacted to a cell population comprising myeloid cells and non-myeloid cells, results in expression of the therapeutic polypeptide substantially in at least one myeloid cell, and does not express in an epithelial cell or a liver cell of the non-myeloid cells. In one embodiment, incorporation of 1, 2, 3, 4 or more copies of miR-192b target sequences in an mRNA encoding a exogenous therapeutic polypeptide in a formulation comprising a nanoparticle, e.g. a lipid nanoparticle when delivered via systemic route, results in expression of the therapeutic polypeptide substantially in at least one myeloid cell, and does not express in an epithelial cell or a liver cell.

In one embodiment, incorporation of 1, 2, 3, 4 or more copies of miR-27b target sequences in an mRNA encoding a exogenous therapeutic polypeptide when contacted to a cell population comprising myeloid cells and non-myeloid cells, results in expression of the therapeutic polypeptide substantially in at least one myeloid cell, and does not express in an epithelial cell or a liver cell of the non-myeloid cells. In one embodiment, incorporation of 1, 2, 3, 4 or more copies of miR-27b target sequences in an mRNA encoding a exogenous therapeutic polypeptide in a formulation comprising a nanoparticle, e.g. a lipid nanoparticle when delivered via systemic route, results in expression of the therapeutic polypeptide substantially in at least one myeloid cell, and does not express in an epithelial cell or a liver cell.

In one embodiment, incorporation of 1, 2, 3, 4 or more copies of miR-Oa target sequences in an mRNA encoding a exogenous therapeutic polypeptide when contacted to a cell population comprising myeloid cells and non-myeloid cells, results in expression of the therapeutic polypeptide substantially in at least one myeloid cell, and does not express in an epithelial cell or a liver cell of the non-myeloid cells. In one embodiment, incorporation of 1, 2, 3, 4 or more copies of miR-10a target sequences in an mRNA encoding a exogenous therapeutic polypeptide in a formulation comprising a nanoparticle, e.g. a lipid nanoparticle when delivered via systemic route, results in expression of the therapeutic polypeptide substantially in at least one myeloid cell, and does not express in an epithelial cell or a liver cell.

In some embodiments, incorporation of an miRNA sequence as described above in the mRNA construct reduces expression of the polypeptide encoded by the miRNA in a non-myeloid cell by at least 50%, at least 60%, at least 70%, at least 80%, at least 90% or more. For example, the non-myeloid cell is a liver cell or an epithelial cell, and the miRNA target sequence on the mRNA binds to miR-192b. For example, the non-myeloid cell is a liver cell or an epithelial cell, and the miRNA target sequence on the mRNA binds to miR-122. For example, the non-myeloid cell is a liver cell or an epithelial cell, and the miRNA target sequence on the mRNA binds to miR-27b. For example, the non-myeloid cell is a liver cell or an epithelial cell, and the miRNA target sequence on the mRNA binds to miR-10a.

In some embodiments, an mRNA construct may be designed to comprise at least two miRNA target sequences described herein, wherein the miRNA target sequences are non-identical. In some embodiments, the at least two miRNA target sequences can hybridize with different regions of the same miRNA.

In some embodiments, the at least two miRNA target sequences can hybridize with at least two different miRNA sequences.

In one embodiment, an miRNA target sequence is a incorporated in the 3′UTR of an mRNA construct comprising a 3′UTR derived from a human alpha globin gene. In one embodiment, an miRNA target sequence is incorporated in the 3′ UTR of an mRNA construct comprising a 3′UTR derived from BGH.

Provided herein is a recombinant mRNA, e.g. an isolated recombinant mRNA comprising a sequence encoding a TROP2 binding CFP, and a 5′UTR and a 3′ UTR; wherein the 3′ UTR comprises a sequence that is at least 70%, at least 80% at least 90% identical to the 3′ UTR of a globin gene comprising at least one copy of any one of the miRNA target sequence of any one of SEQ ID Nos: 4-9 or miRNA target sequences highlighted in SEQ ID NO: 10. In some embodiments, the 5′ UTR is GGGAGACCCAAGCTGGCTAGCGTTTAAACTTAAGCTTGCCACC (SEQ ID NO: 94) (Underlined, Kozak sequence). In some embodiments, the 5′UTR comprises an ARCA cap. In some embodiments, the 3′UTR is from a BGH 3′ UTR, that has a sequence at least 70%, at least 80% at least 90% identical to the 3′ UTR

(SEQ ID NO: 73)
CTCGAGTCTAGAGGGCCCGTTTAAACCCGCTGATCAGCCTCGACTGTGCC
TTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGG
GCAGGACAGCAAGGGGGAGGATTGGGAAGACAATAGC

(Underlined, BGH poly A sequence). In some embodiments, a poly A tail is enzymatically added. In some embodiments, the 3′ UTR disclosed above further comprises 2 or more, 3 or more or 4 or more miRNA target sites selected from SEQ ID NOs 4-9 or miRNA target sequences highlighted in SEQ ID NO: 10 (none shown within the UTR sequence here). In some embodiments, the 3′ UTR disclosed above is further modified with addition of 2 or more, 3 or more or 4 or more miRNA target sites selected from SEQ ID NOs 4-9 or miRNA target sequences highlighted in SEQ ID NO: 10 (none shown within the UTR sequence here).

In some embodiments, the TROP2 binding CFP comprises a leader sequence MWLQSLLLLGTVACSIS (SEQ ID NO: 50); an extracellular scFv VH domain-linker-scFv-VL domain and a CD89 transmembrane domain. The TROP2 binding CFP comprises a sequence:

(SEQ ID NO: 95)
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSS

(scFv VH; CDR1, 2, and 3 sequentially underlined); linked to the VL domain via a linker having the sequence GGGGSGGGGSGGGGS (SEQ ID NO: 96), wherein the VL domain comprises a sequence

(SEQ ID NO: 97)
DIQLTQSPSSLSASVGDRVSITCKASQDVSIAVAWYQQKPGKAPKLLIY
SASYRYTGVPDRFSGSGSGTDFTLTISSLQPEDFAVYYCQQHYITPLTF
GAGTKVEIKR;

(CDR1, 2, and 3 sequentially underlined), which is associated with a sequence from CD89 comprising the TMD via a linker having the sequence, wherein the sequence from CD89 comprising the TMD has a sequence

(SEQ ID NO: 14)
DSIHQDYTTQNLIRMAVAGLVLVALLAILVENWHSHTALNKEASADVAEP
SWSQQMCQPGLTFARTPSVCK (TMD italicized).

Provided herein is a recombinant mRNA, e.g. an isolated recombinant mRNA comprising a sequence encoding a GPC3 binding CFP, and a 5′UTR and a 3′ UTR; wherein the 3′ UTR comprises a sequence that is at least 70%, at least 80% at least 90% identical to the 3′ UTR of a globin gene comprising at least one copy of any one of the miRNA target sequence of any one of SEQ ID NOs: 4-9 or miRNA target sequences highlighted in SEQ ID NO: 10.

In some embodiments, the 5′ UTR is AGGAGACCCAAGCTGGCTAGCGCCACC (SEQ ID NO: 98); (Underlined, Kozak sequence). In some embodiments, the 5′UTR comprises an Cap 1. In some embodiments, the 3′UTR is from a globin gene 3′ UTR, with multiple miR-27b targeting binding) sequences; wherein the 3′ UTR has a sequence

(SEQ ID NO: 99)
CTCGAGTAGGCTGGAGCCTCGGTGGCCATGCTTCTTGCCCCTTGGGCCTC
CCCCCAGCCCCTCCTCCCCTTCCTGCACCCGTACCCCCTTCACCAATCAG
CTAAGCTCTCGATTTCACCAATCAGCTAAGCTCTTCTAGATTCACCAATC
AGCTAAGCTCTTCACTTCACCAATCAGCTAAGCTCTGTGGTCTTTGAATA
AAGTCTGAGTGGGCGGCGAAGACAATAGC

(Underlined, miR-27b binding sequence). In some embodiments, a poly A tail is enzymatically added. In some embodiments, the UTR is further embedded with 2 or more, 3 or more or 4 or more additional miRNA target sites selected from SEQ ID Nos 4-9.

In some embodiments, the GPC3 binding CFP comprises a leader sequence MWLQSLLLLGTVACSIS (SEO ID NO: 50); an extracellular scFv VH domain-linker-scFv-VL domain and a CD89 transmembrane domain. The GPC3 binding CFP comprises a sequence:

(SEQ ID NO: 16)
QVQLVQSGAEVKKPGASVKVSCKASGYTFTDYEMHWVRQAPGQGLEWMG
ALDPKTGDTAYSQKFKGRVTLTADKSTSTAYMELSSLTSEDTAVYYCTR
FYSYTYWGQGTLVTVSS

(scFv VH, CDR1, 2, and 3 sequences sequentially underlined); linked to the VL domain by a linker having the sequence GGGGSGGGGSGGGGS (SEO ID NO: 96), wherein the VL domain comprises a sequence

(SEQ ID NO: 13)
DVVMTQSPLSLPVTPGEPASISCRSSQSLVHSNRNTYLHWYLQKPGQSPQ
LLIYKVSNRFSGVPDRFSGSGSGTDFTLKISRVEAEDVGVYYCSQNTHVP
PTFGQGTKLEIK;

(CDR1, 2, and 3 sequences sequentially underlined) which is associated with a sequence from CD89 comprising the TMD via a linker, wherein the sequence from CD89 comprising the TMD has a sequence

(SEQ ID NO: 14)
DSIHQDYTTQNLIRMAVAGLVLVALLAILVENWHSHTALNKEASADVAEP
SWSQQMCQPGLTFARTPSVCK (TMD italicized).

DNA, mRNA and Circular RNA: In some embodiments, naked DNA or messenger RNA (mRNA) may be used to introduce the nucleic acid inside the cell. In some embodiments, DNA or mRNA encoding the PFP is introduced into the phagocytic cell by lipid nanoparticle (LNP) encapsulation. mRNA is single stranded and may be codon optimized. In some embodiments the mRNA may comprise one or more modified or unnatural bases such as 5′-Methylcytosine, or Pseudouridine. mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. LNP encapsulated DNA or RNA can be used for transfecting myeloid cells, such as macrophages, or can be administered directly to a subject.

In some embodiments, circular RNA (circRNAs) encoding the PFP is used. In circular RNAs (circRNAs) the 3′ and 5′ ends are covalently linked, constitute a class of RNA. CircRNA may be delivered inside a cell or a subject using LNPs.

Delivery of Nucleic Acids into a Cell:

Nucleic acids encoding the CFP or PFP as described herein may be introduced to a cell, e.g. a myeloid cell, via different delivery approaches. A engineered nucleic acid as described herein may be introduced to a cell in vitro, ex vivo or in vivo. In some embodiments, a nucleic acid is introduced into a myeloid cell in the form of a plasmid or a vector. In some embodiments, the vector is a viral vector. In some embodiments, the vector is an expression vector, for example, a vector comprising one or more promoters, and other regulatory components, including enhancer binding sequence, initiation and terminal codons, a 5′ UTR, a 3′ UTR comprising a transcript stabilization element, optional conserved regulatory protein binding sequences and others. In some embodiments, the vector is a phage, a cosmid, or an artificial chromosome.

In some embodiments, a vector is introduced or incorporated in the cell by known methods of transfection, such as using lipofectamine, or calcium phosphate, or via physical means such as electroporation or nucleofection. In some embodiments the vector is introduced or incorporated in the cell by infection, a process commonly known as viral transduction.

In some embodiments, the vector for expression of the CFP is of a viral origin. In some embodiments, the engineered nucleic acid is encoded by a viral vector capable of replicating in non-dividing cells. In some embodiments, the nucleic acid encoding the engineered nucleic acid is encoded by a lentiviral vector, e.g. HIV and FIV-based vectors. In some embodiments the lentiviral vector is prepared in-house and manufactured in large scale for the purpose. In some embodiments, commercially available lentiviral vectors are utilized, as is known to one of skill in the art. In some embodiments, the engineered nucleic acid is encoded by a herpes simplex virus vector, a vaccinia virus vector, an adenovirus vector, or an adeno-associated virus (AAV) vector.

In some embodiments, a stable integration of transgenes into myeloid cells, such as macrophages, and other phagocytic cells may be accomplished via the use of a transposase and transposable elements, in particular, mRNA-encoded transposase. In one embodiment, Long Interspersed Element-1 (L1) RNAs may be contemplated for retrotransposition of the transgene and stable integration into myeloid cells, such as macrophages or phagocytic cells. Retrotransposon may be used for stable integration of a engineered nucleic acid encoding a phagocytic or tethering receptor (PR) fusion protein (PFP).

In some embodiments, the myeloid cell may be modified by expressing a transgene via incorporation of the transgene in a transient expression vector. In some embodiments expression of the transgene may be temporally regulated by a regulator from outside the cell. Examples include the Tet-on Tet-off system, where the expression of the transgene is regulated via presence or absence of tetracycline.

In some embodiments, the engineered nucleic acid described herein is a circular RNA (circRNA). A circular RNA comprises an RNA molecule where the 5′ end and the 3′ end of the RNA molecule are joined together. Without wishing to be bound by any theory, circRNAs have no free ends and may have longer half-life as compared to some other forms of RNAs or nucleic acid and may be resistant to digestion with RNase R exonuclease and turn over more slowly than its counterpart linear RNA in vivo. In some embodiments, the half-life of a circRNA is more than 20 hours. In some embodiments, the half-life of a circRNA is more than 30 hours. In some embodiments, the half-life of a circRNA is more than 40 hours. In some embodiments, the half-life of a circRNA is more than 48 hours. In certain embodiments, a circRNA comprises an internal ribosome entry site (IRES) element that engages a eukaryotic ribosome and an RNA sequence element encoding a polypeptide operatively linked to the IRES for insertion into cells in order to produce a polypeptide of interest.

CircRNAs can be prepared by methods known to those skilled in the art. For example, circRNAs may be chemically synthesized and/or enzymatically synthesized, for example by enzymatically synthesis of the RNA followed by chemical joining of the ends of the RNA to form the circRNA. In some embodiments, a linear primary construct or linear mRNA may be cyclized, or concatemerized to create a circRNA. The mechanism of cyclization or concatemerization may occur through methods such as, but not limited to, chemical, enzymatic, or ribozyme catalyzed methods. The newly formed 5′-/3′-linkage may be an intramolecular linkage or an intermolecular linkage. In some embodiments, a linear primary construct or linear mRNA may be cyclized, or concatemerized using the chemical method to form a circRNA. In the chemical method, the 5′-end and the 3′-end of the nucleic acid (e.g., linear primary construct or linear mRNA) contain chemically reactive groups that, when close together, form a new covalent linkage between the 5′-end and the 3′-end of the molecule. The 5′-end may contain a NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond. In some embodiments, a DNA or RNA ligase, e.g. a T4 ligase, may be used to enzymatically link a 5′-phosphorylated nucleic acid molecule (e.g., a linear primary construct or linear mRNA) to the 3′-hydroxyl group of a nucleic acid forming a new phosphorodiester linkage. In some embodiments, a linear primary construct or linear mRNA may be cyclized or concatemerized by using at least one non-nucleic acid moiety. For example, the at least one non-nucleic acid moiety may react with regions or features near the 5′ terminus and/or near the 3′ terminus of the linear primary construct or linear mRNA in order to cyclize or concatemerize the linear primary construct or linear mRNA. In some embodiments, a linear primary construct or linear mRNA may be cyclized or concatemerized due to a non-nucleic acid moiety that causes an attraction between atoms, molecules surfaces at, near or linked to the 5′ and 3′ ends of the linear primary construct or linear mRNA. For example, a linear primary construct or linear mRNA may be cyclized or concatemerized by intermolecular forces or intramolecular forces. Non-limiting examples of intermolecular forces. In some embodiments, a linear primary construct or linear mRNA may comprise a ribozyme RNA sequence near the 5′ terminus and near the 3′ terminus. In some embodiments, a circRNA may be synthesized by inserting DNA fragments into a plasmid containing sequences having the capability of spontaneous cleavage and self-circularization. In some embodiments, a circRNA is produced by making a DNA construct encoding an RNA cyclase ribozyme, expressing the DNA construct as an RNA, and then allowing the RNA to self-splice, which produces a circRNA free from intron in vitro. In some embodiments, a circRNA is produced by synthesizing a linear polynucleotide, combining the linear nucleotide with a complementary linking oligonucleotide under hybridization conditions, and ligating the linear polynucleotide.

The circRNA may be modified or unmodified. In some embodiments, the circRNA is chemically modified. For example, an A, G, U or C ribonucleotide of a circRNA may comprise chemical modifications. In some embodiments, any region of a circRNA, e.g. the coding region of the CFP or PFP, the flanking regions and/or the terminal regions may contain one, two, or more (optionally different) nucleoside or nucleotide modifications. In some embodiments, a modified circRNA introduced to a cell may exhibit reduced degradation in the cell, as compared to an unmodified circRNA. Modifications such as to the sugar, the nucleobase, or the internucleoside linkage (e.g. to a linking phosphate/to a phosphodiester linkage/to the phosphodiester backbone) are also encompassed. In some embodiments, one or more atoms of nucleobase, e.g. a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Additional modifications to circRNAs are described in US20170204422, the entire content of which is incorporated herein by reference.

In some embodiments, the circRNA is conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptide species, such as lipids, lectins, carbohydrates, vitamins, or cofactors.

In some embodiments, the circRNA is administered directly to tissues of a subject. Additional description of circRNAs in U.S. Pat. Nos. 5,766,903, 5,580,859, 5,773,244, 6,210,931, PCT publication No. WO1992001813, Hsu et al., Nature (1979) 280:339-340, Harland & Misher, Development (1988) 102:837-852, Memczak et al. Nature (2013) 495:333-338, Jeck et al., and RNA (2013) 19:141-157, each of which is incorporated herein by reference in its entirety.

In some embodiments, a nucleic acid is introduced into a myeloid cell with a nanoparticle (NP). A nanoparticle may be of various shapes or sizes and may harbor the nucleic acid encoding the CFP or PFP. In some embodiments, the NP is a lipid nanoparticle (LNP). In some embodiments, the NP comprises poly(amino acids), polysaccharides and poly(alpha-hydroxy acids), gold, silver, carbon, iron, silica, or any combination thereof. In some embodiments, the NP comprises a polylactide-co-glycolide (PGLA) particle. In some embodiments, the nucleic acid is encapsulated in the NP, for example, via water/oil emulsion or water-oil-water emulsion. In some embodiments, the nucleic acid is conjugated to the NP.

Nanoparticles (NPs) may be delivered to a cell in vitro, ex vivo or in vivo. In some embodiments, a NP is delivered to a phagocytic cell ex vivo. In some embodiments, a NP is delivered to a phagocytic cell in vivo. In some embodiments, the NP is less than 100 nm in diameter. In some embodiments, the NP is more than 100 nm in diameter. In some embodiments, the NP is a rod-shaped NP. In some embodiments, the NP is a spherical NP. In particular embodiments, the NP is a spherical NP for delivery to a phagocytic cell. In additional embodiments, the NP is at least 100 nm in diameter and does not trigger or triggers reduced toxicity when delivered to a cell.

In some embodiments, the NP is positively charged. In some embodiments, the NP is negatively charged. In some embodiments, the NP is neutral. In some embodiments, the NP is a cationic NP that is delivered and taken up by a myeloid cell ex vivo or in vivo.

Stiffness may affect the biological impact of NPs. NPs made of rigid materials may be associated with increased potential for embolism, while flexible polymer-based NPs that can more easily deform may gain better access to tissues during the complex vascular changes associated with inflammation. The fluidity of NPs, too, affects the ability of antigen-loaded NP to stimulate immune responses. Thus, intramuscular, solid-phase, antigen-containing liposome immunization may elicit a more robust Th1/Th17 response than similarly administered fluid-phase liposomes. Without wishing to be bound by any theory, solid-phase particles may result from the formation of an immobilized antigen particle depot and may result in a prolonged supply of antigen for APCs also associated with upregulation of positive costimulatory molecules such as CD80, which support efficient T cell priming.

In some embodiments, a protein corona may form around NPs. A protein corona may form in a two-step process. In the first step, high-affinity proteins rapidly bind to NPs to form a primary corona. In the second step, proteins of lower affinity bind either directly to the NP or to the proteins in the primary corona forming a secondary corona. Constituents of the protein corona may thus be impacted by the protein content of the serum and thus by the homeostatic or immune responses that regulate it. In some embodiments, proteins with high abundance, such as albumin, comprise a significant proportion of the primary corona. In some embodiments, NPs with different charges bind significant amounts of less-abundant proteins in particular environments, e.g. in plasma with certain antigen or antibody. In vivo formation of a protein corona may alter NP charge or mask functional groups important for NP targeting to certain receptors and/or enhance clearance of NPs by phagocytes. In some embodiments, NPs are engineered to reduce changes to NP charges or masking of functional groups, and/or increase the serum half-life of the NPs. In some embodiments, the NP comprises lipid anchored PEG moieties. In some embodiments, NP surface coatings are designed to modulate opsonization events. For example, the NP's surface may be coated with polymeric ethylene glycol (PEG) or its low molecular weight derivative polyethylene oxide (PEO). Without wishing to be bound by any theory, PEG increases surface hydrophilicity and can prevent NPs from merging with aqueous solutions. In some embodiments, the NP coated with PEG or PEO are engineered to result in reduced toxicity or increased biocompatibility of the NPs. Additional NP design and NP targeting for myeloid cells are described in Getts et al., Trends Immunol. 36(7): 419-427 (2015), the entirety of which is incorporated herein by reference. In some embodiments, the NP comprises an ionizable lipid, a sterol. In some embodiments, the NP comprises a PEG lipid, a phospholipid and cholesterol.

NPs described herein may be used to introduce the engineered nucleic acid into a cell in in vitro/ex vivo cell culture or administered in vivo. In some embodiments, the NP is modified for in vivo administration. For example, the NP may comprise surface modification or attachment of binding moieties to bind specific toxins, proteins, ligands, or any combination thereof, before being taken up by liver or spleen phagocytes. In recent rodent proof-of-concept studies, infused highly negatively charged ‘immune-modifying NPs’ (IMPs) can absorb certain blood proteins, including S100 family and heat shock proteins, before finally being removed and destroyed by cells of the mononuclear phagocyte system. Furthermore, this mechanism may also be used to capture and concentrate certain circulating proteins. IMPs have been shown to bind Annexin 1. The accumulation of Annexin 1 and its presentation to particular leukocyte subsets can have broad immune outcomes. For example, Annexin 1-loaded NPs may reduce neutrophils via induction of apoptosis and/or promote T cell activation. In some embodiments, the NP is designed to target a cell surface receptor, e.g. a scavenger receptor. In some embodiments, a NP is a particle with a negative surface charge.

In some embodiments, the NP encapsulates the nucleic acid wherein the nucleic acid is a naked DNA molecule. In some embodiments, the NP encapsulates the nucleic acid wherein the nucleic acid is an mRNA molecule. In some embodiments, the NP encapsulates the nucleic acid wherein the nucleic acid is a circular RNA (circRNA) molecule. In some embodiments, the NP encapsulates the nucleic acid wherein the nucleic acid is a vector, a plasmid, or a portion or fragment thereof.

In some embodiments, the NP is a Lipid nanoparticle (LNP). LNPs may comprise a polar and or a nonpolar lipid. In some embodiments cholesterol is present in the LNPs for efficient delivery. LNPs are 100-300 nm in diameter provide efficient means of mRNA delivery to various cell types, including myeloid cells, such as macrophages. In some embodiments, LNP may be used to introduce the nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiment described herein, lipid nanoparticles are formed associating or encapsulating the full length recombinant (engineered) mRNA. In some embodiments, the number of mRNA molecules per LNP is regulated for optimum delivery of the mRNA inside the cell. In some embodiments, the LNP is used to deliver mRNA systemically, that may be taken up by myeloid cells in vivo. In some embodiments, the LNP may comprise target moieties.

In some embodiments, the LNP does not comprise myeloid cell-targeting moieties on the surface, but the mRNA is designed for myeloid cell-specific expression. In some embodiments, the LNP encapsulates the nucleic acid wherein the nucleic acid is inserted in a vector, such as a plasmid vector. In some embodiments, the LNP encapsulates the nucleic acid wherein the nucleic acid is a circRNA molecule.

In some embodiments, mRNA can be encapsidated within mammalian retro-viral like PEG10 packages that deliver the mRNA inside a cell. Specific fusogens may be used for cell targeting with PEG10 delivery to organ, tissue or cells, e.g., myeloid cells. PEG10 is known to bind to its own mRNA and deliver it inside a cell. PEG10 UTR regions may be incorporated flanking the coding region of the mRNA, to facilitate PEG10 encapsidation. (Segel et al., Science (2021), 373: 6557, p 882-889).

In some embodiments, the LNP is used to deliver the nucleic acid into the subject. LNP can be used to deliver nucleic acid systemically in a subject. It can be delivered by injection. In some embodiments, the LNP comprising the nucleic acid is injected by intravenous route. In some embodiments the LNP is injected subcutaneously. In some embodiments the LNP is injected intramuscularly.

### Pharmaceutical Composition

Provided herein is a pharmaceutical composition, comprising engineered myeloid cells, such as macrophages, comprising an engineered nucleic acid encoding the CFP and a pharmaceutically acceptable excipient.

Also provided herein is a pharmaceutical composition, comprising a nucleic acid encoding the CFP and a pharmaceutically acceptable excipient. The pharmaceutical composition may comprise DNA, mRNA or circRNA or an LNP or a liposomal composition comprising any one of these.

Also provided herein is a pharmaceutical composition comprising a vector comprising the engineered nucleic acid encoding the CFP and a pharmaceutically acceptable excipient. The pharmaceutical composition may comprise DNA, mRNA or circRNA inserted in a plasmid vector or a viral vector.

In some embodiments the engineered myeloid cells, such as macrophages, are grown in cell culture sufficient for a therapeutic administration dose, and washed, and resuspended into a pharmaceutical composition.

In some embodiments the excipient comprises a sterile buffer, (e.g. HEPES or PBS) at neutral pH. In some embodiment, the pH of the pharmaceutical composition is at 7.5. In some embodiments, the pH may vary within an acceptable range. In some embodiments, the engineered cells may be comprised in sterile enriched cell suspension medium comprising complement deactivated or synthetic serum. In some embodiments the pharmaceutic composition further comprises cytokines, chemokines or growth factors for cell preservation and function.

In some embodiments, the pharmaceutical composition may comprise additional therapeutic agents, co-administered with the engineered cells.

### Treatment Methods

Provided herein are methods for treating cancer in a subject using a pharmaceutical composition comprising engineered myeloid cells, such as phagocytic cells (e.g., macrophages), expressing a engineered nucleic acid encoding a CFP, such as a phagocytic receptor (PR) fusion protein (PFP), to target, attack and kill cancer cells directly or indirectly. The engineered myeloid cells, such as phagocytic cells, are also designated as CAR-P cells in the descriptions herein.

Cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some aspects, any gene of interest can be expressed in a myeloid cell, such that the cell can be used to treat a disease that requires, for example an active phagocytic cell, such as an infection, where the myeloid cell may be specifically engineered to target, engulf and destroy the pathogen.

In general, cellular immunotherapy comprises providing the patient a medicament comprising live cells. In some aspects a patient or a subject having cancer, is treated with autologous cells, the method comprising, isolation of PBMC-derived myeloid cells, such as macrophages, modifying the cells ex vivo to generate phagocytic myeloid cells capable of tumor lysis by introducing into the cells a engineered nucleic acid encoding a CFP, and administering the modified myeloid cells into the subject.

In some aspects, a subject is administered one or more doses of a pharmaceutical composition comprising therapeutic myeloid cells, such as phagocytic cells, wherein the cells are allogeneic. An HLA may be matched for compatibility with the subject, and such that the cells do not lead to graft versus Host Disease, GVHD. A subject arriving at the clinic is HLA typed for determining the HLA antigens expressed by the subject.

HLA-typing is conventionally carried out by either serological methods using antibodies or by PCR-based methods such as Sequence Specific Oligonucleotide Probe Hybridization (SSOP), or Sequence Based Typing (SBT).

The sequence information may be identified by either sequencing methods or methods employing mass spectrometry, such as liquid chromatography-mass spectrometry (LC-MS or LC-MS/MS, or alternatively HPLC-MS or HPLC-MS/MS). These sequencing methods may be well-known to a skilled person and are reviewed in Medzihradszky K F and Chalkley R J. Mass Spectrom Rev. 2015 January-February; 34(1):43-63.

In some aspects, the phagocytic cell is derived from the subject, electroporated, transfected or transduced with the engineered nucleic acid in vitro, expanded in cell culture in vitro for achieving a number suitable for administration, and then administered to the subject. In some embodiments, the steps of electroporated, transfected or transduced with the engineered nucleic acid in vitro, expanded in cell culture in vitro for achieving a number suitable for administration takes 2 days, or 3 days, or 4 days or 5 days or 6 days or 7 days or 8 days or 9 days or 10 days.

In some embodiments, sufficient quantities of electroporated, transfected or transduced myeloid cells, such as macrophages, comprising the engineered nucleic acid are preserved aseptically, which are administered to the subject as “off the shelf” products after HLA typing and matching the product with the recipients HLA subtypes. In some embodiments, the engineered phagocytes are cryopreserved. In some embodiments, the engineered phagocytes are cryopreserved in suitable media to withstand thawing without considerable loss in cell viability.

In some embodiment, the subject is administered a pharmaceutical composition comprising the DNA, or the mRNA or the circRNA in a vector, or in a pharmaceutically acceptable excipient described above.

In some embodiments the administration of the off the shelf cellular products may be instantaneous, or may require 1 day, 2 days or 3 days or 4 days or 5 days or 6 days or 7 days or more prior to administration. The pharmaceutical composition comprising cell, or nucleic acid may be preserved over time from preparation until use in frozen condition. In some embodiments, the pharmaceutical composition may be thawed once. In some embodiments, the pharmaceutical composition may be thawed more than once. In some embodiments, the pharmaceutical composition is stabilized after a freeze-thaw cycle prior administering to the subject. In some embodiments the pharmaceutical composition is tested for final quality control after thawing prior administration.

In some embodiments, a composition comprising 10\hat{ }6 engineered cells are administered per administration dose. In some embodiments, a composition comprising 10\hat{ }7 engineered cells are administered per administration dose. In some embodiments, a composition comprising 5×10\hat{ }7 engineered cells are administered per administration dose. In some embodiments, a composition comprising 10\hat{ }8 engineered cells are administered per administration dose. In some embodiments, a composition comprising 2×10\hat{ }8 engineered cells are administered per administration dose. In some embodiments, a composition comprising 5×10\hat{ }8 engineered cells are administered per administration dose. In some embodiments, a composition comprising 10\hat{ }9 engineered cells are administered per administration dose. In some embodiments, a composition comprising 10\hat{ }10 engineered cells are administered per administration dose.

In some embodiments, the engineered myeloid cells, such as phagocytic cells, are administered once.

In some embodiments, the engineered myeloid cells, such as phagocytic cells, are administered more than once.

In some embodiments, the engineered myeloid cells, such as phagocytic cells, are twice, thrice, four times, five times, six times, seven times, eight times, nine times, or ten times or more to a subject over a span of time comprising a few months, a year or more.

In some embodiments, the engineered myeloid cells, such as phagocytic cells, are administered twice weekly.

In some embodiments, the engineered myeloid cells, such as phagocytic cells, are administered once weekly.

In some embodiments, the engineered myeloid cells, such as phagocytic cells, are administered once every two weeks.

In some embodiments, the engineered myeloid cells, such as phagocytic cells, are administered once every three weeks.

In some embodiments, the engineered myeloid cells, such as phagocytic cells, are administered once monthly.

In some embodiments, the engineered phagocytic cells are administered once in every 2 months, once in every 3 months, once in every 4 months, once in every 5 months or once in every 6 months.

In some embodiments, the engineered myeloid cells, such as phagocytic cells, are administered by injection. In some embodiments, the engineered myeloid cells, such as phagocytic cells, are administered by infusion. In some embodiments, the engineered myeloid cells, such as phagocytic cells, are administered by intravenous infusion. In some embodiments, the engineered myeloid cells, such as phagocytic cells, are administered by subcutaneous infusion.

The pharmaceutical composition comprising the engineered nucleic acid or the engineered cells may be administered by any route which results in a therapeutically effective outcome. These include, but are not limited to enteral (into the intestine), gastroenteral, epidural (into the dura mater), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavemosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

In some embodiments, the subject is administered a pharmaceutical composition comprising the nucleic acid encoding the CFP or PFP as described herein. In some embodiments, the subject is administered a pharmaceutical composition comprising DNA, mRNA, or circRNA. In some embodiments, the subject is administered a vector harboring the nucleic acid, e.g., DNA, mRNA, or circRNA. In some embodiments, the nucleic acid is administered or in a pharmaceutically acceptable excipient described above.

In some embodiments, the subject is administered a nanoparticle (NP) associated with the nucleic acid, e.g. a DNA, an mRNA, or a circRNA encoding the CFP or PFP as described herein. In some embodiments, the nucleic acid is encapsulated in the nanoparticle. In some embodiments, the nucleic acid is conjugated to the nanoparticle. In some embodiments, the NP is a polylactide-co-glycolide (PGLA) particle. In some embodiments, the NP is administered subcutaneously. In some embodiments, the NP is administered intravenously. In some embodiments, the NP is engineered in relation to the administration route. For example, the size, shape, or charges of the NP maybe engineered according to the administration route. In some embodiments, subcutaneously administered NPs are less than 200 nm in size. In some embodiments, subcutaneously administered NPs are more than 200 nm in size. In some embodiments, subcutaneously administered NPs are at least 30 nm in size. In some embodiments, the NPs are intravenously infused. In some embodiments, intravenously infused NPs are at least 5 nm in diameter. In some embodiments, intravenously infused NPs are at least 30 nm in diameter. In some embodiments, intravenously infused NPs are at least 100 nm in diameter. In certain embodiments, the administered NPs, e.g. intravenously administered NPs, are engulfed by circulating monocytes. Additional NP design and administration approaches are described in Getts et al., Trends Immunol. 36(7): 419-427 (2015), the entirety of which is incorporated herein by reference.

In some embodiments, the subject is administered a pharmaceutical composition comprising a circRNA encoding the CFP or PFP as described herein. The circRNA may be administered in any route as described herein. In some embodiments, the circRNA may be directly infused. In some embodiments, the circRNA may be in a formulation or solution comprising one or more of sodium chloride, calcium chloride, phosphate and/or EDTA. In some embodiment, the circRNA solution may include one or more of saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In some embodiments, the circRNA solution is lyophilized. The amount of each component may be varied to enable consistent, reproducible higher concentration saline or simple buffer formulations. The components may also be varied in order to increase the stability of circRNA in the buffer solution over a period and/or under a variety of conditions. In some embodiments, the circRNA is formulated in a lyophilized gel-phase liposomal composition. In some embodiments, the circRNA formulation comprises a bulking agent, e.g. sucrose, trehalose, mannitol, glycine, lactose and/or raffinose, to impart a desired consistency to the formulation and/or stabilization of formulation components. Additional formulation and administration approaches for circRNA as described in US Publications No. US2012060293, and US20170204422 are herein incorporated by reference in entirety.

In some embodiments, the subject is administered a pharmaceutical composition comprising a mRNA encoding the CFP or PFP as described herein. In some embodiments, the mRNA is co-formulated into nanoparticles (NPs), such as lipid nanoparticles (LNPs). For example, the LNP may comprise cationic lipids or ionizable lipids. In some embodiments, the mRNA is formulated into polymeric particles, for example, polyethyleneimine particles, poly(glycoamidoamine), ly(β-amino)esters (PBAEs), PEG particles, ceramide-PEGs, polyamindoamine particles, or polylactic-co-glycolic acid particles (PLGA). In some embodiments, the mRNA is administered by direct injection. In some embodiments, the mRNA is complexed with transfection agents, e.g. Lipofectamine 2000, jetPEI, RNAiMAX, or Invivofectamine.

In some embodiments, the mRNA may be a naked mRNA. In some embodiments, the mRNA mRNA may be modified. In some embodiments, the mRNA is unmodified. For example, the mRNA may be chemically modified. In some embodiments, nucleobases and/or sequences of the mRNA are modified to increase stability and half-life of the mRNA. In some embodiments, the mRNA is glycosylated. Additional mRNA modification and delivery approaches as described in Flynn et al., BioRxiv 787614 (2019) and Kowalski et al. Mol. Ther. 27(4): 710-728 (2019) are each incorporated herein by reference in its entirety.

In some embodiments the mRNA is modified, improved and optimized by AI assisted tools and algorithms. For example, LinerDesign may be used for improving an mRNA base sequence to optimize folding, expression and longevity of expression. In some embodiments the mRNA sequence further to optimization may be designed to comprise UTR sequences that contain 2, 3, 4 or more miRNA binding sequences that can control expression of the mRNA in certain cells thereby reducing off-target activity, for example, when administered as an mRNA therapeutic systemically. Some examples are provided for demonstration purposes.

### Kits

To give but a few examples, in some embodiments, inventive kits include, for example, biotin and/or streptavidin reagents suitable for labeling miRNAs. In some embodiments, such reagents achieve covalent attachment of the label (e.g., biotin or streptavidin) to an miRNA. In some embodiments, such reagents achieve non-covalent attachment of the label to an miRNA. For example, a labeling reagent may associate a label with an miRNA via hybridization. To give but one example, in some embodiments, inventive kits comprise a means for attaching a standard sequence element to an miRNA (e.g., via expression of the miRNA in a vector containing the sequence element, direct linkage of a nucleic acid fragment containing the sequence element, etc.), and further comprise a label attached to the complement of the sequence element.

In some embodiments, inventive kits comprise one or more of a reverse transcriptase enzyme, deoxyribonucleotides, DNA polymerase (e.g., thermostable DNA polymerase), chain-terminating nucleotides, detectable (e.g., fluorescent, radioactive) nucleotides, one or more buffers, distilled water, etc.

In some embodiments, the present invention provides kits or compositions containing one or more agents that regulates mRNA levels; in some such embodiments, the present invention provides kits or compositions containing one or more agents that regulate levels of one or more miRNA targets. In some embodiments, such a kit or compositions may include one or more siRNA, for example targeting a specific miRNA target.

In certain embodiments, inventive such kits contain all of the components necessary to perform a relevant assay (e.g., detection assay, regulation assay), including all controls, directions for performing assays, and any necessary software for analysis and presentation of results. In some embodiments, components of inventive kits are provided in individual containers and multiple such containers are provided together in a common housing.

### EXAMPLES

### Example 1. Exemplary Sequences for Designing an Anti-CD5-CAR Encoding mRNA Construct with Selective Cell-Specific Expression

In this section, exemplary designs are contemplated for selective expression of a construct in a myeloid cell, when the construct is delivered to a cell population comprising myeloid cells and non-myeloid cells. Expression of anti-CD5 CAR is described as an exemplary CAR with the UTR modifications as described below. However, the invention can be carried on by replacing anti-CD5 CAR with any other known coding sequences, e.g., an anti-HER2 CAR sequence, which can be performed without additional invention. The prospective exemplary construct is an mRNA construct, Construct #1, in which miRNA 192 target sequence (miR-192-5p) sequence (SEQ ID NO: 4), is incorporated in the 5′-UTR within the mRNA. SEQ. ID NO: 4 is reverse complement of nucleic acid residues 24-44 of SEQ ID NO: 1. Exemplary construct has a 5′UTR, a sequence encoding an anti-CD5-CAR and a 3′UTR. The 5′-UTR comprises a 5′ methyl guanylate cap. The 5′-UTR is at least 45 nucleotides in length. The 3′-UTR is terminated by a sequence of 200 adenylate residues (poly A). The exemplary (hypothetical) construct #1 thus has the following structural elements from 5′-3′ orientation: 5′-methyl guanylate cap; miR-192p-target sequence SEQ ID NO: 4; anti-CD5-CAR coding sequence; 3′UTR; poly A sequence-3′. The mRNA construct may contain linker sequences in between any two adjacent structural elements (e.g., 5′-methyl guanylate cap and miR-192p-target sequence SEQ ID NO: 4).

In another exemplary design, Construct #2, an miRNA192b target sequence (miR-192-3p) sequence (SEQ ID NO: 5), is incorporated in the 3′-UTR within the mRNA. The exemplary Construct #2 has the following structural elements from 5′-3′ orientation: 5′-methyl guanylate cap; anti-CD5-CAR coding sequence; 3′UTR comprising miR-192-3p-target sequence (SEQ ID NO: 5); poly A sequence-3′. As described previously, and can be applied to other examples following, unless otherwise mentioned, each of the mRNA constructs described herein may contain linker sequences in between any two adjacent structural elements. A linker may be 3-100 nucleotides long.

An exemplary Construct #3 has the following structural elements from 5′-3′ orientation: 5′-methyl guanylate cap; miR-27-5p-target sequence SEQ ID NO: 6 in 5′UTR; anti-CD5-CAR coding sequence; 3′UTR; poly A sequence-3′. The mRNA construct may contain linker sequences in between any two adjacent structural elements.

In another exemplary design, Construct #4, an miRNA27b target sequence (miR-27-3p) sequence (SEQ ID NO: 7), is incorporated in the 3′-UTR within the mRNA. The exemplary Construct #4 has the following structural elements from 5′-3′ orientation: 5′-methyl guanylate cap; anti-CD5-CAR coding sequence; 3′UTR comprising miR-27-3p-target sequence SEQ ID NO: 7; poly A sequence-3′.

In another exemplary design, Construct #5, an miRNA10a target sequence (miR-10a-5p) sequence (SEQ ID NO: 8), is incorporated in the 5′-UTR within the mRNA. The exemplary Construct #5 has the following structural elements from 5′-3′ orientation: 5′-methyl guanylate cap; miR-10-5p-target sequence SEQ ID NO: 8 in 5′UTR; anti-CD5-CAR coding sequence; 3′UTR; poly A sequence-3′.

In another exemplary design, Construct #6, an miRNA10a target sequence (miR-10a-3p) sequence (SEQ ID NO: 9), is incorporated in the 3′-UTR within the mRNA. The exemplary Construct #6 has the following structural elements from 5′-3′ orientation: 5′-methyl guanylate cap; anti-CD5-CAR coding sequence; 3′UTR comprising miR-27-3p-target sequence SEQ ID NO: 9; poly A sequence-3′.

Another exemplary design, an mRNA construct, Construct #7 is disclosed herein, in which an miRNA 192 target sequence (miR-192-5p) sequence (SEQ ID NO: 4), is incorporated in the 5′-UTR within the mRNA and the miRNA27b target sequence (miR-27-3p) sequence (SEQ ID NO: 7), is incorporated in the 3′-UTR within the mRNA. The exemplary construct #7 has the following structural elements from 5′-3′: 5′-methyl guanylate cap; miR-192p-target sequence SEQ ID NO: 4; anti-CD5-CAR coding sequence; 3′UTR comprising miR-27-3p-target sequence SEQ ID NO: 7; poly A sequence-3′.

Yet another exemplary design, an mRNA construct, Construct #8 is disclosed herein, in which an miRNA 192 target sequence (miR-192-5p) sequence (SEQ ID NO: 4), is incorporated in the 5′-UTR within the mRNA and the miRNA27b target sequence (miR-27-3p) sequence (SEQ ID NO: 7) and the miRNA10a target sequence (miR-10a-3p) sequence (SEQ ID NO: 9), are incorporated in the 3′-UTR within the mRNA. The exemplary construct #8 has the following structural elements from 5′-3′: 5′-methyl guanylate cap; miR-192p-target sequence SEQ ID NO: 4; anti-CD5-CAR coding sequence; 3′UTR comprising miR-27-3p-target sequence SEQ ID NO: 7 and the miRNA10a target sequence (miR-10a-3p) sequence (SEQ ID NO: 9); and the poly A sequence-3′.

Yet another exemplary mRNA construct design is shown: comprising target sequences of two miR-sequences in the 5′UTR. Exemplary construct #9 has the following structural elements from 5′-3′: 5′-methyl guanylate cap; miR-192p-target sequence SEQ ID NO: 4; miR-27-5p-target sequence SEQ ID NO: 6 in 5′UTR; anti-CD5-CAR coding sequence; 3′UTR comprising the miRNA10a target sequence (miR-10a-3p) sequence (SEQ ID NO: 9); and the poly A sequence-3′.

Any combinations of the above, including further additions and replacements of elements described herein as may be easily conceived by a skilled artisan is contemplated within the disclosure. The miRNA target sequences discussed are provided in the table below.

TABLE 2
miRNA Target Sequences and Respective miRNA Binding Sites

| Target | | |
|---|---|---|
| Sequence | | Binds to human miRNA (sequence ID |
| Name | miRNA target sequence | provided) at nucleic acid residues: |
| miR-192- | ggcugucaauucauaggucag (SEQ ID | Nucleic acids 24-44 of SEQ ID NO: 1 |
| 5p | NO: 4) | |
| miR-192- | cugugaccuauggaauuggcag (SEQ ID | Nucleic acids 67-88 of SEQ ID NO: 1 |
| 3p | NO: 5) | |
| miR-27b- | guucaccaaucagcuaagcucu (SEQ ID | Nucleic acids 19-40 of SEQ ID NO: 2 |
| 5p | NO: 6) | |
| miR-27b- | gcagaacuuagccacugugaa (SEQ ID | Nucleic acids 61-81 of SEQ ID NO: 2 |
| 3p | NO: 7) | |
| miR-10a-5p | cacaaauucggaucuacagggua (SEQ ID | Nucleic acids 22-44 of SEQ ID NO: 3 |
| | NO: 8) | |
| miR-10a-3p | uauuccccuagauacgaauuug (SEQ ID | Nucleic acids 63-84 of SEQ ID NO: 3 |
| | NO: 9) | |

### Example 2. Formulation and Delivery of mRNA Constructs

mRNA construct designs described above are prepared and formulated for in vivo delivery. An exemplary construct for testing cell specific expression in several tissues of the experimental mouse model may be generated by tagging the anti-CD5-CAR coding region with a GFP encoding sequence without a stop codon in between anti-CD5-CAR sequence and the GFP coding sequence. In vitro transcribed mRNA is purified by HPLC and encapsulated into a lipid nanoparticle. Methods and compositions for generating lipid nanoparticle encapsulated (LNP) mRNA are known and described previously. Briefly, the LNP composition comprises a cationic lipid, and at least one neutral lipid. LNPs have a diameter of less than 200 nm. Nanoparticles are delivered via intravenous injection.

Upon delivery to experimental mouse model, mRNA expression will be tested in cells of various tissues and organs, hepatocytes at different time periods. Mice cohorts will be tested upon euthanasia at 72 hours after delivery and between 4-10 days. For example, a mouse model can be established for testing the expression of the anti-CD5 CARs in hepatocytes at 48, 72, 96 hours post injection. Hepatocytes uptake most of foreign nucleic acids delivered in an organisms. Expression of GFP can be used as surrogate for the expression of the anti-CD5-CAR. If miR-192b—which is copiously expressed in hepatocytes can bind to the miR-192-target sequences incorporated in the UTR regions, then the mRNA construct will not be translated, and/or the mRNA construct will be degraded by the action of the miR-192b. As per the rationale, expression of the anti-CD5-CAR will not be detectable in hepatocytes, or will be detected at a significantly lower amount than counterpart control mice, which are injected with a construct that lacks the miRNA target sequences but is otherwise identical to the test construct.

miR-192b is also expressed in high levels in epithelial cells. Expression of the anti-CD5-CAR will not be detectable in epithelial cells.

Constructs 1, 2, 7, 8 or 9, having miR-192b target sequences incorporated in the 5′- or 3′UTR—will be acted upon by miR-192b expressed in hepatocytes and epithelial cells will not express ant-CD5 CAR of the constructs.

### Example 3. Exploiting miRNA 122-5p Binding Sites in Globin mRNA UTRs for In Vivo Expression Constructs

In this example, alpha-Globin 3′ UTR was used to prepare exemplary mRNA constructs comprising a sequence encoding GFP to test GFP protein expression in myeloid cells—wherein the a-globin (hereafter globin) UTR is further engineered to comprise multiple miR122-5p binding sequences. miRNA 122 is abundant in hepatocytes. To test whether miR122-5p binding sites within the 3′UTR of the constructs can suppress expression of the GFP-encoding mRNA in hepatocytes, an exemplary globin 3′UTR tested has the sequence: CTCGAGTGATAATAGGCTGGAGCCTCGGTGGCCATGCTTCTTGCCCCTTGGGCCTCCCC CCAGCCCCTCCTCCCCTTCCTGCACCCGTACCCCCcaaacaccattgtcacactccaCGATcaaacaccat tgtcacactccaTCTAGAcaaacaccattgtcacactccaTCACcaaacaccattgtcacactccaGTGGTCTTTGAAT AAAGTCTGAGTGGGCGGCGAAGACAATAGC (SEQ ID NO: 10). In this sequence, 4 (four) miR122-5p binding sites are inserted within the globin 3′UTR, and the miR122-5p binding sites are marked in bold and lower case alphabets.

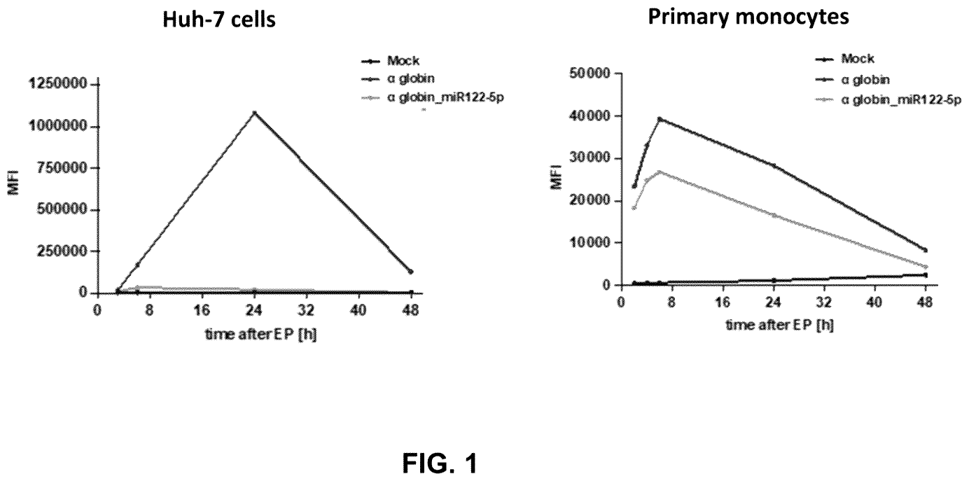

When Huh-7 cells, a human male hepatoma cell line was electroporated with an mRNA construct encoding GFP and comprising the 3′UTR (SEQ ID NO: 10), expression of the GFP was completely suppressed compared to transfection with a construct comprising a construct with globin 3′UTR that was not engineered to incorporate the miR122-5p binding sites (FIG. 1, left graph). In contrast, primary monocytes expressing the same construct showed significant expression of the encoded protein despite the presence of the miRNA binding sequence (FIG. 1, right graph). These results were highly encouraging and offer great mechanism for controlling expression of a construct specifically in one cell by design, in such circumstances where the construct is added to a mixed population of cells containing a variety of cell types, such as in an in vivo condition.

### Example 4. Construction of mRNA Encoding a CFP that Binds to GPC3 Antigen, with an Engineered UTR Having miRNA Binding Sequences

Based on the encouraging data above, an anti-GPC3 binding CFP construct was generated, with different miRNA binding sites for suppression of mRNA expression in hepatocytes, as listed as follows in Table 3, and tested.

TABLE 3
List of novel miRNA binding sites tested for suppression
of mRNA expression in hepatocytes.

| | # of binding sites | Coding sequence |
|---|---|---|
| miRNA | inserted into 3′UTR | tested |
| miR192-5p | 4 | GC33-CD89 |
| miR192-3p | 4 | GC33-CD89 |
| miR27b-5p | 4 | GC33-CD89 |
| miR27b-3p | 4 | GC33-CD89 |
| miR10a-5p | 4 | GC33-CD89 |
| miR10a-3p | 4 | GC33-CD89 |

GC33-CD89 stands for an anti-GPC3 binding CFP that comprises a variable heavy chain and a variable light chain domains connected by a short linker sequence, e.g. GGGGSGGGGSGGGGS (SEQ ID NO: 96); and is fused with a CD89 hinge, CD89 transmembrane and a short CD89 intracellular domain. In some of these experiments, an anti-GPC3 binding CFP comprises an amino acid sequence:

(SEQ ID NO: 11)
MWLQSLLLLGTVACSISQVQLVQSGAEVKKPGASVKVSCKASGYTFTDYE
MHWVRQAPGQGLEWMGALDPKTGDTAYSQKFKGKATLTADKSTSTAYMEL
SSLTSEDTAVYYCTRFYSYTYWGQGTLVTVSSGGGGSGGGGSGGGGSDVV
MTQSPLSLPVTPGEPASISCRSSQSLVHSNRNTYLHWYLQKPGQSPQLLI
YKVSNRFSGVPDRFSGSGSGTDFTLKISRVEAEDVGVYYCSQNTHVPPTF
GQGTKLEIKGSGGSDSIHQDYTTQNLIRMAVAGLVLVALLAILVENWHSH
TALNKEASADVAEPSWSQQMCQPGLTFARTPSVCK,

which contains the following domains, and VH-CDR1, CDR2 and CDR3 are the VL CDR1, 2 and 3 are marked by underlining respectively.

TABLE 4
Sequences of respective domains of the anti-GPC3 construct of SEQ ID NO: 11

| Segment/domain | Sequence |
|---|---|
| Signal peptide sequence | MWLQSLLLLGTVACSIS (SEQ ID NO: 50) |
| Variable Cheavy chain | QVQLVQSGAEVKKPGASVKVSCKASGYTFTDYEMHWVRQAP |
| domain (VH) | GQGLEWMGALDPKTGDTAYSQKFKGKATLTADKSTSTAYMEL |
| | SSLTSEDTAVYYCTRFYSYTYWGQGTLVTVSS (SEQ ID NO: 12) |
| Variable light chain | DVVMTQSPLSLPVTPGEPASISCRSSQSLVHSNRNTYLHWYLQK |
| domain (VL) | PGQSPQLLIYKVSNRFSGVPDRFSGSGSGTDFTLKISRVEAEDVG |
| | VYYCSQNTHVPPTFGQGTKLEIK (SEQ ID NO: 13) |
| CD89 extracellular hinge, | DSIHQDYTTQNLIRMAVAGLVLVALLAILVENWHSHTALNKEASA |
| Transmembrane (italicized) | DVAEPSWSQQMCQPGLTFARTPSVCK (SEQ ID NO: 14) |
| and intracellular domains | |

In some of these experiments, an anti-GPC3 binding CFP comprises an amino acid sequence:

(SEQ ID NO: 15)
MWLQSLLLLGTVACSISQVQLVQSGAEVKKPGASVKVSCKASGYTFTDYE
MHWVRQAPGQGLEWMGALDPKTGDTAYSQKFKGRVTLTADKSTSTAYMEL
SSLTSEDTAVYYCTRFYSYTYWGQGTLVTVSSGGGGSGGGGSGGGGSDVV
MTQSPLSLPVTPGEPASISCRSSQSLVHSNRNTYLHWYLQKPGQSPQLLI
YKVSNRFSGVPDRFSGSGSGTDFTLKISRVEAEDVGVYYCSQNTHVPPTF
GQGTKLEIKGSGGSDSIHQDYTTQNLIRMAVAGLVLVALLAILVENWHSH
TALNKEASADVAEPSWSQQMCQPGLTFARTPSVCK,

which contains the following domains, and VH-CDR1, CDR2 and CDR3 are the VL CDR1, 2 and 3 are marked by underlining respectively.

TABLE 5
Sequences of respective domains of the anti-GPC3 construct of SEQ ID NO: 15

| Segment/domain | Sequence |
|---|---|
| Signal peptide sequence | MWLQSLLLLGTVACSIS (SEQ ID NO: 50) |
| Variable heavy chain | QVQLVQSGAEVKKPGASVKVSCKASGYTFTDYEMHWVRQAPG |
| domain (VH) | QGLEWMGALDPKTGDTAYSQKFKGRVTLTADKSTSTAYMELSS |
| | LTSEDTAVYYCTRFYSYTYWGQGTLVTVSS (SEQ ID NO: 16) |
| Variable light chain | DVVMTQSPLSLPVTPGEPASISCRSSQSLVHSNRNTYLHWYLQKP |
| domain (VL) | GQSPQLLIYKVSNRFSGVPDRFSGSGSGTDFTLKISRVEAEDVGV |
| | YYCSQNTHVPPTFGQGTKLEIK (SEQ ID NO: 17) |
| CD89 extracellular hinge, | DSIHQDYTTQNLIRMAVAGLVLVALLAILVENWHSHTALNKEASA |
| Transmembrane (italicized) | DVAEPSWSQQMCQPGLTFARTPSVCK (SEQ ID NO: 18) |
| and intracellular domains | |

In some of these experiments, an anti-GPC3 binding CFP comprises 3′UTR having an alpha-globin 3′UTR sequence with four miR192-5p binding sites that suppress mRNA expression in hepatocytes but not in primary monocytes. For instance, the 3′UTR has a sequence CTCGAGTGATAATAGGCTGGAGCCTCGGTGGCCATGCTTCTTGCCCCTTGGGCCTCC CCCCAGCCCCTCCTCCCCTTCCTGCACCCGTACCCCCggctgtcaattcataggtcagCGATggctgt caattcataggtcagTCTAGAggctgtcaattcataggtcagTCACggctgtcaattcataggtcagGTGGTCTTTGAA TAAAGTCTGAGTGGGCGGCGAAGACAATAGC (SEQ ID NO: 19). The miR192-5p binding sequences are marked as lower case alphabets.

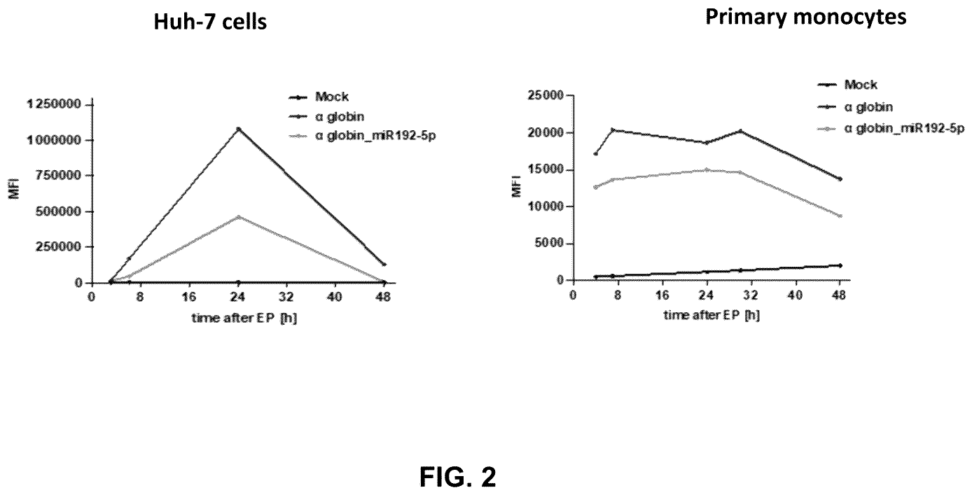

FIG. 2 demonstrates the effects of the miRNA binding site on its expression in hepatocytes (left graph) as well as primary monocytes (right graph). In these cases the mRNA was constructed with the engineered globin 3′UTR with miR192-5p binding sites. The data indicate a similar trend as with data in case of constructs with miRNA binding site for miR122-5p in FIG. 1, there was a higher suppression of expression of the mRNA encoded polypeptide in hepatocyte (left graph) compared to in primary monocytes.

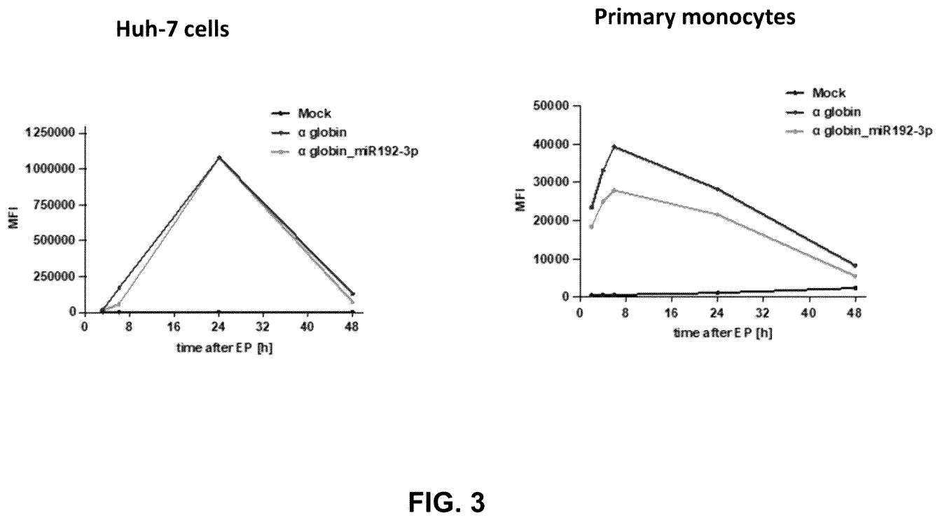

However, no effect was evident on polypeptide expression in case of the 3′UTR constructs having globin 3′UTR with four miR192-3p binding sites (FIG. 3) (sequence provided below).

CTCGAGTGATAATAGGCTGGAGCCTCGGTGGCCATGCTTCTTGCCCCTTGGGCCT CCCCCCAGCCCCTCCTCCCCTTCCTGCACCCGTACCCCCctgtgacctatggaattggcaggCGATctgtg acctatggaattggcaggTCTAGActgtgacctatggaattggcaggTCACctgtgacctatggaattggcaggGTGGTCTTTG AATAAAGTCTGAGTGGGCGGCGAAGACAATAGC (SEQ ID NO: 20). miR192-3p binding sites represented as lower case letter.

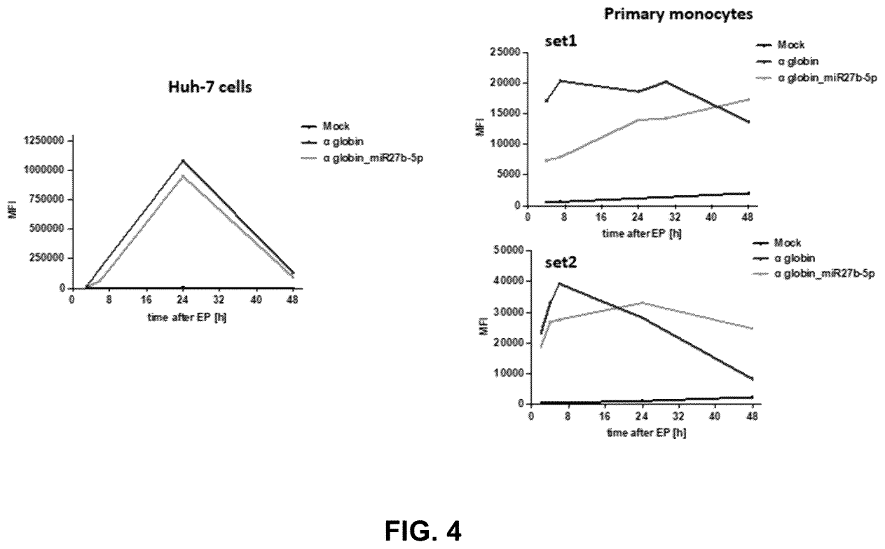

On the other hand, globin 3′UTR with miR27b-5p binding sites showed a surprising effect of prolonging mRNA expression of the mRNA encoded polypeptide, where the mRNA was constructed with the engineered globin 3′UTR with miR27b-5p binding sites. (FIG. 4, UTR sequence of SEQ ID NO: 21). The construct contained a globin 3′UTR with miR27b-5p binding sites having the sequence:

CTCGAGTGATAATAGGCTGGAGCCTCGGTGGCCATGCTTCTTGCCCCTTGGGCCT CCCCCCAGCCCCTCCTCCCCTTCCTGCACCCGTACCCCCttcaccaatcagctaagctctCGATttcaccaa tcagctaagctctTCTAGAttcaccaatcagctaagctctTCACttcaccaatcagctaagctctGTGGTCTTTGAATAAAG TCTGAGTGGGCGGCGAAGACAATAGC (SEQ ID NO: 21). miR27b-5p binding sites represented as lower case letter.

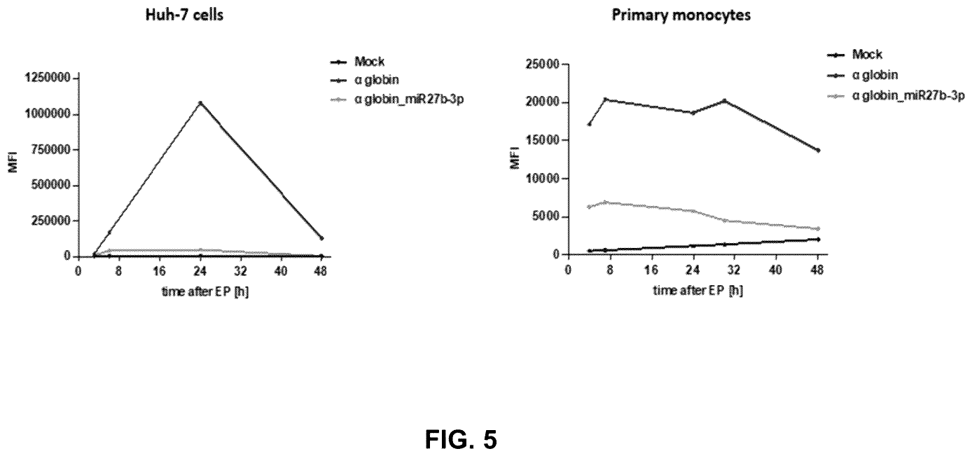

mRNA having engineered globin 3′UTR with four miR27b-3p binding sites did not exhibit any cell-specific advantage for monocyte cells. (FIG. 5). The respective engineered globin 3′UTR has the sequence: CTCGAGTGATAATAGGCTGGAGCCTCGGTGGCCATGCTTCTTGCCCCTTGGGCCTCCCC CCAGCCCCTCCTCCCCTTCCTGCACCCGTACCCCCgcagaacttagccactgtgaaCGATgcagaacttagc cactgtgaaTCTAGAgcagaacttagccactgtgaaTCACgcagaacttagccactgtgaaGTGGTCTTTGAATAAAG TCTGAGTGGGCGGCGAAGACAATAGC (SEQ ID NO: 22), where miR27b-3p miRNA binding sites are marked with lower case letters.

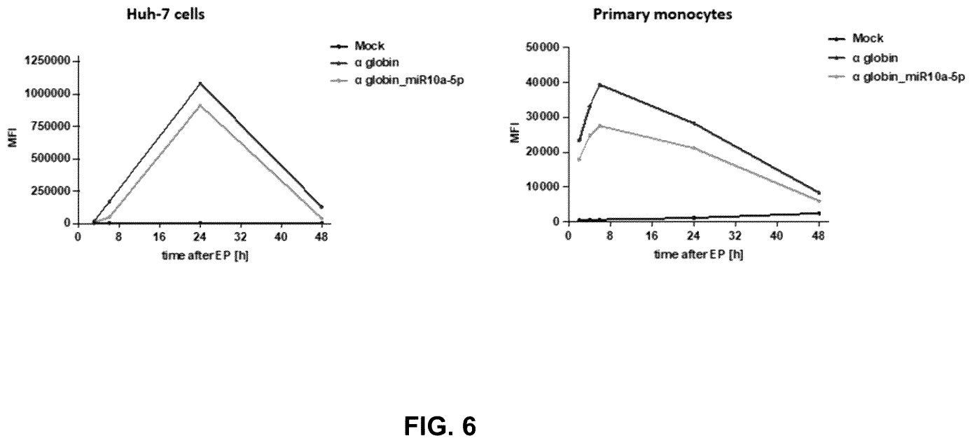

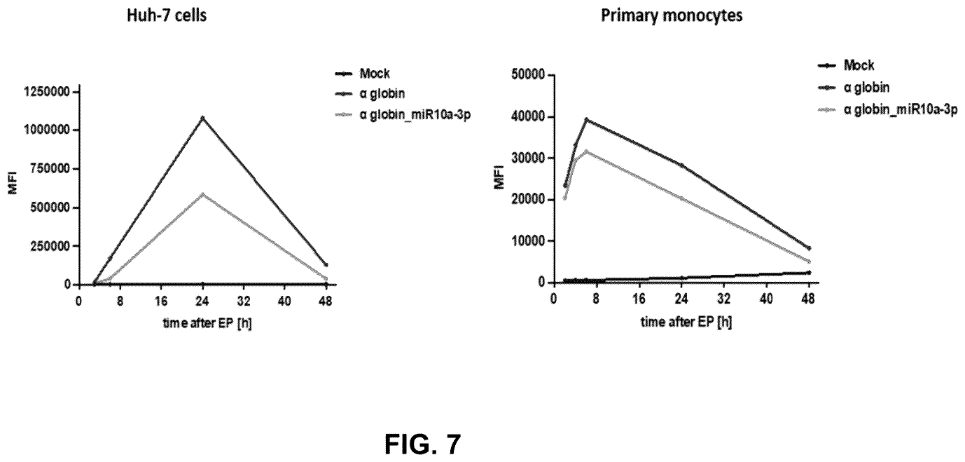

mRNA having engineered globin 3′UTR with four miR10a-5p binding sites and mRNA having engineered globin 3′UTR with four miR10a-3p binding sites also did not exhibit any cell-specific advantage for monocyte cells. (FIG. 6 and FIG. 7 respectively). The respective engineered globin 3′UTR with four miR10a-5p binding sites has the sequence: CTCGAGTGATAATAGGCTGGAGCCTCGGTGGCCATGCTTCTTGCCCCTTGGGCCTCCCC CCAGCCCCTCCTCCCCTTCCTGCACCCGTACCCCCcacaaattcggatctacagggtaCGATcacaaattcgg atctacagggtaTCTAGAcacaaattcggatctacagggtaTCACcacaaattcggatctacagggtaGTGGTCTTTGAATA AAGTCTGAGTGGGCGGCGAAGACAATAGC (SEQ ID NO: 23); and the engineered globin 3′UTR with four miR10a-3p binding sites has the sequence: CTCGAGTGATAATAGGCTGGAGCCTCGGTGGCCATGCTTCTTGCCCCTTGGGCCTCCCC CCAGCCCCTCCTCCCCTTCCTGCACCCGTACCCCCtattcccctagatacgaatttgCGATtattcccctagata cgaatttgTCTAGAtattcccctagatacgaatttgTCACtattcccctagatacgaatttgGTGGTCTTTGAATAAAGTC TGAGTGGGCGGCGAAGACAATAGC (SEQ ID NO: 24), miR10a-5p and miR10a-3p binding sites in each case are represented by lower case letters.

Taken together, there is a strong possibility of further optimizing mRNA sequences including the coding sequences and the UTRs, for providing improved therapeutic mRNA molecules encoding a polypeptides of interest and selectively express in myeloid cells and at the same time suppress expression of the encoded polypeptides in other cells, principally in liver cells when the mRNA molecule is administered systemically in vivo. Most nucleic acid materials delivered systemically accumulate and express in the liver cells and are not transported to other tissues and organs, and the above strategies may provide remedy for that.

### Example 5. AI Based mRNA Sequence Optimization for Stability, Prolonged Expression

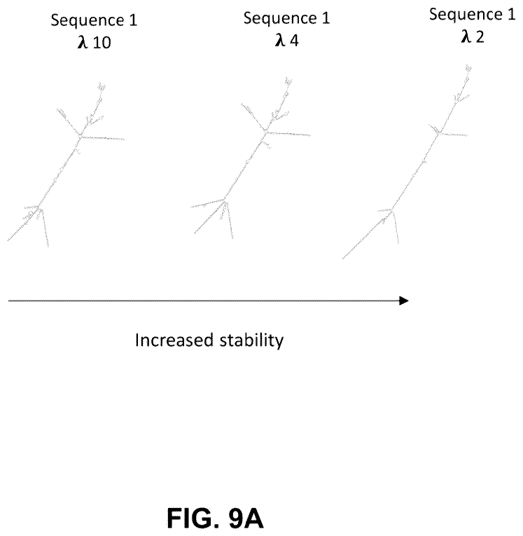

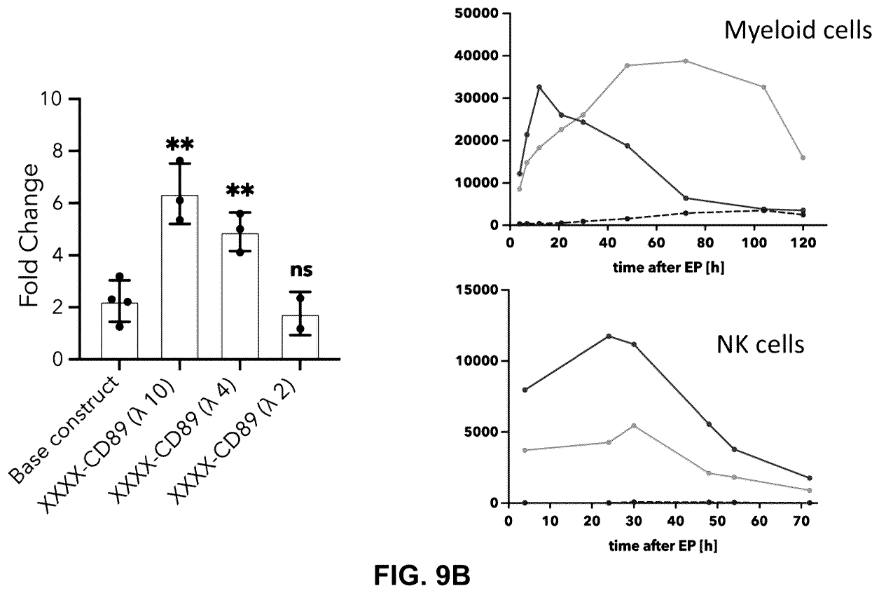

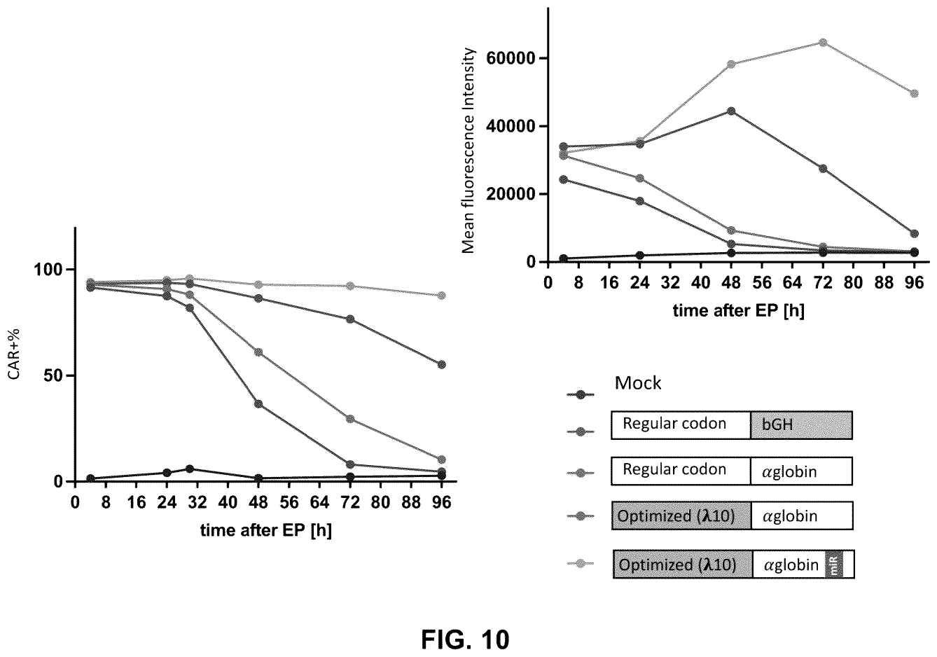

Using Artificial Intelligence based tools for codon optimization parameters, mRNA coding sequences were further optimized for more robust and prolonged expression of mRNA encoded polypeptides of interest in myeloid cells. LinearDesign algorithms are experimented over several to evaluate improved expression of the encoded polypeptides in myeloid cells. Optimization was carried out for codon and structure, for minimum free energy of RNA folding, and the program can screen thousands of sequences (FIG. 9A). mRNA from a fraction of the sequences are now prepared and tested.

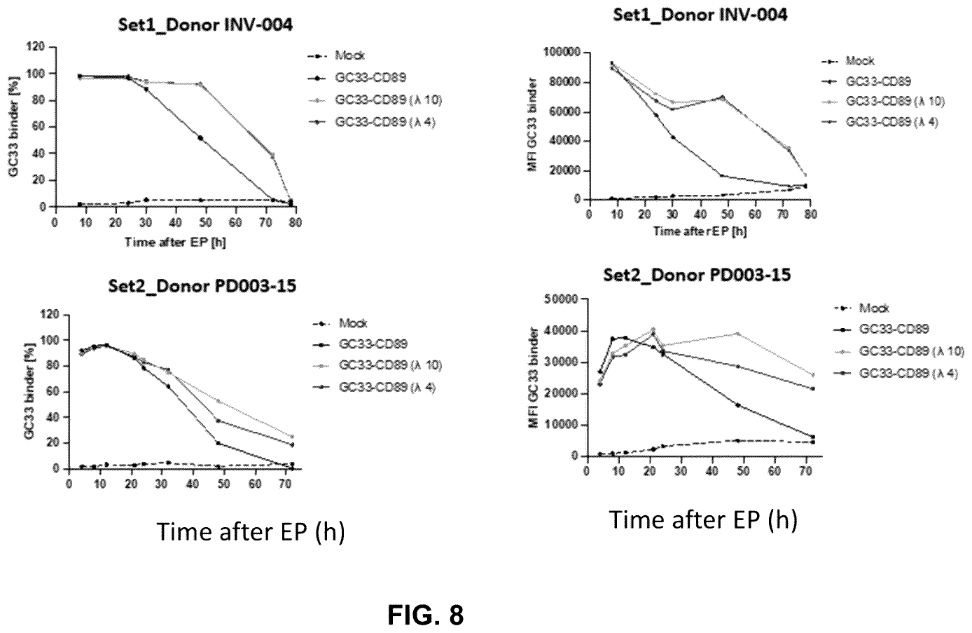

FIGS. 8 and 9B illustrate efforts from these optimizations. Sequences for mRNA design were evaluated in CAI weight scales of lambda 10, 4 and 2. The RNA sequence optimizations include testing and screening coding regions and preparing the mRNA with suitable UTR sequences as tested in the previously described Example for each coding polypeptide. FIG. 9B left bar diagram summarizes an expression pattern of a CFP polypeptide of interest (denoted as XXXX, masked)—with the CD89 transmembrane sequence as disclosed in the specification, and optimized in lambda 10, 4 or 2 parameters. The effect on expression of the polypeptide on myeloid cells is indicated as fold change over the basic non-optimized design (Base construct). FIG. 8 illustrates a study with anti-GPC3 binder CFP sequences, (e.g. sequences provided, SEQ ID NO. 11 or SEQ ID NO. 15), where further optimization using the respective AI based algorithms, the expression of the polypeptide is prolonged. Expression was tested from human donor PBMC cell samples. In FIG. 9B (right top, right bottom) are two additional examples of optimized mRNA encoding CFP polypeptides which additionally comprise miRNA binding sites—showing higher expression in myeloid cells (top right) in preference over NK cells (bottom right), and also prolonged expression (right top graph) over lower graph (NK cells).

An exemplary optimized lambda 10 anti-GPC3 binding
CFP mRNA coding sequence is:
(SEQ ID NO: 25)
ATGTGGCTGCAGTCTCTGCTGCTGCTGGGGACCGTGGCCTGCAGCATCTCCCAGG
TGCAGCTGGTGCAGAGCGGCGCCGAGGTGAAGAAGCCCGGCGCCAGCGTGAAGGTGAG
CTGCAAGGCCAGCGGCTACACCTTCACCGACTACGAGATGCACTGGGTGCGGCAGGCC
CCCGGCCAGGGCCTGGAGTGGATGGGCGCCCTGGACCCCAAGACCGGCGACACCGCCT
ACAGCCAGAAGTTCAAGGGCAGGGTGACCCTGACTGCCGACAAGAGCACCTCCACCGC
CTACATGGAGCTGTCCTCCCTGACCAGCGAGGACACCGCCGTGTACTACTGCACCAGGT
TCTACAGCTACACCTACTGGGGCCAGGGCACCCTGGTGACAGTGAGCTCCGGCGGCGG
CGGGAGCGGCGGGGGCGGCAGCGGGGGCGGGGGCTCTGACGTGGTGATGACCCAGAG
CCCCCTGTCCCTGCCTGTGACCCCCGGCGAGCCCGCCAGCATCAGCTGCCGGAGCTCCC
AGAGCCTGGTGCACAGCAACCGGAACACCTACCTGCACTGGTACCTGCAGAAGCCCGG
CCAGTCCCCCCAGCTGCTGATCTACAAGGTGAGCAATCGGTTCAGCGGCGTGCCAGACC
GGTTCTCCGGCTCTGGCAGCGGCACCGACTTCACCCTGAAGATCAGCAGGGTGGAGGCC
GAGGACGTGGGGGTGTACTACTGCAGTCAGAACACCCACGTGCCCCCCACCTTCGGCCA
GGGCACCAAGCTGGAGATCAAGGGGTCCGGCGGCAGCGACTCCATCCACCAGGACTAC
ACCACCCAGAACCTGATCCGGATGGCCGTGGCCGGCCTGGTGCTGGTGGCCCTGCTGGC
CATCCTGGTGGAGAACTGGCACAGCCACACCGCCCTGAACAAGGAGGCCAGCGCCGAT
GTGGCTGAGCCCTCCTGGAGCCAGCAGATGTGCCAGCCCGGCCTGACCTTCGCCAGGAC
CCCCAGCGTGTGCAAGTGA
An exemplary optimized lambda 4 anti-GPC3 binding
CFP mRNA coding sequence is:
(SEQ ID NO: 26)
ATGTGGCTGCAGTCTCTGCTGCTGCTGGGGACCGTGGCCTGCAGCATCTCCCAGG
TGCAGCTCGTGCAGAGCGGCGCCGAGGTGAAGAAGCCCGGCGCCTCTGTGAAGGTGAG
CTGCAAGGCCAGCGGCTACACCTTCACAGACTACGAGATGCACTGGGTGCGGCAGGCC
CCCGGCCAGGGCCTGGAGTGGATGGGCGCCCTGGACCCCAAGACCGGCGACACCGCCT
ACAGCCAGAAGTTCAAGGGGCGCGTGACTCTGACTGCAGACAAGAGCACCTCCACCGC
CTACATGGAGCTGAGTTCCCTGACCTCAGAGGATACAGCCGTGTACTACTGTACACGGT
TCTACTCCTACACCTACTGGGGTCAGGGAACTCTGGTGACCGTGTCCTCTGGGGGGGGC
GGCAGCGGAGGCGGGGGCTCCGGGGGCGGGGGCAGTGACGTGGTGATGACCCAGTCAC
CACTGTCACTGCCCGTGACCCCCGGAGAACCCGCCTCCATCAGCTGCCGCTCCTCCCAG
AGCCTGGTGCACAGCAACCGGAACACCTACCTGCACTGGTACCTGCAGAAGCCAGGCC
AGTCTCCACAGCTGCTGATCTACAAGGTGAGCAATCGGTTCAGCGGCGTGCCAGACCGG
TTCTCCGGCTCTGGCAGCGGCACCGATTTCACCCTGAAGATCAGCAGGGTGGAGGCTGA
GGACGTGGGGGTGTACTACTGCAGTCAGAACACTCACGTGCCCCCAACTTTTGGCCAGG
GCACCAAGCTGGAGATCAAGGGGTCCGGGGGGTCCGACTCCATCCATCAGGACTACAC
CACCCAGAACCTGATCCGGATGGCCGTGGCCGGCCTGGTGCTGGTGGCCCTGCTGGCCA
TCCTGGTGGAGAACTGGCACAGCCACACAGCGCTGAACAAGGAGGCCAGCGCTGATGT
GGCTGAGCCCTCCTGGAGCCAGCAGATGTGCCAGCCAGGGCTGACCTTCGCCAGGACC
CCCAGCGTGTGCAAGTGA

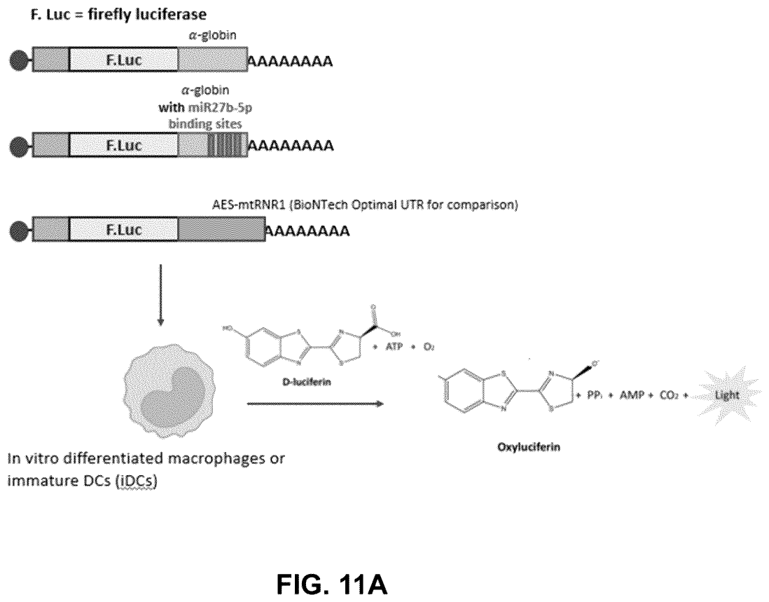

In an effort to improving the protocol further, Applicants used a combination of carefully selected miRNA sites. mRNA expression and longevity were found to improve considerably, and the resultant expression was unprecedented high. Applicants found that using alpha-globin 3′-UTR, and with a codon optimization and insertion of microRNA 27b-5p binding sites site demonstrated that led to very high longevity in primary monocytes. The miRNA site sequences are incorporated in the middle of the globin sequence (FIG. 10). In this example coding sequence has the optimized lambda 10 sequence encoding the anti-GPC3 binding CFP mRNA and has a 3′ UTR sequence with or without the miR-27b, wherein the sequence of the 3′ UTR is the alpha globin 3′UTR, and the sequence with embedded miR sequences is shown in SEQ ID NO: 21.

The extended half-life observed with the alpha-globin 3′ UTR construct with the miR sequences was highly significant, unexpected and surprising.

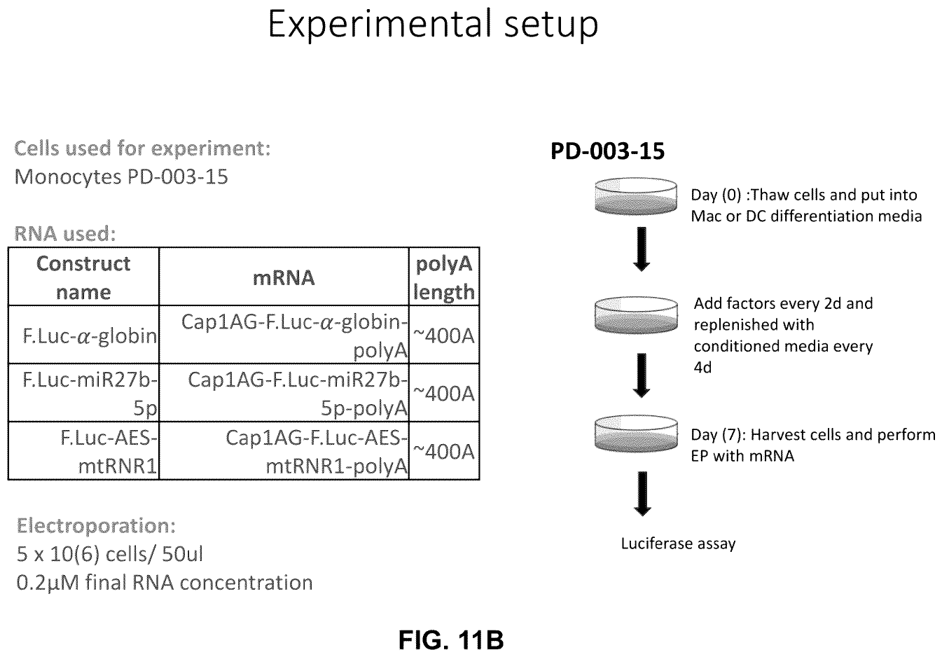

Additional experiments were performed to understand if the 3′ UTR contributes to extended expression of a generic mRNA. For this a sequence encoding firefly luciferase (F. Luc) was incorporated in the recombinant mRNA design replacing the CFP encoding sequence. A generalized view is shown in FIG. 11, with the corresponding experimental set up. (FIGS. 11A and 11B). Alpha-globin 3′UTR+miR27b-5p binding sites x4 CTCGAGTGATAATAGGCTGGAGCCTCGGTGGCCATGCTTCTTGCCCCTTGGGCCTCCCC CCA GCCCCTCCTCCCCTTCCTGCACCCGTACCCCCttcaccaatcagctaagctctCGATttcaccaatcagct aagctctTCTAGAttcaccaatcagctaagctctTCACttcaccaatcagctaagctctGTGGTCTTTGAATAAAGTCT GAGTGGGCGGCGAAGACAATAAGC (SEQ ID NO: 21) (miR sites are in lower case; upper case letters are from αglobin 3′UTR)

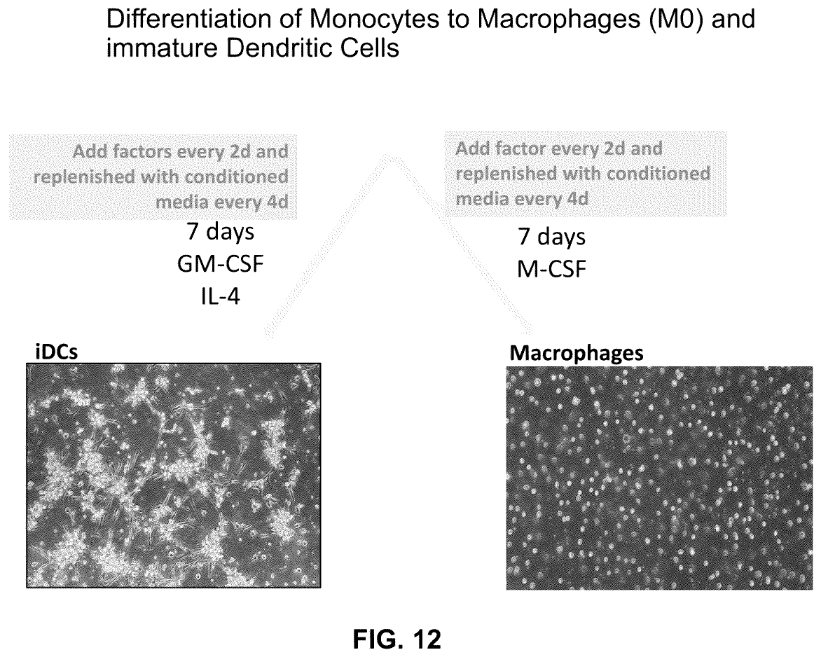

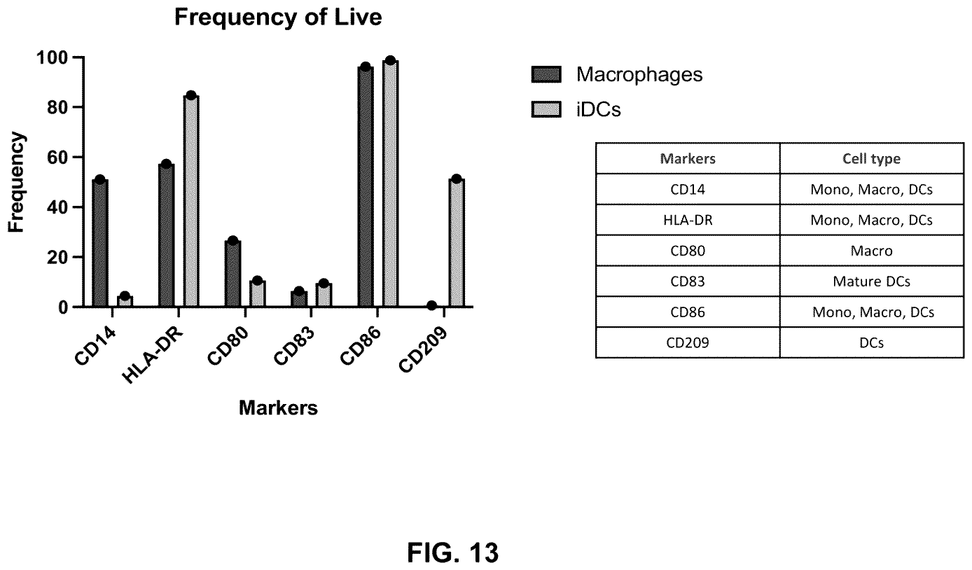

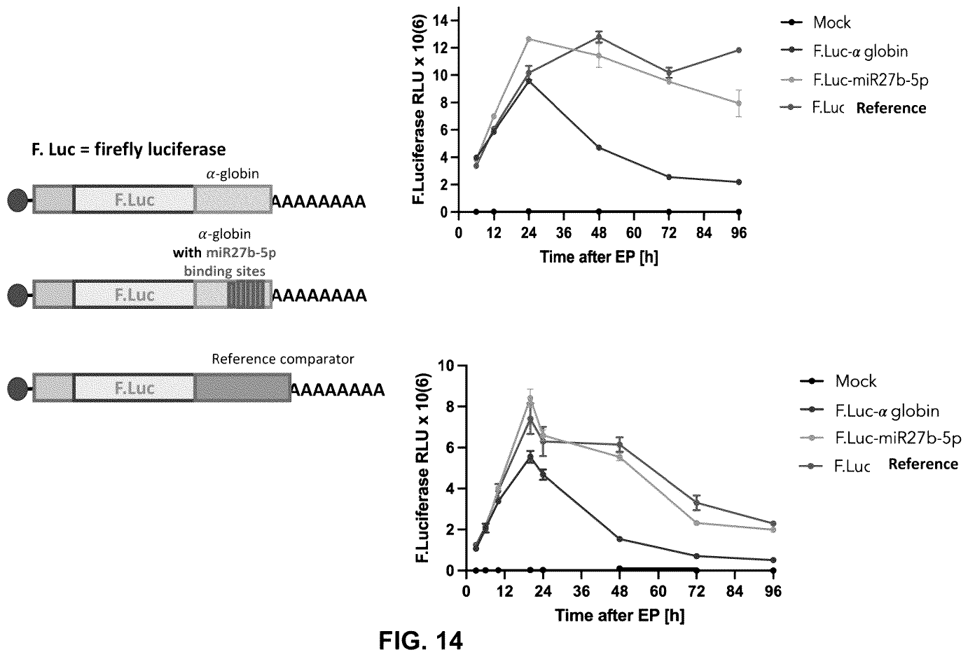

Similar protocol was followed for donor derived monocytes and immature DCs cultured in vitro. Prolonged sustenance of the cells in culture for the myeloid cells and their differentiation was facilitated by addition of the growth factors and cytokines, well known in the art for macrophage and DC culture in vitro. (FIG. 12). The cellular phenotypes of macrophage and iDCs in the culture were identified as shown in FIG. 13. It was shown that the use of the 3′ UTRs with the miR27b-5p binding sites extended luciferase expression greatly. A reference construct was used for comparison, which is a high performing comparator. The performance of the designed and optimized 3′ UTR worked at least as good as the comparator. (FIG. 14). These results show that the designed mRNA containing modified UTRs with the miR target sites not only conferred cell-specific expression of an exogenous polypeptide sequence encoded by a sequence in the mRNA construct, but also conferred increased expression of the exogenous polypeptide over a surprisingly longer period.

1-47. (canceled)

48. A composition for expressing an exogenous polypeptide encoded by an engineered polynucleic acid in a myeloid cell, comprising an engineered polynucleic acid comprising: (a) a sequence encoding the exogenous polypeptide, and (b) a miRNA target sequence targeting miR-192, miR-27b, or miR-10a; wherein the miRNA sequence is operably linked with the sequence encoding the exogenous polypeptide; wherein upon contacting a cell population comprising myeloid cells and non-myeloid cells with the composition, the exogenous polypeptide is expressed in the myeloid cells of the cell population, and the exogenous polypeptide is not substantially expressed in the non-myeloid cells of the cell population.

49. The composition of claim 48, wherein the miRNA target sequence comprises a sequence with at least 90% identity to any one of SEQ ID NOs: 4-9.

50. The composition of claim 48, wherein the miRNA target sequence targets miR-27-5p.

51. The composition of claim 50, wherein the miRNA target sequence comprises a sequence with at least 90% identity to SEQ ID NO: 6.

52. The composition of claim 51, wherein the miRNA target sequence comprises a sequence of UUCACCAAUCAGCUAAGCUCU (nucleotides 2-22 of SEQ ID NO: 6).

53. The composition of claim 48, wherein the engineered polynucleic acid is an mRNA comprising a 5′-UTR and a 3′-UTR, and wherein the miRNA target sequence is incorporated in the 5′-UTR or the 3′-UTR.

54. The composition of claim 53, wherein the miRNA target sequence is incorporated in the 3′UTR.

55. The composition of claim 54, wherein the 3′UTR comprises at least 2 copies of the miRNA target sequence.

56. The composition of claim 55, wherein the 3′UTR comprises at least 4 copies of the miRNA target sequence.

57. The composition of claim 56, wherein the 3′ UTR comprises a sequence that is at least 90% identical to SEQ ID NO: 99.

58. The composition of claim 57, wherein the 3′UTR comprises the sequence according to SEQ ID NO: 99.

59. The composition of claim 53, wherein the 5′ UTR comprises a sequence that is at least 90% identical to SEQ ID NO: 98.

60. The composition of claim 59, wherein the 5′ UTR comprises the sequence according to SEQ ID NO: 98.

61. The composition of claim 48, wherein the exogenous polypeptide is a chimeric fusion protein (CFP), wherein the CFP comprises: (i) an extracellular domain comprising an antigen binding domain that specifically binds to a cancer antigen; (ii) a transmembrane domain; and (iii) an intracellular domain.

62. The composition of claim 61, wherein the cancer antigen is TROP2, GPC3, or HER2.

63. The composition of claim 61, wherein the transmembrane domain of the CFP comprises a sequence that is at least 90% identical to LIRMAVAGLVLVALLAILV (SEQ ID NO: 34).

64. The composition of claim 61, wherein the CFP comprises a sequence that is at least 90% identical to SEQ ID NO: 18.

65. The composition of claim 48, further comprising a nucleic acid delivery vehicle encapsulating the engineered polynucleic acid, and wherein the nucleic acid delivery vehicle is a lipid nanoparticle.

66. A method of expressing an exogenous polypeptide encoded by an engineered polynucleic acid in a myeloid cell, the method comprising: contacting a cell population with the composition of claim 48; wherein the cell population comprises myeloid cells and non-myeloid cells; wherein the exogenous polypeptide is expressed in the myeloid cells of the cell population, and wherein the exogenous polypeptide is not substantially expressed in the non-myeloid cells of the cell population.

67. A pharmaceutical composition, comprising the composition of claim 48 and a pharmaceutically acceptable excipient.

68. A method of treating a disease in a subject in need thereof, the method comprising administering to the subject the pharmaceutical composition of claim 67.


SEQUENCE LISTING (99 sequences)

SEQ ID NO: 1 - DNA, 110 bp, Homo sapiens
  source 1..110: mol_type = genomic DNA
gccgagaccgagtgcacagggctctgacctatgaattgacagccagtgctctcgtctcccctctggctgccaattccataggtcacaggtatgttcgcctcaatgccagc

SEQ ID NO: 2 - DNA, 97 bp, Homo sapiens
  source 1..97: mol_type = genomic DNA
acctctctaacaaggtgcagagcttagctgattggtgaacagtgattggtttccgctttgttcacagtggctaagttctgcacctgaagagaaggtg

SEQ ID NO: 3 - DNA, 110 bp, Homo sapiens
  source 1..110: mol_type = genomic DNA
gatctgtctgtcttctgtatataccctgtagatccgaatttgtgtaaggaattttgtggtcacaaattcgtatctaggggaatatgtagttgacataaacactccgctct

SEQ ID NO: 4 - RNA, 21 nt, Homo sapiens
  source 1..21: mol_type = genomic RNA
ggctgtcaattcataggtcag

SEQ ID NO: 5 - RNA, 22 nt, Homo sapiens
  source 1..22: mol_type = genomic RNA
ctgtgacctatggaattggcag

SEQ ID NO: 6 - RNA, 22 nt, Homo sapiens
  source 1..22: mol_type = genomic RNA
gttcaccaatcagctaagctct

SEQ ID NO: 7 - RNA, 21 nt, Homo sapiens
  source 1..21: mol_type = genomic RNA
gcagaacttagccactgtgaa

SEQ ID NO: 8 - RNA, 23 nt, Homo sapiens
  source 1..23: mol_type = genomic RNA
cacaaattcggatctacagggta

SEQ ID NO: 9 - RNA, 22 nt, Homo sapiens
  source 1..22: mol_type = genomic RNA
tattcccctagatacgaatttg

SEQ ID NO: 10 - DNA, 239 bp, synthetic construct
  source 1..239: mol_type = other DNA
ctcgagtgataataggctggagcctcggtggccatgcttcttgccccttgggcctccccccagcccctcctccccttcctgcacccgtaccccccaaacaccattgtcacactccacgatcaaacaccattgtcacactccatctagacaaacaccattgtcacactccatcaccaaacaccattgtcacactccagtggtctttgaataaagtctgagtgggcggcgaagacaatagc

SEQ ID NO: 11 - protein, 335 aa, synthetic construct
MWLQSLLLLGTVACSISQVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGSGGSDSIHQDYTTQNLIRMAVAGLVLVALLAILVENWHSHTALNKEASADVAEPSWSQQMCQPGLTFARTPSVCK

SEQ ID NO: 12 - protein, 115 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSS

SEQ ID NO: 13 - protein, 112 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 14 - protein, 71 aa, synthetic construct
DSIHQDYTTQNLIRMAVAGLVLVALLAILVENWHSHTALNKEASADVAEPSWSQQMCQPGLTFARTPSVCK

SEQ ID NO: 15 - protein, 335 aa, synthetic construct
MWLQSLLLLGTVACSISQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGSGGSDSIHQDYTTQNLIRMAVAGLVLVALLAILVENWHSHTALNKEASADVAEPSWSQQMCQPGLTFARTPSVCK

SEQ ID NO: 16 - protein, 115 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 17 - protein, 112 aa, synthetic construct; identical to SEQ ID NO: 13

SEQ ID NO: 18 - protein, 71 aa, synthetic construct; identical to SEQ ID NO: 14

SEQ ID NO: 19 - DNA, 235 bp, synthetic construct
  source 1..235: mol_type = other DNA
ctcgagtgataataggctggagcctcggtggccatgcttcttgccccttgggcctccccccagcccctcctccccttcctgcacccgtacccccggctgtcaattcataggtcagcgatggctgtcaattcataggtcagtctagaggctgtcaattcataggtcagtcacggctgtcaattcataggtcaggtggtctttgaataaagtctgagtgggcggcgaagacaatagc

SEQ ID NO: 20 - DNA, 243 bp, synthetic construct
  source 1..243: mol_type = other DNA
ctcgagtgataataggctggagcctcggtggccatgcttcttgccccttgggcctccccccagcccctcctccccttcctgcacccgtacccccctgtgacctatggaattggcaggcgatctgtgacctatggaattggcaggtctagactgtgacctatggaattggcaggtcacctgtgacctatggaattggcagggtggtctttgaataaagtctgagtgggcggcgaagacaatagc

SEQ ID NO: 21 - DNA, 235 bp, synthetic construct
  source 1..235: mol_type = other DNA
ctcgagtgataataggctggagcctcggtggccatgcttcttgccccttgggcctccccccagcccctcctccccttcctgcacccgtacccccttcaccaatcagctaagctctcgatttcaccaatcagctaagctcttctagattcaccaatcagctaagctcttcacttcaccaatcagctaagctctgtggtctttgaataaagtctgagtgggcggcgaagacaatagc

SEQ ID NO: 22 - DNA, 235 bp, synthetic construct
  source 1..235: mol_type = other DNA
ctcgagtgataataggctggagcctcggtggccatgcttcttgccccttgggcctccccccagcccctcctccccttcctgcacccgtacccccgcagaacttagccactgtgaacgatgcagaacttagccactgtgaatctagagcagaacttagccactgtgaatcacgcagaacttagccactgtgaagtggtctttgaataaagtctgagtgggcggcgaagacaatagc

SEQ ID NO: 23 - DNA, 243 bp, synthetic construct
  source 1..243: mol_type = other DNA
ctcgagtgataataggctggagcctcggtggccatgcttcttgccccttgggcctccccccagcccctcctccccttcctgcacccgtaccccccacaaattcggatctacagggtacgatcacaaattcggatctacagggtatctagacacaaattcggatctacagggtatcaccacaaattcggatctacagggtagtggtctttgaataaagtctgagtgggcggcgaagacaatagc

SEQ ID NO: 24 - DNA, 239 bp, synthetic construct
  source 1..239: mol_type = other DNA
ctcgagtgataataggctggagcctcggtggccatgcttcttgccccttgggcctccccccagcccctcctccccttcctgcacccgtaccccctattcccctagatacgaatttgcgattattcccctagatacgaatttgtctagatattcccctagatacgaatttgtcactattcccctagatacgaatttggtggtctttgaataaagtctgagtgggcggcgaagacaatagc

SEQ ID NO: 25 - DNA, 1008 bp, synthetic construct
  source 1..1008: mol_type = other DNA
atgtggctgcagtctctgctgctgctggggaccgtggcctgcagcatctcccaggtgcagctggtgcagagcggcgccgaggtgaagaagcccggcgccagcgtgaaggtgagctgcaaggccagcggctacaccttcaccgactacgagatgcactgggtgcggcaggcccccggccagggcctggagtggatgggcgccctggaccccaagaccggcgacaccgcctacagccagaagttcaagggcagggtgaccctgactgccgacaagagcacctccaccgcctacatggagctgtcctccctgaccagcgaggacaccgccgtgtactactgcaccaggttctacagctacacctactggggccagggcaccctggtgacagtgagctccggcggcggcgggagcggcgggggcggcagcgggggcgggggctctgacgtggtgatgacccagagccccctgtccctgcctgtgacccccggcgagcccgccagcatcagctgccggagctcccagagcctggtgcacagcaaccggaacacctacctgcactggtacctgcagaagcccggccagtccccccagctgctgatctacaaggtgagcaatcggttcagcggcgtgccagaccggttctccggctctggcagcggcaccgacttcaccctgaagatcagcagggtggaggccgaggacgtgggggtgtactactgcagtcagaacacccacgtgccccccaccttcggccagggcaccaagctggagatcaaggggtccggcggcagcgactccatccaccaggactacaccacccagaacctgatccggatggccgtggccggcctggtgctggtggccctgctggccatcctggtggagaactggcacagccacaccgccctgaacaaggaggccagcgccgatgtggctgagccctcctggagccagcagatgtgccagcccggcctgaccttcgccaggacccccagcgtgtgcaagtga

SEQ ID NO: 26 - DNA, 1008 bp, synthetic construct
  source 1..1008: mol_type = other DNA
atgtggctgcagtctctgctgctgctggggaccgtggcctgcagcatctcccaggtgcagctcgtgcagagcggcgccgaggtgaagaagcccggcgcctctgtgaaggtgagctgcaaggccagcggctacaccttcacagactacgagatgcactgggtgcggcaggcccccggccagggcctggagtggatgggcgccctggaccccaagaccggcgacaccgcctacagccagaagttcaaggggcgcgtgactctgactgcagacaagagcacctccaccgcctacatggagctgagttccctgacctcagaggatacagccgtgtactactgtacacggttctactcctacacctactggggtcagggaactctggtgaccgtgtcctctggggggggcggcagcggaggcgggggctccgggggcgggggcagtgacgtggtgatgacccagtcaccactgtcactgcccgtgacccccggagaacccgcctccatcagctgccgctcctcccagagcctggtgcacagcaaccggaacacctacctgcactggtacctgcagaagccaggccagtctccacagctgctgatctacaaggtgagcaatcggttcagcggcgtgccagaccggttctccggctctggcagcggcaccgatttcaccctgaagatcagcagggtggaggctgaggacgtgggggtgtactactgcagtcagaacactcacgtgcccccaacttttggccagggcaccaagctggagatcaaggggtccggggggtccgactccatccatcaggactacaccacccagaacctgatccggatggccgtggccggcctggtgctggtggccctgctggccatcctggtggagaactggcacagccacacagcgctgaacaaggaggccagcgctgatgtggctgagccctcctggagccagcagatgtgccagccagggctgaccttcgccaggacccccagcgtgtgcaagtga

SEQ ID NO: 27 - RNA, 200 nt, synthetic construct
  source 1..200: mol_type = other RNA
  misc_difference 1..200: note = This sequence may encompass 50-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - protein, 37 aa, Homo sapiens
GLAVSTISSFFPPGYQVSFCLVMVLLFAVDTGLYFSV

SEQ ID NO: 29 - protein, 17 aa, Homo sapiens
GLAVSTISSFFPPGYQV

SEQ ID NO: 30 - protein, 20 aa, Homo sapiens
SFCLVMVLLFAVDTGLYFSV

SEQ ID NO: 31 - protein, 25 aa, Homo sapiens
KTNIRSSTRDWKDHKFKWRKDPQDK

SEQ ID NO: 32 - protein, 28 aa, Homo sapiens
IHQDYTTQNLIRMAVAGLVLVALLAILV

SEQ ID NO: 33 - protein, 9 aa, Homo sapiens
IHQDYTTQN

SEQ ID NO: 34 - protein, 19 aa, Homo sapiens
LIRMAVAGLVLVALLAILV

SEQ ID NO: 35 - protein, 41 aa, Homo sapiens
ENWHSHTALNKEASADVAEPSWSQQMCQPGLTFARTPSVCK

SEQ ID NO: 36 - protein, 116 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 37 - protein, 107 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 38 - protein, 240 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 39 - protein, 46 aa, synthetic construct
LYCRRLKIQVRKAAITSYEKSDGVYTGLSTRNQETYETLKHEKPPQ

SEQ ID NO: 40 - protein, 45 aa, synthetic construct
LYCRLKIQVRKAAITSYEKSDGVYTGLSTRNQETYETLKHEKPPQ

SEQ ID NO: 41 - protein, 42 aa, synthetic construct
RLKIQVRKAAITSYEKSDGVYTGLSTRNQETYETLKHEKPPQ

SEQ ID NO: 42 - protein, 35 aa, synthetic construct
YEDMRGILYAAPQLRSIRGQPGPNHEEDADSYENM

SEQ ID NO: 43 - protein, 62 aa, synthetic construct
KKVAKKPTNKAPHPKQEPQEINFPDDLPGSNTAAPVQETLHGCQPVTQEDGKESRISVQERQ

SEQ ID NO: 44 - protein, 21 aa, synthetic construct
IYIWAPLAGTCGVLLLSLVIT

SEQ ID NO: 45 - protein, 24 aa, synthetic construct
IYIWAPLAGTCGVLLLSLVITLYC

SEQ ID NO: 46 - protein, 62 aa, synthetic construct
ALSNSIMYFSHFVPVFLPAKPTTTPAPRPPTPAPTIASQPLSLRPEACRPAAGGAVHTRGLD

SEQ ID NO: 47 - protein, 130 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 48 - protein, 108 aa, synthetic construct
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 49 - protein, 255 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESSGGGGSGGGGSGGGGSLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 50 - protein, 17 aa, synthetic construct
MWLQSLLLLGTVACSIS

SEQ ID NO: 51 - protein, 27 aa, synthetic construct
FWVLVVVGGVLACYSLLVTVAFIIFWV

SEQ ID NO: 52 - protein, 26 aa, synthetic construct
IYLIIGICGGGSLLMVFVALLVFYIT

SEQ ID NO: 53 - protein, 25 aa, synthetic construct
ILLPLIIGLILLGLLALVLIAFCII

SEQ ID NO: 54 - protein, 219 aa, synthetic construct
QRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 55 - protein, 171 aa, synthetic construct
PLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 56 - protein, 211 aa, synthetic construct
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 57 - protein, 86 aa, synthetic construct
ALSNSIMYFSHFVPVFLPAKPTTTPAPRPPTPAPTIASQPLSLRPEACRPAAGGAVHTRGLDIYIWAPLAGTCGVLLLSLVITLYC

SEQ ID NO: 58 - protein, 83 aa, synthetic construct
ALSNSIMYFSHFVPVFLPAKPTTTPAPRPPTPAPTIASQPLSLRPEACRPAAGGAVHTRGLDIYIWAPLAGTCGVLLLSLVIT

SEQ ID NO: 59 - protein, 432 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 60 - protein, 436 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 61 - protein, 454 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQKKVAKKPTNKAPHPKQEPQEINFPDDLPGSNTAAPVQETLHGCQPVTQEDGKESRISVQERQ

SEQ ID NO: 62 - protein, 607 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSMSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 63 - protein, 615 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 64 - protein, 567 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 65 - protein, 17 aa, synthetic construct
SSGGGGSGGGGSGGGGS

SEQ ID NO: 66 - protein, 7 aa, synthetic construct
SGGGGSG

SEQ ID NO: 67 - protein, 4 aa, synthetic construct
SGGG

SEQ ID NO: 68 - protein, 4 aa, synthetic construct
GSGS

SEQ ID NO: 69 - protein, 9 aa, unidentified
SVYDFFVWL

SEQ ID NO: 70 - protein, 25 aa, synthetic construct
GWVKPIIIGHHAYGDQYRATDFVVP

SEQ ID NO: 71 - DNA, 37 bp, synthetic construct
  source 1..37: mol_type = other DNA
gggagacccaagctggctagcgtttaaacttaagctt

SEQ ID NO: 72 - RNA, 37 nt, synthetic construct
  source 1..37: mol_type = other RNA
gggagacccaagctggctagcgtttaaacttaagctt

SEQ ID NO: 73 - DNA, 237 bp, synthetic construct
  source 1..237: mol_type = other DNA
ctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 74 - RNA, 237 nt, synthetic construct
  source 1..237: mol_type = other RNA
ctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 75 - RNA, 47 nt, synthetic construct
  source 1..47: mol_type = other RNA
gggaaataagagagaaaagaagagtaagaagaaatataagagccacc

SEQ ID NO: 76 - RNA, 61 nt, synthetic construct
  source 1..61: mol_type = other RNA
actcctccccatcctctccctctgtccctctgtccctctgaccctgcactgtcccagcacc

SEQ ID NO: 77 - RNA, 50 nt, synthetic construct
  source 1..50: mol_type = other RNA
acatttgcttctgacacaactgtgttcactagcaacctcaaacagacacc

SEQ ID NO: 78 - DNA, 90 bp, synthetic construct
  source 1..90: mol_type = other DNA
cttgcccgccgatatctctgccgggtgactagctgcttcctttctctctcgcgcgcggtgtggtggcagcaggcgcagcccagcctcgaa

SEQ ID NO: 79 - RNA, 90 nt, synthetic construct
  source 1..90: mol_type = other RNA
cttgcccgccgatatctctgccgggtgactagctgcttcctttctctctcgcgcgcggtgtggtggcagcaggcgcagcccagcctcgaa

SEQ ID NO: 80 - DNA, 63 bp, synthetic construct
  source 1..63: mol_type = other DNA
cttcctttttgtccgacatcttgacgaggctgcggtgtctgctgctattctccgagcttcgca

SEQ ID NO: 81 - RNA, 63 nt, synthetic construct
  source 1..63: mol_type = other RNA
cttcctttttgtccgacatcttgacgaggctgcggtgtctgctgctattctccgagcttcgca

SEQ ID NO: 82 - DNA, 58 bp, synthetic construct
  source 1..58: mol_type = other DNA
agcaatcctttctttcagctggagtgctcctcaggagccagccccacccttagaaaag

SEQ ID NO: 83 - RNA, 58 nt, synthetic construct
  source 1..58: mol_type = other RNA
agcaatcctttctttcagctggagtgctcctcaggagccagccccacccttagaaaag

SEQ ID NO: 84 - RNA, 110 nt, synthetic construct
  source 1..110: mol_type = other RNA
tgataataggctggagcctcggtggccatgcttcttgccccttgggcctccccccagcccctcctccccttcctgcacccgtacccccgtggtctttgaataaagtctga

SEQ ID NO: 85 - RNA, 118 nt, synthetic construct
  source 1..118: mol_type = other RNA
caggacacagccttggatcaggacagagacttgggggccatcctgcccctccaacccgacatgtgtacctcagctttttccctcacttgcatcaataaagcttctgtgtttggaacag

SEQ ID NO: 86 - RNA, 114 nt, synthetic construct
  source 1..114: mol_type = other RNA
gctcgctttcttgctgtccaatttctattaaaggttcctttgttccctaagtccaactactaaactgggggatattatgaagggccttgagcatctggattctgcctaataaaa

SEQ ID NO: 87 - RNA, 228 nt, synthetic construct
  source 1..228: mol_type = other RNA
gctcgctttcttgctgtccaatttctattaaaggttcctttgttccctaagtccaactactaaactgggggatattatgaagggccttgagcatctggattctgcctaataaaagctcgctttcttgctgtccaatttctattaaaggttcctttgttccctaagtccaactactaaactgggggatattatgaagggccttgagcatctggattctgcctaataaaa

SEQ ID NO: 88 - DNA, 53 bp, synthetic construct
  source 1..53: mol_type = other DNA
ctggagagaatcacagatgtggaatatttgtcataaataaataatgaaaacct

SEQ ID NO: 89 - DNA, 51 bp, synthetic construct
  source 1..51: mol_type = other DNA
ataggtccaaccagctgtacatttggaaaaataaaactttattaaatcaaa

SEQ ID NO: 90 - DNA, 278 bp, synthetic construct
  source 1..278: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 91 - RNA, 278 nt, synthetic construct
  source 1..278: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 92 - RNA, 243 nt, synthetic construct
  source 1..243: mol_type = other RNA
gctgaagaagtgggaatgggagcactctgtcttctttgctagagaagtggagagaaaatacaaaaggtaaagcagttgagattctctacaacctaaaaaattcctaggtgctattttcttatcctttgtactgtagctaaatgtacctgagacatattagtctttgaaaaataaagttatgtaaggttttttttatctttaaatagctctgtgggttttaacatttttataaagatataccaa

SEQ ID NO: 93 - DNA, 85 bp, Homo sapiens
  source 1..85: mol_type = genomic DNA
ccttagcagagctgtggagtgtgacaatggtgtttgtgtctaaactatcaaacgccattatcacactaaatagctactgctaggc

SEQ ID NO: 94 - DNA, 43 bp, synthetic construct
  source 1..43: mol_type = other DNA
gggagacccaagctggctagcgtttaaacttaagcttgccacc

SEQ ID NO: 95 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 96 - protein, 15 aa, synthetic construct
GGGGSGGGGSGGGGS

SEQ ID NO: 97 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 98 - DNA, 27 bp, synthetic construct
  source 1..27: mol_type = other DNA
aggagacccaagctggctagcgccacc

SEQ ID NO: 99 - DNA, 229 bp, synthetic construct
  source 1..229: mol_type = other DNA
ctcgagtaggctggagcctcggtggccatgcttcttgccccttgggcctccccccagcccctcctccccttcctgcacccgtacccccttcaccaatcagctaagctctcgatttcaccaatcagctaagctcttctagattcaccaatcagctaagctcttcacttcaccaatcagctaagctctgtggtctttgaataaagtctgagtgggcggcgaagacaatagc